(19) United States
(12) Patent Application Publication
(10) Pub. No.: US 2022/0211692 A1
(43) Pub. Date: Jul. 7, 2022

(54) Modulators of the Cystic Fibrosis Transmembrane Conductance Regulator Protein and Methods of Use

(71) Applicant: AbbVie Global Enterprises Ltd., Hamilton (BM)

(72) Inventors: David J. Hardee, Vernon Hills, IL (US); Timothy R. Hodges, Milwaukee, WI (US); Robert G. Schmidt, Antioch, IL (US); Michael R. Schrimpf, Grayslake, IL (US); Xenia B. Searle, Grayslake, IL (US)

(73) Assignee: AbbVie Global Enterprises Ltd., Hamilton (BM)

(21) Appl. No.: 17/554,099

(22) Filed: Dec. 17, 2021

Related U.S. Application Data
(60) Provisional application No. 63/134,398, filed on Jan. 6, 2021.

(51) Int. Cl. A61K 31/4709; C07D 405/12; C07D 405/14; C07D 493/10

(52) U.S. Cl. CPC A61K 31/4709; C07D 493/10; C07D 405/14; C07D 405/12

(57) ABSTRACT

The present invention provides for compounds of Formula (I)

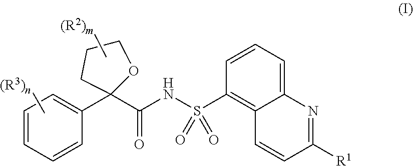

wherein R¹, R², R³, m and n have any of the values defined in the specification, and pharmaceutically acceptable salts thereof, pharmaceutical compositions comprising the same, and methods of treating cystic fibrosis by administering a compound of the invention.

### RELATED APPLICATION

This application claims the benefit of U.S. Provisional Application Ser. No. 63/134,398, filed Jan. 6, 2021. The contents of which are herein incorporated in its entirety.

### FIELD OF THE INVENTION

This invention pertains to substituted tetrahydrofuranyl compounds which are modulators of the Cystic Fibrosis Transmembrane Conductance Regulator (CFTR) protein, useful in treating diseases and conditions mediated and modulated by CFTR. The invention also relates to compositions containing compounds of the invention.

### BACKGROUND

Cystic fibrosis is the most common fatal genetic disease in humans (Bobadilla, J. L., Macek, M., Jr, Fine, J. P., Farrell, P. M., 2002. Cystic fibrosis: a worldwide analysis of CFTR mutations—correlation with incidence data and application to screening. Hum. Mutat. 19, 575-606. doi:10.1002/humu.10041). It is caused by mutations in the gene for CFTR, an anion channel that regulates mucus secretions in epithelial cells of the lungs. In the United States, about one in every 2,500 infants is affected, and up to 10 million individuals carry a single copy of the defective gene without apparent ill effects. In contrast, individuals with two copies of the mutated CFTR gene suffer from the debilitating and fatal effects of CF, including chronic lung infections. Pulmonary exacerbations resulting in hospitalization are common in CF patients. Over time, progressive damage to the lungs from chronic infection can result in a need for lung transplantation. Median age of death in the United States is approximately 31 years (Marshall, B.; Faro, A. et al. Cystic Fibrosis Foundation Patient Registry 2017 Annual Data Report, Cystic Fibrosis Foundation, 2018).

Standard treatment protocols for CF include daily airway clearance regimens, digestive enzyme supplements and the liberal use of antibiotics to control infection. The extensive treatment burden has a substantial effect on quality of life for CF patients and caregivers (Sawicki, G. S.; Sellers, D. E.; Robinson, W. M.; 2009. High Treatment Burden in Adults with Cystic Fibrosis: Challenges to Disease Self-Management. J. Cyst. Fibr. 8, 91-96. https://doi.org/10.1016/j.jcf.2008.09.007). New modulator therapies are available for certain genotypes, including the G55ID and F508del populations, but these are not universally effective and are not approved for many other CFTR mutations. Accordingly, there is a need for novel compounds able to modulate CFTR.

### SUMMARY

In certain embodiments, the invention provides for compounds of Formula (I), or a pharmaceutically acceptable salt thereof,

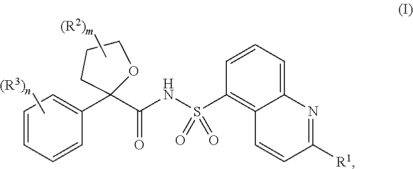

wherein

R¹ is selected from the group consisting of —NH₂, C₁-C₄ alkyl, and C₃-C₇ cycloalkyl;

R² is selected from the group consisting of C₁-C₄ alkyl, —OR^{2a}, and phenyl; wherein the R² phenyl is optionally substituted with one or more R⁴; wherein optionally two R² groups combine to form a C₃-C₆ cycloalkyl or 3-7 membered heterocyclyl;

R^{2a} is selected from the group consisting of C₁-C₄ alkyl, phenyl; wherein the phenyl is optionally substituted with one or more R⁵;

R³ is selected from the group consisting of C₁-C₄ alkyl and —OR^{3a};

R^{3a} is selected from the group consisting of C₁-C₄ alkyl, C₂-C₆ alkoxyalkyl, phenyl, and 5-6 membered heteroaryl; wherein the phenyl and 5-6 membered heteroaryl are optionally substituted with one or more R⁶;

R⁴ is —OR^{4a};

R^{4a} is C₁-C₄ alkyl;

R⁵ is —OR^{5a}.

R^{5a} is C₁-C₄ alkyl;

R⁶ is —OR^{6a};

R^{6a} is selected from the group consisting of C₁-C₄ alkyl and C₁-C₄ haloalkyl;

m is 0, 1, 2, or 3; and

n is 0, 1, or 2.

In certain embodiments of compounds of Formula (I), or a pharmaceutically acceptable salt thereof, R¹ is C₁-C₄ alkyl. In certain embodiments of compounds of Formula (I), or a pharmaceutically acceptable salt thereof, additionally, R^{3a} is C₁-C₄ alkyl; and n is 2. In certain embodiments of compounds of Formula (I), or a pharmaceutically acceptable salt thereof, even further, m is 1 or 2. In certain embodiments of compounds of Formula (I), or a pharmaceutically acceptable salt thereof, still further, R² is selected from the group consisting of C₁-C₄ alkyl and —OR^{2a}. In certain embodiments of compounds of Formula (I), or a pharmaceutically acceptable salt thereof, still further, R² is phenyl optionally substituted with one or more R⁴.

In certain embodiments, the invention provides for compounds of Formula (II), or a pharmaceutically acceptable salt thereof,

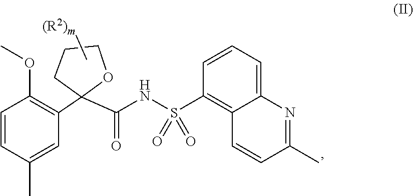

where

R² is selected from the group consisting of C₁-C₄ alkyl, —OR^{2a}, and phenyl; wherein the R² phenyl is optionally substituted with one or more R⁴; wherein optionally two R² groups combine to form a C₃-C₆ cycloalkyl or 3-7 membered heterocyclyl;

R^{2a} is selected from the group consisting of C₁-C₄ alkyl, phenyl; wherein the phenyl is optionally substituted with one or two R⁵;

R⁵ is —OR^{5a}.

R^{5a} is C₁-C₄ alkyl; and

m is 0, 1, 2, or 3.

In certain embodiments of compounds of Formula (II), or a pharmaceutically acceptable salt thereof, m is 1; and R² is phenyl optionally substituted with one or more R⁴.

In certain embodiments, the invention provides for compounds of Formula (III), or a pharmaceutically acceptable salt thereof,

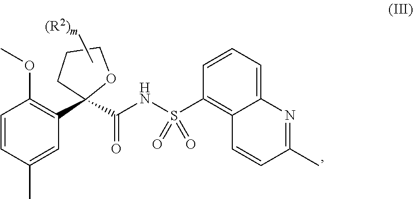

where

R² is selected from the group consisting of C₁-C₄ alkyl, —OR^{2a}, and phenyl; wherein the R² phenyl is optionally substituted with one or more R⁴; wherein optionally two R² groups combine to form a C₃-C₆ cycloalkyl or 3-7 membered heterocyclyl;

R^{2a} is selected from the group consisting of C₁-C₄ alkyl, phenyl; wherein the phenyl is optionally substituted with one or more R⁵;

R⁵ is —OR^{5a}.

R^{5a} is C₁-C₄ alkyl; and

m is 0, 1, 2, or 3.

In certain embodiments of compounds of Formula (III), or a pharmaceutically acceptable salt thereof, m is 1; and R² is phenyl optionally substituted with one or more R⁴.

In certain embodiments, a compound is provided which is

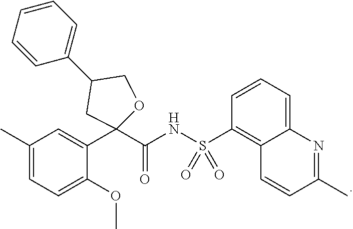

In certain embodiments, a compound, or a pharmaceutically acceptable salt thereof, is provided.

In certain embodiments, a compound is provided which is

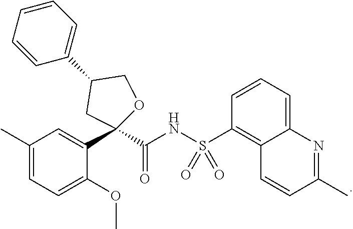

In certain embodiments, a compound, or a pharmaceutically acceptable salt thereof, is provided.

n certain embodiments, a compound is provided which is (2R,4R)-2-(2-methoxy-5-methylphenyl)-N-(2-methylquinoline-5-sulfonyl)-4-phenyloxolane-2-carboxamide. In certain embodiments, the pharmaceutically acceptable salt of (2R,4R)-2-(2-methoxy-5-methylphenyl)-N-(2-methylquinoline-5-sulfonyl)-4-phenyloxolane-2-carboxamide is provided. In certain embodiments, a compound, or a pharmaceutically acceptable salt thereof, is provided.

Certain embodiments of the invention relate to a pharmaceutical composition comprising a therapeutically effective amount of a compound of Formula (I) according to claim 1, or a pharmaceutically acceptable salt thereof, in combination with a pharmaceutically acceptable carrier. Certain embodiments, relate to a pharmaceutical composition comprising a compound of claim 1, or a pharmaceutically acceptable salt thereof, one or more potentiator, and one or more additional correctors.

Certain embodiments of the invention, relate to a method for treating cystic fibrosis in a subject comprising administering a therapeutically effective amount of a compound of claim 1, or a pharmaceutically acceptable salt thereof, to a subject in need thereof. Certain embodiments of the invention, relate to a method for treating cystic fibrosis in a subject comprising administering a therapeutically effective amount of a compound of claim 1, or a pharmaceutically acceptable salt thereof, to a subject in need thereof.

### DETAILED DESCRIPTION

The present invention describes compounds which inhibit the activity of

Disclosed herein are compounds of Formula (I)

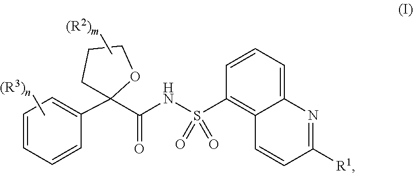

wherein R¹, R², R³, m, and n are defined above in the Summary and below in the Detailed Description. Further, compositions comprising such compounds and methods for treating conditions and disorders using such compounds are also disclosed.

Compounds disclosed herein may contain one or more variable(s) that occur more than one time in any substituent or in the Formulae herein. Definition of a variable on each occurrence is independent of its definition at another occurrence. Further, combinations of substituents are permissible only if such combinations result in stable compounds. Stable compounds are compounds, which can be isolated from a reaction mixture.

### Definitions

Certain terms as used in the specification are intended to refer to the following definitions, as detailed below.

It is noted that, as used in this specification and the intended claims, the singular form “a,” “an,” and “the” include plural referents unless the context clearly dictates otherwise. Thus, for example, reference to “a compound” includes a single compound as well as one or more of the same or different compounds. Reference to “a pharmaceutically acceptable carrier” means a single pharmaceutically acceptable carrier as well as one or more pharmaceutically acceptable carriers, and the like.

As used in the specification and the appended claims, unless specified to the contrary, the following terms have the meaning indicated:

The term “alkoxy,” as used herein, refers to an alkyl group, as defined herein, appended to the parent molecular moiety through an oxygen atom. The alkoxy group may have one, two, three, four, or five carbons unless otherwise specified. Representative examples of alkoxy include, but are not limited to, methoxy, ethoxy, propoxy, 2-propoxy, butoxy, tert-butoxy, and pentyloxy, and the like.

The term “alkoxyalkyl,” as used herein, refers to an alkoxy group, as defined herein, appended to the parent molecular moiety through an alkyl group, as defined herein. The alkoxyalkoxy group may have two, three, four, five, or six carbons unless otherwise specified Representative examples of alkoxyalkyl include, but are not limited to, tert-butoxymethyl, 2-ethoxyethyl, 2-methoxyethyl, and methoxymethyl, and the like.

The term “alkyl,” as used herein, refers to a saturated, straight or branched hydrocarbon chain radical having one, two, three, or four carbons unless otherwise specified. Representative examples of alkyl include, but are not limited to, methyl, ethyl, n-propyl, isopropyl, n-butyl, sec-butyl, isobutyl, tert-butyl, and the like.

The term “cycloalkyl,” as used herein, refers to a saturated hydrocarbon ring radical containing carbon ring atoms. The cycloalkyl is a monocyclic cycloalkyl. The monocyclic cycloalkyl is a carbocyclic ring system containing three, four, five or six carbon atoms, zero heteroatoms and zero double bonds. Examples of monocyclic ring systems include cyclopropyl, cyclobutyl, cyclopentyl, and cyclohexyl.

The term “halo” or “halogen,” as used herein, means Cl, Br, I, and F.

The term “haloalkyl,” as used herein, refers to an alkyl group, as defined herein, in which one or more hydrogen atoms are replaced by halogen having one, two, three, or four carbons unless otherwise specified Representative examples of haloalkyl include, but are not limited to, chloromethyl, 2-fluoroethyl, 2,2-difluoroethyl, fluoromethyl, 2,2,2-trifluoroethyl, trifluoromethyl, difluoromethyl, pentafluoroethyl, trifluorobutyl, trifluoropropyl, and the like.

The term “heteroaryl,” as used herein, refers to an aromatic ring radical containing one or more heteroatoms or a ring system. The monocyclic heteroaryl is a five- or six-membered ring. The five-membered ring contains two double bonds and one or more heteroatoms selected from O, S, and N. The six-membered ring contains three double bonds and one, two, three or four nitrogen atoms. Representative examples of monocyclic heteroaryl include, but are not limited to, furanyl, imidazolyl, isoxazolyl, isothiazolyl, oxadiazolyl, 1,3-oxazolyl, pyridinyl, pyridazinyl, pyrimidinyl, pyrazinyl, pyrazolyl, pyrrolyl, tetrazolyl, thiadiazolyl, 1,3-thiazolyl, thienyl, triazolyl, and triazinyl, and the like.

The term “heteroatom,” as used herein, means a nitrogen, oxygen, or sulfur atom.

The term “heterocycle” or “heterocyclyl” as used herein, refers to a hydrocarbon ring radical wherein at least one carbon atom is replaced by a heteroatom independently selected from the group consisting of O, N, and S. The heterocyclyl ring may be a single ring (monocyclic) or have two or more rings (bicyclic or polycyclic). Monocyclic ring systems are exemplified by any 3- or 4-membered ring containing a heteroatom independently selected from oxygen, nitrogen and sulfur; or a 5-, 6- or 7-membered ring containing one, two or three heteroatoms wherein the heteroatoms are independently selected from nitrogen, oxygen and sulfur. The 5-membered ring has from 0-2 double bonds and the 6- and 7-membered rings have from 0-3 double bonds. Representative examples of heterocyclyl monocyclic ring systems include, but are not limited to, azetidinyl, azepinyl, aziridinyl, diazepinyl, 1,3-dioxolanyl, dioxanyl, dithianyl, furanyl (furyl), imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridinyl, pyrimidinyl, pyridazinyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, thiopyranyl, triazinyl, triazolyl, trithianyl, and the like.

In some instances, the number of carbon atoms in a moiety is indicated by the prefix “Cx-Cy”, wherein x is the minimum and y is the maximum number of carbon atoms in the substituent. Thus, for example, “C1-C6 alkyl” means an alkyl substituent containing from 1 to 6 carbon atoms and “C1-C3 alkyl” means an alkyl substituent containing from 1 to 3 carbon atoms.

In some instances, the number of ring atoms in a moiety is indicated by the prefix “x-y membered”, wherein x is the minimum and y is the maximum number of ring atoms in the substituent. Thus, for example, the term “5- to 6-membered heteroaryl” means a heteroaryl containing 5 to 6 ring atoms.

If a moiety is described as being “optionally substituted,” the moiety may be either (1) not substituted or (2) substituted. If a moiety is described as being optionally substituted with up to a particular number of non-hydrogen radicals, that moiety may be either (1) not substituted; or (2) substituted by up to that particular number of non-hydrogen radicals or by up to the maximum number of substitutable positions on the moiety, whichever is less. Thus, for example, if a moiety is described as a heteroaryl optionally substituted with up to 3 non-hydrogen radicals, then any heteroaryl with less than 3 substitutable positions would be optionally substituted by up to only as many non-hydrogen radicals as the heteroaryl has substitutable positions. To illustrate, tetrazolyl (which has only one substitutable position) would be optionally substituted with up to one non-hydrogen radical. To illustrate further, if an amino nitrogen is described as being optionally substituted with up to 2 non-hydrogen radicals, then a primary amino nitrogen will be optionally substituted with up to 2 non-hydrogen radicals, whereas a secondary amino nitrogen will be optionally substituted with up to only 1 non-hydrogen radical.

With reference to the use of the words “comprise” or “comprises” or “comprising” in this patent application (including the claims), Applicants note that unless the context requires otherwise, those words are used on the basis and clear understanding that they are to be interpreted inclusively, rather than exclusively, and that Applicants intend each of those words to be so interpreted in construing this patent application, including the claims below.

The phrase “pharmaceutical composition” refers to a composition suitable for administration in medical or veterinary use.

The phrase “pharmaceutically acceptable salt” refers to those salts which are, within the scope of sound medical judgement, suitable for use in contact with the tissues of humans and lower animals without undue toxicity, irritation, allergic response and the like and are commensurate with a reasonable benefit/risk ratio.

The terms “prevent,” “preventing,” and “prevention” refer to a method of preventing the onset of a disease and/or its attendant symptoms or barring a subject from acquiring a disease. As used herein, “prevent,” “preventing,” and “prevention” also include delaying the onset of a disease and/or its attendant symptoms and reducing a subject's risk of acquiring or developing a disease or disorder.

The term “stable” refers to compounds that possess stability sufficient to allow manufacture and that maintain the integrity of the compound for a sufficient period of time to be useful for the purpose detailed herein.

If a moiety is described as “substituted,” a non-hydrogen radical is in the place of hydrogen radical of any substitutable atom of the moiety. Thus, for example, a substituted heterocycle moiety is a heterocycle moiety in which at least one non-hydrogen radical is in the place of a hydrogen radical on the heterocycle. It should be recognized that if there are more than one substitution on a moiety, each non-hydrogen radical may be identical or different (unless otherwise stated).

The phrase “therapeutically effective amount” refers to an amount of a compound, or a pharmaceutically acceptable salt thereof, sufficient to prevent the development of or to alleviate to some extent one or more of the symptoms of the condition or disorder being treated when administered for treatment in a particular subject or subject population. The “therapeutically effective amount” may vary depending on the compound, the disease and its severity, and the age, weight, health, etc., of the subject to be treated. For example in a human or other mammal, a therapeutically effective amount may be determined experimentally in a laboratory or clinical setting, or may be the amount required by the guidelines of the United States Food and Drug Administration, or equivalent foreign agency, for the particular disease and subject being treated.

The terms “treat,” “treating,” and “treatment,” as used herein, refer to a method of alleviating or abrogating a disease and/or its attendant symptoms.

The term “one or more” refers to one to five. In certain embodiments, it refers to one or four. In certain embodiments, it refers to one to four. In certain embodiments, it refers to one or three. In certain embodiments, it refers to one to three. In certain embodiments, it refers to one to two. In certain embodiments, it refers to two. In yet other further embodiment, it refers to one.

### Compounds

Compounds of the invention have the general Formula (I) as described above.

In some embodiments, the invention provides compounds of Formula (I), or a pharmaceutically acceptable salt thereof,

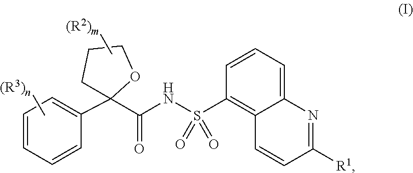

wherein

R¹ is selected from the group consisting of —NH₂, C₁-C₄ alkyl, and C₃-C₇ cycloalkyl;

R² is selected from the group consisting of C₁-C₄ alkyl, —OR^{2a}, and phenyl; wherein the R² phenyl is optionally substituted with one or more R⁴; wherein optionally two R² groups combine to form a C₃-C₆ cycloalkyl or 3-7 membered heterocyclyl;

R^{2a} is selected from the group consisting of C₁-C₄ alkyl, phenyl; wherein the phenyl is optionally substituted with one or more R⁵;

R³ is selected from the group consisting of C₁-C₄ alkyl and —OR^{3a};

R^{3a} is selected from the group consisting of C₁-C₄ alkyl, C₂-C₆ alkoxyalkyl, phenyl, and 5-6 membered heteroaryl; wherein the phenyl and 5-6 membered heteroaryl are optionally substituted with one or more R⁶;

R⁴ is —OR^{4a};

R^{4a} is C₁-C₄ alkyl;

R⁵ is —OR^{5a}.

R^{5a} is C₁-C₄ alkyl;

R⁶ is —OR^{6a};

R^{6a} is selected from the group consisting of C₁-C₄ alkyl and C₁-C₄ haloalkyl;

m is 0, 1, 2, or 3; and

n is 0, 1, or 2.

In certain embodiments of formula (I), or a pharmaceutically acceptable salt thereof, R¹ is C₁-C₄ alkyl; and the remaining variables are as defined for formula (I).

In certain embodiments of formula (I), or a pharmaceutically acceptable salt thereof, R¹ is C₁-C₄ alkyl; R^{3a} is C₁-C₄ alkyl; and n is 2; and the remaining variables are as defined for formula (I).

In certain embodiments of formula (I), or a pharmaceutically acceptable salt thereof, R¹ is C₁-C₄ alkyl; R^{3a} is C₁-C₄ alkyl; m is 1 or 2; and n is 2; and the remaining variables are as defined for formula (I).

In certain embodiments of formula (I), or a pharmaceutically acceptable salt thereof, R¹ is C₁-C₄ alkyl; R² is selected from the group consisting of C₁-C₄ alkyl and —OR^{2a}; R^{3a} is C₁-C₄ alkyl; m is 1 or 2; and n is 2; and the remaining variables are as defined for formula (I).

In certain embodiments of formula (I), or a pharmaceutically acceptable salt thereof, R¹ is C₁-C₄ alkyl; R² is phenyl optionally substituted with one or more R⁴; R^{3a} is C₁-C₄ alkyl; m is 1 or 2; and n is 2; and the remaining variables are as defined for formula (I).

In certain embodiments, the invention provides compounds of Formula (II), or a pharmaceutically acceptable salt thereof,

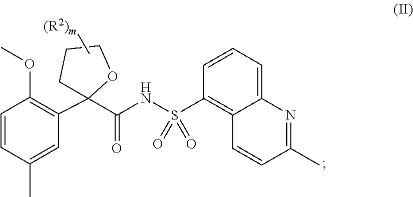

where

R² is selected from the group consisting of C₁-C₄ alkyl, —OR^{2a}, and phenyl; wherein the R² phenyl is optionally substituted with one or more R⁴; wherein optionally two R² groups combine to form a C₃-C₆ cycloalkyl or 3-7 membered heterocyclyl;

R^{2a} is selected from the group consisting of C₁-C₄ alkyl, phenyl; wherein the phenyl is optionally substituted with one or more R⁵;

R⁵ is —OR^{5a}.

R^{5a} is C₁-C₄ alkyl; and

m is 0, 1, 2, or 3.

In certain embodiments of formula (II), or a pharmaceutically acceptable salt thereof, m is 1 or 2; and the remaining variables are as defined for formula (II).

In certain embodiments of formula (II), or a pharmaceutically acceptable salt thereof, R² is phenyl optionally substituted with one or more R⁴; m is 1; and the remaining variables are as defined for formula (II).

In certain embodiments, the invention provides compounds of Formula (III), or a pharmaceutically acceptable salt thereof,

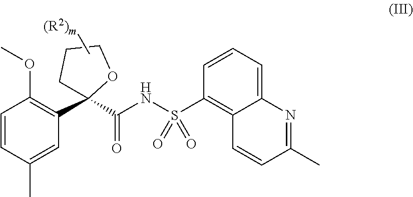

where

R² is selected from the group consisting of C₁-C₄ alkyl, —OR^{2a}, and phenyl; wherein the R² phenyl is optionally substituted with one or more R⁴; wherein optionally two R² groups combine to form a C₃-C₆ cycloalkyl or 3-7 membered heterocyclyl;

R^{2a} is selected from the group consisting of C₁-C₄ alkyl, phenyl; wherein the phenyl is optionally substituted with one or more R⁵;

R⁵ is —OR^{5a}.

R^{5a} is C₁-C₄ alkyl; and

m is 0, 1, 2, or 3.

In certain embodiments of formula (III), or a pharmaceutically acceptable salt thereof, m is 1 or 2; and
the remaining variables are as defined for formula (III).

In certain embodiments of formula (III), or a pharmaceutically acceptable salt thereof, R² is phenyl optionally substituted with one or more R⁴; m is 1; and the remaining variables are as defined for formula (III).

In certain embodiments of formula (I), or a pharmaceutically acceptable salt thereof, the compound is

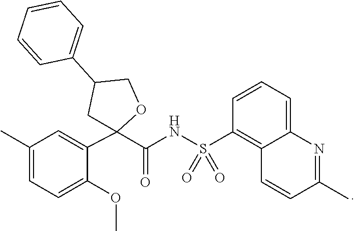

In certain embodiments of formula (I), the compound is

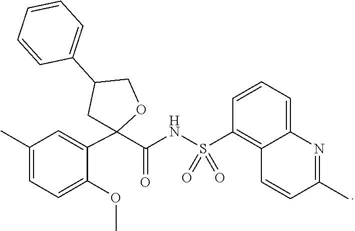

In certain embodiments of formula (I), or a pharmaceutically acceptable salt thereof, the compound is

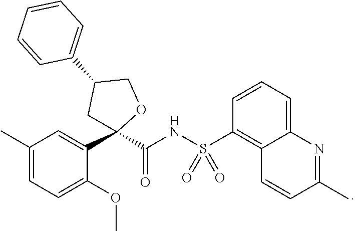

In certain embodiments of formula (I), the compound is

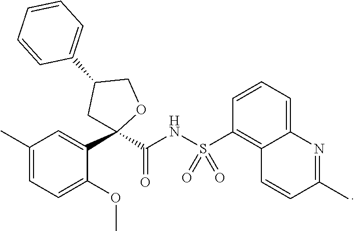

Compound names are assigned by using Name 2019 by Advanced Chemical Development (ACD)/ChemSketch 2019.1.1 naming algorithm, or for some intermediates, using Struct=Name naming algorithm as part of CIEMIDRAW® Professional v. 15.0.0.106.

In certain embodiments a compound, or a pharmaceutically acceptable salt thereof, is provided selected from the group consisting of
(2S)-2-(2-ethoxy-5-methylphenyl)-N-(2-methylquinoline-5-sulfonyl)oxolane-2-carboxamide;
(2R)-2-(2-ethoxy-5-methylphenyl)-N-(2-methylquinoline-5-sulfonyl)oxolane-2-carboxamide;
(2S)-2-(2-methoxy-5-methylphenyl)-4,4-dimethyl-N-(2-methylquinoline-5-sulfonyl)oxolane-2-carboxamide;
7-(2-methoxy-5-methylphenyl)-N-(2-methylquinoline-5-sulfonyl)-2,6-dioxaspiro[3.4]octane-7-carboxamide;
(2R)-2-(2-methoxy-5-methylphenyl)-4,4-dimethyl-N-(2-methylquinoline-5-sulfonyl)oxolane-2-carboxamide;
(2R)—N-(2-cyclopropylquinoline-5-sulfonyl)-2-(2-ethoxy-5-methylphenyl)oxolane-2-carboxamide;
(2S,5S)-2-(2-methoxy-5-methylphenyl)-N-(2-methylquinoline-5-sulfonyl)-5-phenyloxolane-2-carboxamide;
(2R,5S)-2-(2-methoxy-5-methylphenyl)-N-(2-methylquinoline-5-sulfonyl)-5-phenyloxolane-2-carboxamide;
(2R)-2-(2-ethoxy-5-ethylphenyl)-N-(2-methylquinoline-5-sulfonyl)oxolane-2-carboxamide;
(2S)-2-(2-ethoxy-5-ethylphenyl)-N-(2-methylquinoline-5-sulfonyl)oxolane-2-carboxamide;
(2S,5R)-2-(2-methoxy-5-methylphenyl)-N-(2-methylquinoline-5-sulfonyl)-5-phenyloxolane-2-carboxamide;
(2R,5R)-2-(2-methoxy-5-methylphenyl)-N-(2-methylquinoline-5-sulfonyl)-5-phenyloxolane-2-carboxamide;
(2S)-2-{5-ethyl-2-[(2S)-2-methoxypropoxy]phenyl}-N-(2-methylquinoline-5-sulfonyl)oxolane-2-carboxamide;
(2R)-2-{5-ethyl-2-[(2S)-2-methoxypropoxy]phenyl}-N-(2-methylquinoline-5-sulfonyl)oxolane-2-carboxamide;
(6R)-6-(2-methoxy-5-methylphenyl)-N-(2-methylquinoline-5-sulfonyl)-5-oxaspiro[2.4]heptane-6-carboxamide;
(6S)-6-(2-methoxy-5-methylphenyl)-N-(2-methylquinoline-5-sulfonyl)-5-oxaspiro[2.4]heptane-6-carboxamide;
2-(5-ethyl-2-phenoxyphenyl)-N-(2-methylquinoline-5-sulfonyl)oxolane-2-carboxamide;
2-{5-ethyl-2-[(2-methoxypyridin-3-yl)oxy]phenyl}-N-(2-methylquinoline-5-sulfonyl)oxolane-2-carboxamide;
(2R,4R)-2-(2-methoxy-5-methylphenyl)-4-methyl-N-(2-methylquinoline-5-sulfonyl)oxolane-2-carboxamide;
(2R,4S)-2-(2-methoxy-5-methylphenyl)-4-methyl-N-(2-methylquinoline-5-sulfonyl)oxolane-2-carboxamide;
2-(2-{[2-(difluoromethoxy)pyridin-3-yl]oxy}-5-ethylphenyl)-N-(2-methylquinoline-5-sulfonyl)oxolane-2-carboxamide;
(2S)-2-(5-ethyl-2-phenoxyphenyl)-N-(2-methylquinoline-5-sulfonyl)oxolane-2-carboxamide;
(2R)-2-(5-ethyl-2-phenoxyphenyl)-N-(2-methylquinoline-5-sulfonyl)oxolane-2-carboxamide;
(2S)-2-{5-ethyl-2-[(2-methoxypyridin-3-yl)oxy]phenyl}-N-(2-methylquinoline-5-sulfonyl)oxolane-2-carboxamide;
(2R)-2-{5-ethyl-2-[(2-methoxypyridin-3-yl)oxy]phenyl}-N-(2-methylquinoline-5-sulfonyl)oxolane-2-carboxamide;
(2S)-2-(2-{[2-(difluoromethoxy)pyridin-3-yl]oxy}-5-ethylphenyl)-N-(2-methylquinoline-5-sulfonyl)oxolane-2-carboxamide;
(2R)-2-(2-{[2-(difluoromethoxy)pyridin-3-yl]oxy}-5-ethylphenyl)-N-(2-methylquinoline-5-sulfonyl)oxolane-2-carboxamide;
(2R,4S)-4-methoxy-2-(2-methoxy-5-methylphenyl)-N-(2-methylquinoline-5-sulfonyl)oxolane-2-carboxamide;
(2R,4R)-4-methoxy-2-(2-methoxy-5-methylphenyl)-N-(2-methylquinoline-5-sulfonyl)oxolane-2-carboxamide;
(2R,4S)-4-methoxy-2-(2-methoxy-5-methylphenyl)-4-methyl-N-(2-methylquinoline-5-sulfonyl)oxolane-2-carboxamide;
(2S,4R)-4-methoxy-2-(2-methoxy-5-methylphenyl)-4-methyl-N-(2-methylquinoline-5-sulfonyl)oxolane-2-carboxamide;
(2R,4S)-2-(2-methoxy-5-methylphenyl)-N-(2-methylquinoline-5-sulfonyl)-4-phenyloxolane-2-carboxamide;
(2R,4R)-2-(2-methoxy-5-methylphenyl)-N-(2-methylquinoline-5-sulfonyl)-4-phenyloxolane-2-carboxamide;
(2S)—N-(2-aminoquinoline-5-sulfonyl)-2-(2-methoxy-5-methylphenyl)-4,4-dimethyloxolane-2-carboxamide;
(2R)—N-(2-aminoquinoline-5-sulfonyl)-2-(2-methoxy-5-methylphenyl)-4,4-dimethyloxolane-2-carboxamide;
(2S,4S)-2-(2-methoxy-5-methylphenyl)-N-(2-methylquinoline-5-sulfonyl)-4-phenyloxolane-2-carboxamide;
(2S,4R)-2-(2-methoxy-5-methylphenyl)-N-(2-methylquinoline-5-sulfonyl)-4-phenyloxolane-2-carboxamide;
(2R,4S)-2-(2-methoxy-5-methylphenyl)-4-(2-methoxyphenyl)-N-(2-methylquinoline-5-sulfonyl)oxolane-2-carboxamide;
(2R,4R)-2-(2-methoxy-5-methylphenyl)-4-(2-methoxyphenyl)-N-(2-methylquinoline-5-sulfonyl)oxolane-2-carboxamide;
(2R,4R)—N-(2-aminoquinoline-5-sulfonyl)-2-(2-methoxy-5-methylphenyl)-4-(2-methoxyphenyl)oxolane-2-carboxamide;
(2R,4R)—N-(2-aminoquinoline-5-sulfonyl)-2-(2-methoxy-5-methylphenyl)-4-phenyloxolane-2-carboxamide;
(2R,4R)-2-(2-methoxy-5-methylphenyl)-4-(3-methoxyphenyl)-N-(2-methylquinoline-5-sulfonyl)oxolane-2-carboxamide;
(2R,4S)-2-(2-methoxy-5-methylphenyl)-4-(3-methoxyphenyl)-N-(2-methylquinoline-5-sulfonyl)oxolane-2-carboxamide;
(2R,4R)-2-(2-methoxy-5-methylphenyl)-4-(4-methoxyphenyl)-N-(2-methylquinoline-5-sulfonyl)oxolane-2-carboxamide; and
(2R,4S)-2-(2-methoxy-5-methylphenyl)-4-(4-methoxyphenyl)-N-(2-methylquinoline-5-sulfonyl)oxolane-2-carboxamide.

In certain embodiments the compound, or a pharmaceutically acceptable salt thereof, is provided which is (2R,4R)-2-(2-methoxy-5-methylphenyl)-N-(2-methylquinoline-5-sulfonyl)-4-phenyloxolane-2-carboxamide.

In certain embodiments the compound is provided which is (2R,4R)-2-(2-methoxy-5-methylphenyl)-N-(2-methylquinoline-5-sulfonyl)-4-phenyloxolane-2-carboxamide.

Exemplary compounds of Formula (I) include, but are not limited to, the compounds shown in Table 1 below, and pharmaceutically acceptable salts thereof. It is to be understood that when there is a discrepancy between the name of the compound found herein and the structure found in Table 1, the structure in Table 1 shall prevail.

TABLE 1
Exemplary Compounds

| Ex | Structure |
|---|---|
| 1 | 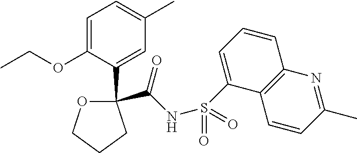 [SMILES: CCOc1ccc(C)cc1[C@@]1(C(=O)NS(=O)(=O)c2cccc3nc(C)ccc23)CCCO1] |
| 2 | 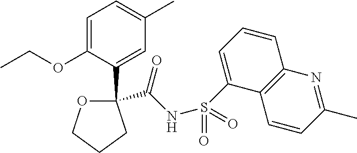 [SMILES: CCOc1ccc(C)cc1[C@@]1(C(=O)NS(=O)(=O)c2cccc3nc(C)ccc23)CCCO1] |
| 3 | 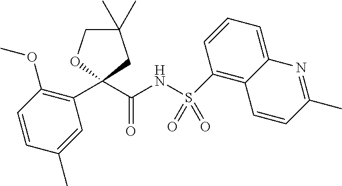 |
| 4 | 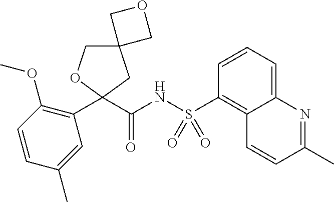 |
| 5 | 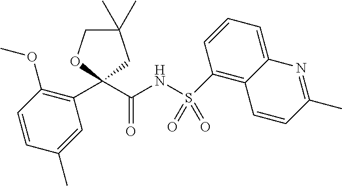 |
| 6 | 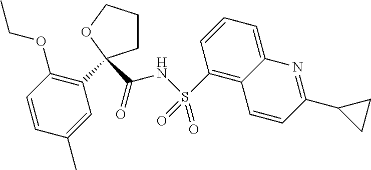 [SMILES: CCOc1ccc(C)cc1[C@@]1(C(=O)NS(=O)(=O)c2cccc3nc(C4CC4)ccc23)CCCO1] |
| 7 | 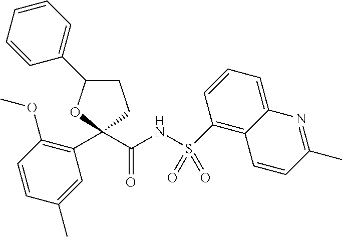 |
| 8 | 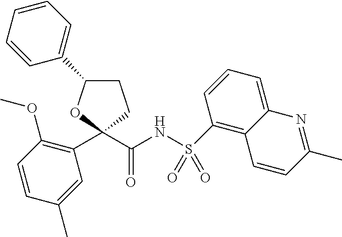 |
| 9 | 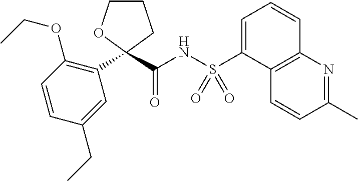 [SMILES: CCOc1ccc(CC)cc1[C@@]1(C(=O)NS(=O)(=O)c2cccc3nc(C)ccc23)CCCO1] |
| 10 | 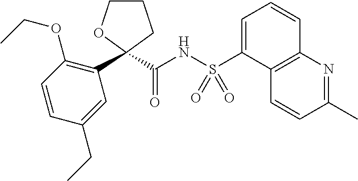 [SMILES: CCOc1ccc(CC)cc1[C@]1(C(=O)NS(=O)(=O)c2cccc3nc(C)ccc23)CCCO1] |
| 11 | 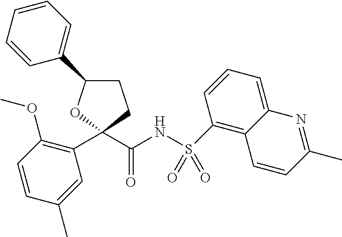 |
| 12 | 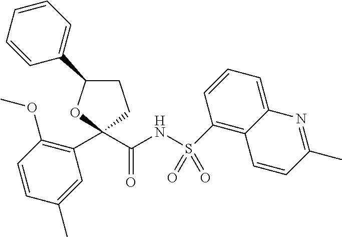 |
| 13 | 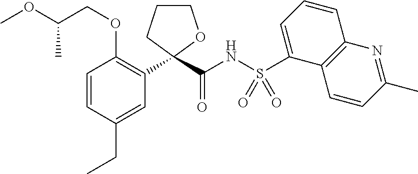 |
| 14 | 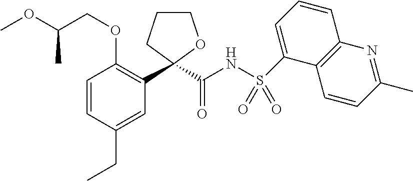 |
| 15 | 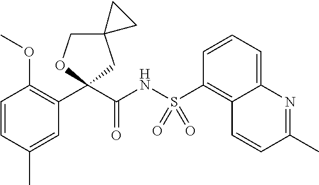 |
| 16 | 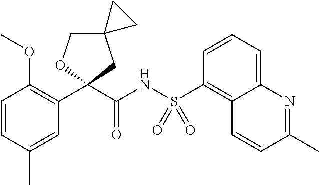 |
| 17 | 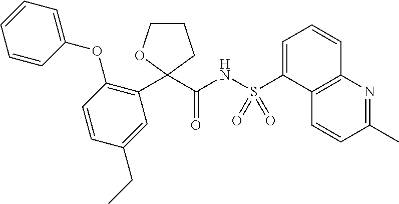 [SMILES: CCc1ccc(Oc2ccccc2)c(C2(C(=O)NS(=O)(=O)c3cccc4nc(C)ccc34)CCCO2)c1] |
| 18 | 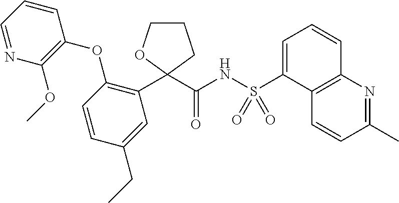 [SMILES: CCc1ccc(Oc2cccnc2OC)c(C2(C(=O)NS(=O)(=O)c3cccc4nc(C)ccc34)CCCO2)c1] |
| 19 | 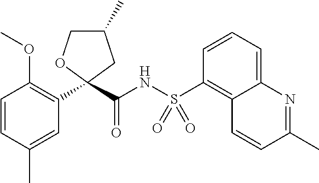 |
| 20 | 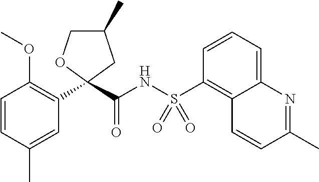 |
| 21 | 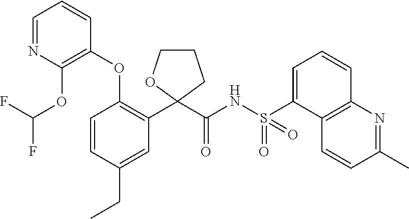 [SMILES: CCc1ccc(Oc2cccnc2OC(F)F)c(C2(C(=O)NS(=O)(=O)c3cccc4nc(C)ccc34)CCCO2)c1] |
| 22 | 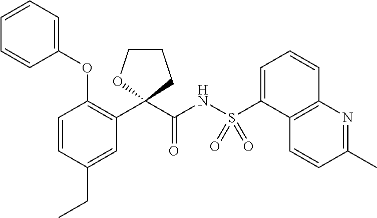 [SMILES: CCc1ccc(Oc2ccccc2)c([C@]2(C(=O)NS(=O)(=O)c3cccc4nc(C)ccc34)CCCO2)c1] |
| 23 | 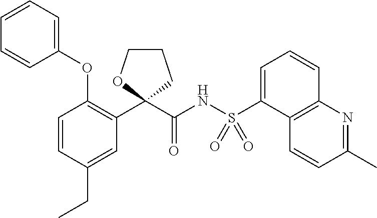 [SMILES: CCc1ccc(Oc2ccccc2)c([C@@]2(C(=O)NS(=O)(=O)c3cccc4nc(C)ccc34)CCCO2)c1] |
| 24 | 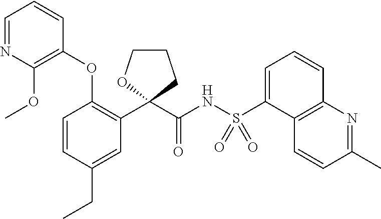 [SMILES: CCc1ccc(Oc2cccnc2OC)c([C@]2(C(=O)NS(=O)(=O)c3cccc4nc(C)ccc34)CCCO2)c1] |
| 25 | 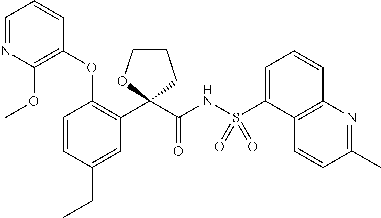 [SMILES: CCc1ccc(Oc2cccnc2OC)c([C@@]2(C(=O)NS(=O)(=O)c3cccc4nc(C)ccc34)CCCO2)c1] |
| 26 | 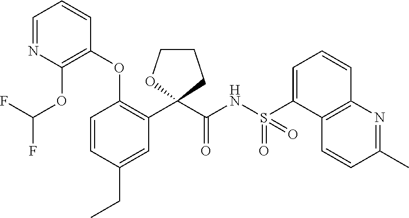 [SMILES: CCc1ccc(Oc2cccnc2OC(F)F)c([C@]2(C(=O)NS(=O)(=O)c3cccc4nc(C)ccc34)CCCO2)c1] |
| 27 | 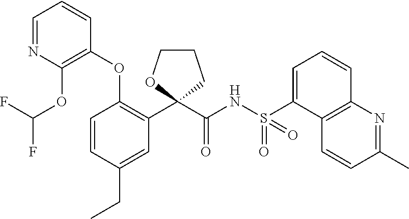 [SMILES: CCc1ccc(Oc2cccnc2OC(F)F)c([C@@]2(C(=O)NS(=O)(=O)c3cccc4nc(C)ccc34)CCCO2)c1] |
| 28 | 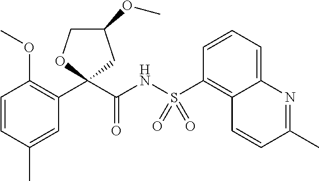 |
| 29 | 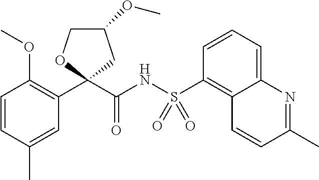 |
| 30 | 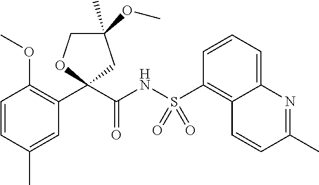 |
| 31 | 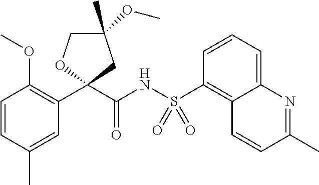 |
| 32 | 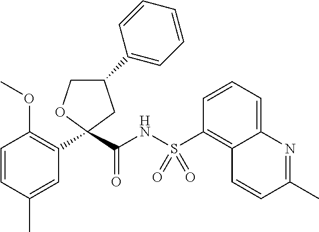 |
| 33 | 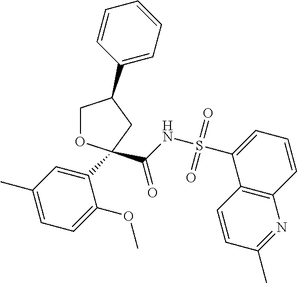 [SMILES: COc1ccc(C)cc1[C@@]1(C(=O)NS(=O)(=O)c2cccc3nc(C)ccc23)C[C@H](c2ccccc2)CO1] |
| 34 | 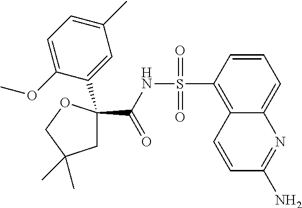 [SMILES: COc1ccc(C)cc1[C@]1(C(=O)NS(=O)(=O)c2cccc3nc(N)ccc23)CC(C)(C)CO1] |
| 35 | 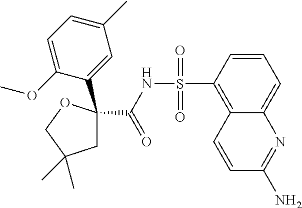 [SMILES: COc1ccc(C)cc1[C@@]1(C(=O)NS(=O)(=O)c2cccc3nc(N)ccc23)CC(C)(C)CO1] |
| 36 | 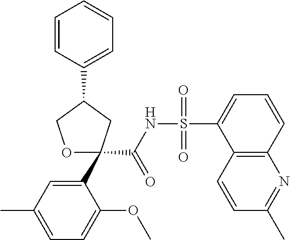 |
| 37 | 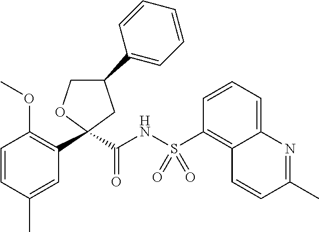 |
| 38 | 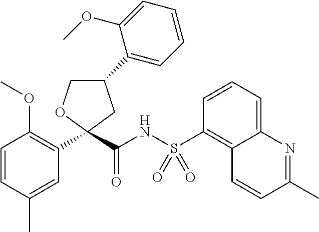 |
| 39 | 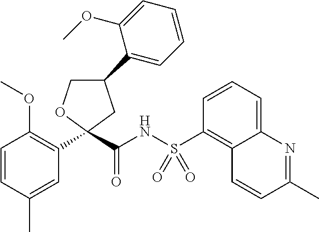 |
| 40 | 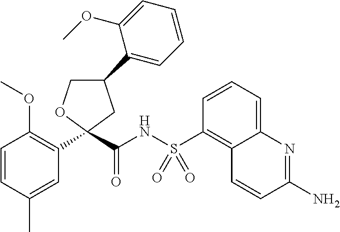 [SMILES: COc1ccccc1[C@@H]1CO[C@@](C(=O)NS(=O)(=O)c2cccc3nc(N)ccc23)(c2cc(C)ccc2OC)C1] |
| 41 | 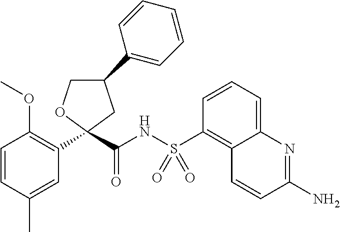 [SMILES: COc1ccc(C)cc1[C@@]1(C(=O)NS(=O)(=O)c2cccc3nc(N)ccc23)C[C@H](c2ccccc2)CO1] |
| 42 | 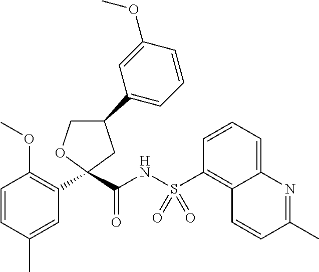 |
| 43 | 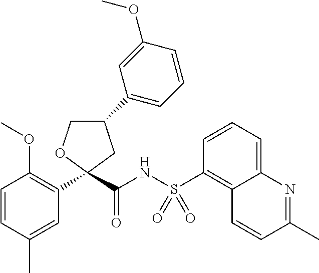 |
| 44 | 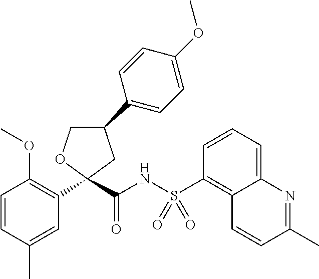 |
| 45 | 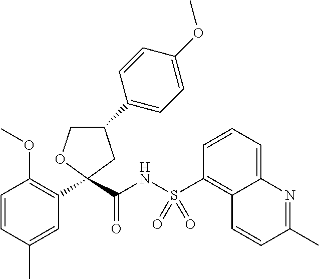 |

Compounds of Formula (I), Formula (II), or Formula (III) may be used in the form of pharmaceutically acceptable salts.

Compounds of Formula (I), Formula (II), or Formula (III) may contain either a basic or an acidic functionality, or both, and may be converted to a pharmaceutically acceptable salt, when desired, by using a suitable acid or base. The salts may be prepared in situ during the final isolation and purification of the compounds of the invention.

### Methods of Making Exemplary Compounds

The compounds of the invention can be better understood in connection with the following synthetic schemes and methods which illustrate a means by which the compounds can be prepared. The compounds of this invention can be prepared by a variety of synthetic procedures. Representative synthetic procedures are shown in, but not limited to, Schemes 1-2. The variables R¹, R², R³, m, and n are defined as detailed herein, e.g., in the Summary.

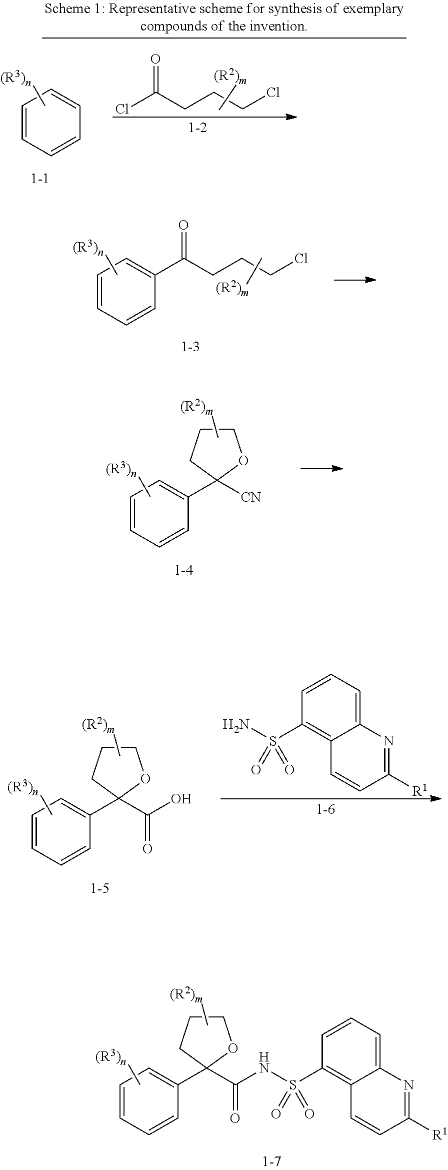

As shown in Scheme 1, compounds 1-7 can be prepared from compounds 1-1. Substituted or unsubstituted aryl compounds 1-1 can be acylated with acyl halides including compounds 1-2 or alternatively acid anhydrides under Friedel-Crafts conditions using a Lewis acid including, for example, AlCl₃ at reduced temperature to afford ketones 1-3. Keto-halides 1-3 can be cyclized to tetrahydrofurans 1-4 via treatment with KCN at elevated temperature. Hydrolysis of the nitrile group of 1-4 with hydroxide including, for example, NaOH, at elevated temperature affords carboxylic acid 1-5 which can be coupled with sulfonamides 1-6 to afford compounds 1-7. Any suitable coupling conditions known to one skilled in the art can be used to affect the coupling of 1-5 and 1-6, including, for example, treatment with 1-(3-dimethylaminopropyl)-3-ethylcarbodiimide hydrochloride and 4-dimethylaminopyridine in a solvent including, for example, dichloromethane.

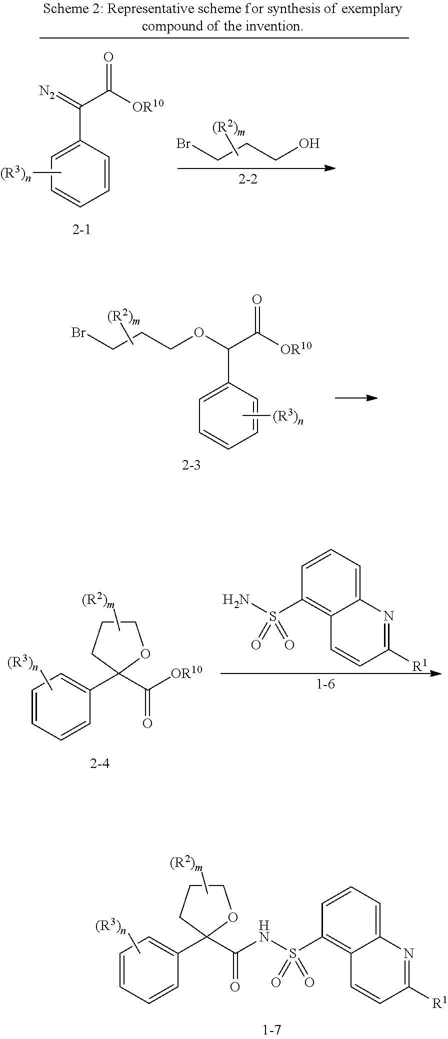

As shown in Scheme 2, compounds 1-7 can also be prepared from compound 2-1. Carbene insertion of compounds 2-1 (where R¹⁰ is alkyl or another suitable carboxylic acid protecting group) to alcohols 2-2 can be affected by a suitable catalyst including for example, Rh₂(OAc)₄ at reduced temperature in a suitable solvent including, for example dichloromethane. Cyclization of bromides 2-3 in the presence of a suitable base including for example, sodium bis(trimethylsilyl)amide at reduced temperature affords tetrahydrofurans 2-4 in which the ester group can be hydrolyzed to the corresponding carboxylic acid 2-4 (where R¹⁰ is hydrogen) by methods known to one skilled in the art including for example, treatment with KOH at elevated temperature. Coupling of compounds 2-4 where R¹⁰ is hydrogen with compounds 1-6 as disclosed in Scheme 1, affords compounds 1-7.

It can be appreciated that the synthetic schemes and specific examples as illustrated in the synthetic examples section are illustrative and are not to be read as limiting the scope of the invention as it is defined in the appended claims. All alternatives, modifications, and equivalents of the synthetic methods and specific examples are included within the scope of the claims.

Optimum reaction conditions and reaction times for each individual step can vary depending on the particular reactants employed and substituents present in the reactants used. Specific procedures are provided in the Examples section. Reactions can be worked up in the conventional manner, e.g. by eliminating the solvent from the residue and further purified according to methodologies generally known in the art such as, but not limited to, crystallization, distillation, extraction, trituration and chromatography. Unless otherwise described, the starting materials and reagents are either commercially available or can be prepared by one skilled in the art from standard organic chemical techniques, techniques that are analogous to the synthesis of known, structurally similar compounds, or techniques that are analogous to the above described schemes or the procedures described in the synthetic examples section.

Routine experimentations, including appropriate manipulation of the reaction conditions, reagents and sequence of the synthetic route, protection of any chemical functionality that is incompatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in T. Greene and P. Wuts, Protecting Groups in Organic Synthesis (3^{rd} ed.), John Wiley & Sons, NY (1999), which is incorporated herein by reference in its entirety. Synthesis of the compounds of the invention can be accomplished by methods analogous to those described in the synthetic schemes described hereinabove and in specific examples.

When an optically active form of a compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization or enzymatic resolution).

Similarly, when a pure geometric isomer of a compound is required, it can be prepared by carrying out one of the above procedures using a pure geometric isomer as a starting material, or by resolution of a mixture of the geometric isomers of the compound or intermediates using a standard procedure such as chromatographic separation.

### Pharmaceutical Compositions

When employed as a pharmaceutical, a compound of the invention is typically administered in the form of a pharmaceutical composition. Such composition may be prepared in a manner known in the pharmaceutical art and comprise a therapeutically effective amount of a compound of Formula (I), or a pharmaceutically acceptable salt thereof, together with a pharmaceutically acceptable carrier. The phrase “pharmaceutical composition” refers to a composition suitable for administration in medical or veterinary use.

The term “pharmaceutically acceptable carrier” as used herein, means a non-toxic, inert solid, semi-solid or liquid filler, diluent, encapsulating material or formulation auxiliary of any type.

In certain embodiments, a pharmaceutical composition is provided comprising a therapeutically effective amount of a compound of Formula (I), or a pharmaceutically acceptable salt thereof, in combination with a pharmaceutically acceptable carrier.

In certain embodiments, a pharmaceutical composition is provided comprising a therapeutically effective amount of a compound of Formula (I), or a pharmaceutically acceptable salt thereof, one or more potentiator, and one or more additional correctors.

### Method of Use

The compounds of Formula (I), or pharmaceutically acceptable salts thereof, and pharmaceutical compositions comprising a compound of Formula (I), or a pharmaceutically acceptable salt thereof, using any amount and any route of administration may be administered to a subject for the treatment or prevention of cystic fibrosis.

The term “administering” refers to the method of contacting a compound with a subject.

Compounds of the invention are useful as modulators of CFTR. Thus, the compounds and compositions are particularly useful for treating or lessening the severity or progression of a disease, disorder, or a condition where hyperactivity or inactivity of CFTR is involved. Accordingly, the invention provides a method for treating cystic fibrosis in a subject, wherein the method comprises the step of administering to said subject a therapeutically effective amount of a compound of formula (I) or a preferred embodiment thereof as set forth above, with or without a pharmaceutically acceptable carrier. Particularly, the method is for the treatment or prevention of cystic fibrosis. In a more particular embodiment, the cystic fibrosis is caused by a Class I, II, III, IV, V, and/or VI mutation.

In certain embodiments, the present invention provides compounds of the invention, or pharmaceutical compositions comprising a compound of the invention for use in medicine. In a particular embodiment, the present invention provides compounds of the invention, or a pharmaceutically acceptable salt thereof, or a pharmaceutical composition comprising a compound of the invention, for use in medicine. In a particular embodiment, the present invention provides compounds of the invention or pharmaceutical compositions comprising a compound of the invention, for use in the treatment of cystic fibrosis. In a more particular embodiment, the cystic fibrosis is caused by a Class I, II, III, IV, V, and/or VI mutation.

Certain embodiments are directed to the use of a compound according to formula (I) or a pharmaceutically acceptable salt thereof, in the preparation of a medicament. The medicament optionally can comprise one or more additional therapeutic agents. In some embodiments, the medicament is for use in the treatment of cystic fibrosis. In a more particular embodiment, the cystic fibrosis is caused by a Class I, II, III, IV, V, and/or VI mutation.

This invention also is directed to the use of a compound according to formula (I) or a pharmaceutically acceptable salt thereof, in the manufacture of a medicament for the treatment of cystic fibrosis. The medicament optionally can comprise one or more additional therapeutic agents. In a particular embodiment, the invention is directed to the use of a compound according to formula (I), or a pharmaceutically acceptable salt thereof, in the manufacture of a medicament for the treatment of cystic fibrosis. In a more particular embodiment, the cystic fibrosis is caused by a Class I, II, III, IV, V, and/or VI mutation.

In certain embodiments, the present invention provides pharmaceutical compositions comprising a compound of the invention, or a pharmaceutically acceptable salt thereof, and one or more additional therapeutic agents. In certain embodiments, the present invention provides pharmaceutical compositions comprising a compound of the invention, or a pharmaceutically acceptable salt thereof, and one or more additional therapeutic agents wherein the additional therapeutic agents are selected from the group consisting of CFTR modulators and CFTR amplifiers. In certain embodiments, the present invention provides pharmaceutical compositions comprising a compound of the invention, or a pharmaceutically acceptable salt thereof, and one or more additional therapeutic agents wherein the additional therapeutic agents are CFTR modulators.

The present compounds or pharmaceutically acceptable salts thereof may be administered as the sole active agent or it may be co-administered with other therapeutic agents, including other compounds or a pharmaceutically acceptable salt thereof that demonstrate the same or a similar therapeutic activity and that are determined to be safe and efficacious for such combined administration. The present compounds may be co-administered to a subject. The term “co-administered” means the administration of two or more different therapeutic agents to a subject in a single pharmaceutical composition or in separate pharmaceutical compositions. Thus, co-administration involves administration at the same time of a single pharmaceutical composition comprising two or more therapeutic agents or administration of two or more different compositions to the same subject at the same or different times.

The compounds of the invention or pharmaceutically acceptable salts thereof may be co-administered with a therapeutically effective amount of one or more additional therapeutic agents to treat a CFTR mediated disease, where examples of the therapeutic agents include, but are not limited to antibiotics (for example, aminoglycosides, colistin, aztreonam, ciprofloxacin, and azithromycin), expectorants (for example, hypertonic saline, acetylcysteine, dornase alfa, and denufosol), pancreatic enzyme supplements (for example, pancreatin, and pancrelipase), epithelial sodium channel blocker (ENaC) inhibitors, CFTR modulators (for example, CFTR potentiators, CFTR correctors), and CFTR amplifiers.

In certain embodiments, the compounds of the invention or pharmaceutically acceptable salts thereof may be co-administered with one or two CFTR modulators and one CFTR amplifier. In certain embodiments, the compounds of the invention or pharmaceutically acceptable salts thereof may be co-administered with one potentiator, and one or more correctors. In certain embodiments, the compounds of the invention or pharmaceutically acceptable salts thereof may be co-administered with one potentiator, one or more correctors, and one CFTR amplifier. In certain embodiments, the compounds of the invention or pharmaceutically acceptable salts thereof may be co-administered with one or more CFTR modulators. In certain embodiments, the compounds of the invention or pharmaceutically acceptable salts thereof may be co-administered with one CFTR modulator. In certain embodiments, the compounds of the invention or pharmaceutically acceptable salts thereof may be co-administered with two CFTR modulators. In certain embodiments, the compounds of the invention or pharmaceutically acceptable salts thereof may be co-administered with three CFTR modulators. In certain embodiments, the compounds of the invention or pharmaceutically acceptable salts thereof may be co-administered with one potentiator and one or more correctors. In certain embodiments, the compounds of the invention or pharmaceutically acceptable salts thereof may be co-administered with one potentiator and two correctors. In certain embodiments, the compounds of the invention or pharmaceutically acceptable salts thereof may be co-administered with one potentiator. In certain embodiments, the compounds of the invention or pharmaceutically acceptable salts thereof may be co-administered with one or more correctors. In certain embodiments, the compounds of the invention or pharmaceutically acceptable salts thereof may be co-administered with one corrector. In certain embodiments, the compounds of the invention or pharmaceutically acceptable salts thereof may be co-administered two correctors. In certain embodiments, the compounds of the invention or pharmaceutically acceptable salts thereof may be co-administered with one or more correctors, and one amplifier. In certain embodiments, the compounds of the invention or pharmaceutically acceptable salts thereof may be co-administered with one corrector, and one amplifier. In certain embodiments, the compounds of the invention or pharmaceutically acceptable salts thereof may be co-administered with two correctors, and one amplifier. In certain embodiments, the compounds of the invention or pharmaceutically acceptable salts thereof may be co-administered with one corrector. In certain embodiments, the compounds of the invention or pharmaceutically acceptable salts thereof may be co-administered with two correctors.

Examples of CFTR potentiators include, but are not limited to, Ivacaftor (VX-770), ABBV-2451, 4-amino-7-{[1-(2-fluorophenyl)-1H-pyrazol-4-yl]methyl}-5-[2-(trifluoromethyl)pyrimidin-5-yl]-7H-pyrrolo[2,3-d]pyrimidine-6-carbonitrile, GLPG1837, VX-561, NVS-QBW251, FD1860293, PTI-808, N-(3-carbamoyl-5,5,7,7-tetramethyl-5,7-dihydro-4H-thieno[2,3-c]pyran-2-yl)-1H-pyrazole-5-carboxamide, 3-amino-N-[(2S)-2-hydroxypropyl]-5-{[4-(trifluoromethoxy)phenyl]sulfonyl}pyridine-2-carboxamide and 4-amino-7-{[1-(2-fluorophenyl)-1H-pyrazol-4-yl]methyl}-5-[2-(trifluoromethyl)pyrimidin-5-yl]-7H-pyrrolo[2,3-d]pyrimidine-6-carbonitrile. Examples of potentiators are also disclosed in publications: WO2005120497, WO2008147952, WO2009076593, WO2010048573, WO2006002421, WO2008147952, WO2011072241, WO2011113894, WO2013038373, WO2013038378, WO2013038381, WO2013038386, WO2013038390, WO2014/180562, WO2015018823, WO2016193812 WO2017208115 and WO2018094237.

In certain embodiments, the potentiator is selected from the group consisting of
Ivacaftor (VX-770, N-(2,4-di-tert-butyl-5-hydroxyphenyl)-4-oxo-1,4-dihydroquinoline-3-carboxamide);
ABBV-2451;
GLPG1837;
VX-561;
NVS-QBW251;
FD1860293;
PTI-808;
2-(2-fluorobenzamido)-5,5,7,7-tetramethyl-5,7-dihydro-4H-thieno[2,3-c]pyran-3-carboxamide;
N-(3-carbamoyl-5,5,7,7-tetramethyl-5,7-dihydro-4H-thieno[2,3-c]pyran-2-yl)-1H-pyrazole-5-carboxamide;
2-(2-hydroxybenzamido)-5,5,7,7-tetramethyl-5,7-dihydro-4H-thieno[2,3-c]pyran-3-carboxamide;
2-(1-hydroxycyclopropanecarboxamido)-5,5,7,7-tetramethyl-5,7-dihydro-4H-thieno[2,3-c]pyran-3-carboxamide;
5,5,7,7-tetramethyl-2-(2-(trifluoromethyl)benzamido)-5,7-dihydro-4H-thieno[2,3-c]pyran-3-carboxamide;
2-(2-hydroxy-2-methylpropanamido)-5,5,7,7-tetramethyl-5,7-dihydro-4H-thieno[2,3-c]pyran-3-carboxamide;
2-(1-(hydroxymethyl)cyclopropanecarboxamido)-5,5,7,7-tetramethyl-5,7-dihydro-4H-thieno[2,3-c]pyran-3-carboxamide;
2-(3-hydroxy-2,2-dimethylpropanamido)-5,5,7,7-tetramethyl-5,7-dihydro-4H-thieno[2,3-c]pyran-3-carboxamide;
N-(3-carbamoyl-5,5,7,7-tetramethyl-5,7-dihydro-4H-thieno[2,3-c]pyran-2-yl)-5-methyl-1H-pyrazole-3-carboxamide;
N-(3-carbamoyl-5,5,7,7-tetramethyl-5,7-dihydro-4H-thieno[2,3-c]pyran-2-yl)-5-cyclopropyl-1H-pyrazole-3-carboxamide;
N-(3-carbamoyl-5,5,7,7-tetramethyl-5,7-dihydro-4H-thieno[2,3-c]pyran-2-yl)-5-isopropyl-1H-pyrazole-3-carboxamide;
N-(3-carbamoyl-5,5,7,7-tetramethyl-5,7-dihydro-4H-thieno[2,3-c]pyran-2-yl)-5-(trifluoromethyl)-1H-pyrazole-3-carboxamide;
5-tert-butyl-N-(3-carbamoyl-5,5,7,7-tetramethyl-5,7-dihydro-4H-thieno[2,3-c]pyran-2-yl)-1H-pyrazole-3-carboxamide;
N-(3-carbamoyl-5,5,7,7-tetramethyl-5,7-dihydro-4H-thieno[2,3-c]pyran-2-yl)-5-ethyl-1H-pyrazole-3-carboxamide;
N-(3-carbamoyl-5,5,7,7-tetramethyl-5,7-dihydro-4H-thieno[2,3-c]pyran-2-yl)-3-ethyl-4-methyl-1H-pyrazole-5-carboxamide;
2-(2-hydroxypropanamido)-5,5,7,7-tetramethyl-5,7-dihydro-4H-thieno[2,3-c]pyran-3-carboxamide;
N-(3-carbamoyl-5,5,7,7-tetramethyl-5,7-dihydro-4H-thieno[2,3-c]pyran-2-yl)-4-chloro-1H-pyrazole-3-carboxamide;
N-(3-carbamoyl-5,5,7,7-tetramethyl-5,7-dihydro-4H-thieno[2,3-c]pyran-2-yl)-1,4,6,7-tetrahydropyrano[4,3-c]pyrazole-3-carboxamide;
4-bromo-N-(3-carbamoyl-5,5,7,7-tetramethyl-5,7-dihydro-4H-thieno[2,3-c]pyran-2-yl)-1H-pyrazole-3-carboxamide;
N-(3-carbamoyl-5,5,7,7-tetramethyl-5,7-dihydro-4H-thieno[2,3-c]pyran-2-yl)-4-chloro-5-methyl-1H-pyrazole-3-carboxamide;
N-(3-carbamoyl-5,5,7,7-tetramethyl-5,7-dihydro-4H-thieno[2,3-c]pyran-2-yl)-4-methyl-1H-pyrazole-3-carboxamide;
2-(2-hydroxy-3,3-dimethylbutanamido)-5,5,7,7-tetramethyl-5,7-dihydro-4H-thieno[2,3-c]pyran-3-carboxamide;
2-[(2-hydroxy-4-methyl-pentanoyl)amino]-5,5,7,7-tetramethyl-4H-thieno[2,3-c]pyran-3-carboxamide;
5-(2-methoxy-ethoxy)-1H-pyrazole-3-carboxylic acid (3-carbamoyl-5,5,7,7-tetramethyl-4,7-dihydro-5H-thieno[2,3-c]pyran-2-yl)-amide;
N-(3-carbamoyl-5,5,7,7-tetramethyl-4H-thieno[2,3-c]pyran-2-yl)-4-(3-methoxypropyl)-1H-pyrazole-3-carboxamide;
N-(3-carbamoyl-5,5,7,7-tetramethyl-4H-thieno[2,3-c]pyran-2-yl)-4-(2-ethoxyethyl)-1H-pyrazole-3-carboxamide;
2-[[(2S)-2-hydroxy-3,3-dimethyl-butanoyl]amino]-5,5,7,7-tetramethyl-4H-thieno[2,3-c]pyran-3-carboxamide;
2-[[(2R)-2-hydroxy-3,3-dimethyl-butanoyl]amino]-5,5,7,7-tetramethyl-4H-thieno[2,3-c]pyran-3-carboxamide;
2-[(2-hydroxy-2,3,3-trimethyl-butanoyl)amino]-5,5,7,7-tetramethyl-4H-thieno[2,3-c]pyran-3-carboxamide;
[5-[(3-carbamoyl-5,5,7,7-tetramethyl-4H-thieno[2,3-c]pyran-2-yl)carbamoyl]pyrazol-1-yl]methyl dihydrogen phosphate;
[3-[(3-carbamoyl-5,5,7,7-tetramethyl-4H-thieno[2,3-c]pyran-2-yl)carbamoyl]pyrazol-1-yl]methyl dihydrogen phosphate;
N-(3-carbamoyl-5,5,7,7-tetramethyl-4H-thieno[2,3-c]pyran-2-yl)-4-(1,4-dioxan-2-yl)-1H-pyrazole-3-carboxamide;
5,5,7,7-tetramethyl-2-[[(2S)-3,3,3-trifluoro-2-hydroxy-2-methyl-propanoyl]amino]-4H-thieno[2,3-c]pyran-3-carboxamide;
2-[[(2S)-2-hydroxypropanoyl]amino]-5,5,7,7-tetramethyl-4H-thieno[2,3-c]pyran-3-carboxamide;
3-amino-N-(2-hydroxy-2-methylpropyl)-5-{[4-(trifluoromethoxy)phenyl]sulfonyl}pyridine-2-carboxamide;
3-amino-N-[(4-hydroxy-1-methylpiperidin-4-yl)methyl]-5-{[4-(trifluoromethoxy)phenyl]sulfonyl}pyridine-2-carboxamide;
3-amino-N-(3-hydroxy-2,2-dimethylpropyl)-5-{[4-(trifluoromethoxy)phenyl]sulfonyl}pyridine-2-carboxamide;
3-amino-5-[(4-fluorophenyl)sulfonyl]-N-[(1-hydroxycyclopropyl)methyl]pyridine-2-carboxamide;
3-amino-5-[(4-fluorophenyl)sulfonyl]-N-[(2R)-3,3,3-trifluoro-2-hydroxypropyl]pyridine-2-carboxamide;
3-amino-5-[(3-fluorophenyl)sulfonyl]-N-(2-hydroxy-2-methylpropyl)pyridine-2-carboxamide;
3-amino-N-[2-(cyclopropylamino)-2-oxoethyl]-5-{[4-(trifluoromethoxy)phenyl]sulfonyl}pyridine-2-carboxamide;
(3-amino-5-{[4-(trifluoromethoxy)phenyl]sulfonyl}pyridin-2-yl)(azetidin-1-yl)methanone;
(3-amino-5-{[4-(trifluoromethoxy)phenyl]sulfonyl}pyridin-2-yl)[3-(hydroxymethyl)azetidin-1-yl]methanone;
(3-amino-5-{[4-(trifluoromethoxy)phenyl]sulfonyl}pyridin-2-yl)(3-fluoroazetidin-1-yl)methanone;
3-amino-N-[(2R)-2-hydroxy-3-methoxypropyl]-5-f{[4-(trifluoromethyl)phenyl]sulfonyl}pyridine-2-carboxamide;
(3-amino-5-{[2-fluoro-4-(trifluoromethoxy)phenyl]sulfonyl}pyridin-2-yl)(3-hydroxyazetidin-1-yl)methanone;
(3-amino-5-{[2-(trifluoromethoxy)phenyl]sulfonyl}pyridin-2-yl)(3,3-difluoroazetidin-1-yl)methanone;
rac-3-amino-N-[(3R,4S)-4-hydroxytetrahydro-2H-pyran-3-yl]-5-{[2-(trifluoromethoxy)phenyl]sulfonyl}pyridine-2-carboxamide;
3-amino-5-[(4,4-difluoropiperidin-1-yl)sulfonyl]-N-(3,3,3-trifluoro-2-hydroxypropyl)pyridine-2-carboxamide;
(3-amino-5-f{[2-(trifluoromethoxy)phenyl]sulfonyl}pyridin-2-yl)[3-hydroxy-3-(trifluoromethyl)azetidin-1-yl]methanone;
3-amino-N-(2-hydroxy-4-methylpentyl)-5-f{[4-(trifluoromethoxy)phenyl]sulfonyl}pyridine-2-carboxamide;
(3-amino-5-{[4-(trifluoromethyl)phenyl]sulfonyl}pyridin-2-yl)(3-hydroxy-3-methylazetidin-1-yl)methanone;
3-amino-N-(3,3,3-trifluoro-2-hydroxypropyl)-5-{[4-(trifluoromethyl)piperidin-1-yl]sulfonyl}pyridine-2-carboxamide;
3-amino-N-[2-hydroxy-1-(4-methoxyphenyl)ethyl]-5-{[4-(trifluoromethoxy)phenyl]sulfonyl}pyridine-2-carboxamide;
3-amino-5-[(3,3-difluoroazetidin-1-yl)sulfonyl]-N-(3,3,3-trifluoro-2-hydroxypropyl)pyridine-2-carboxamide;
3-amino-5-f{[2-fluoro-4-(trifluoromethyl)phenyl]sulfonyl}-N-[(2S)-2-hydroxypropyl]pyridine-2-carboxamide;
3-amino-5-f{[2-fluoro-4-(trifluoromethyl)phenyl]sulfonyl}-N-[(2R)-2-hydroxy-3-methoxypropyl]pyridine-2-carboxamide;
3-amino-N-[2-oxo-2-(propan-2-ylamino)ethyl]-5-f{[4-(trifluoromethyl)phenyl]sulfonyl}pyridine-2-carboxamide;
(3-amino-5-{[4-(trifluoromethyl)phenyl]sulfonyl}pyridin-2-yl)[3-hydroxy-3-(trifluoromethyl)azetidin-1-yl]methanone;
3-amino-5-f{[2-fluoro-4-(trifluoromethyl)phenyl]sulfonyl}-N-[(3R)-tetrahydrofuran-3-ylmethyl]pyridine-2-carboxamide;
(3-amino-5-f{[2-fluoro-4-(trifluoromethyl)phenyl]sulfonyl}pyridin-2-yl)[3-hydroxy-3-(trifluoromethyl)azetidin-1-yl]methanone;
3-amino-5-f{[2-fluoro-4-(trifluoromethyl)phenyl]sulfonyl}-N-[(3S)-tetrahydrofuran-3-ylmethyl]pyridine-2-carboxamide;
3-amino-5-{[2-fluoro-4-(trifluoromethoxy)phenyl]sulfonyl}-N-[(3S)-tetrahydrofuran-3-ylmethyl]pyridine-2-carboxamide;
3-amino-N-[2-hydroxy-3-(2,2,2-trifluoroethoxy)propyl]-5-{[4-(trifluoromethyl)phenyl]sulfonyl}pyridine-2-carboxamide;
3-amino-N-(3-tert-butoxy-2-hydroxypropyl)-5-{[2-fluoro-4-(trifluoromethyl)phenyl]sulfonyl}pyridine-2-carboxamide;
[3-amino-5-(phenylsulfonyl)pyridin-2-yl][3-hydroxy-3-(trifluoromethyl)azetidin-1-yl]methanone;
{3-amino-5-[(3-fluorophenyl)sulfonyl]pyridin-2-yl}[3-hydroxy-3-(trifluoromethyl)azetidin-1-yl]methanone; and
3-amino-N-[(2S)-2-hydroxypropyl]-5-{[4-(trifluoromethoxy)phenyl]sulfonyl}pyridine-2-carboxamide.

Non-limiting examples of correctors include Lumacaftor (VX-809), 1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanecarboxamide (VX-661, tezacaftor), VX-983, ABV-2222, GLPG2665, ABBV-2737, ABBV-2851, ABBV-3221, 1-{5-cyclopropyl-2-[(propan-2-yl)oxy]pyridin-3-yl}-N-(2-methylquinoline-5-sulfonyl)cyclopropane-1-carboxamide, 1-(5-ethyl-2-{[(2R)-1-methoxypropan-2-yl]oxy}phenyl)-N-(2-methylquinoline-5-sulfonyl)cyclopropane-1-carboxamide, 1-{5-ethyl-2-[(propan-2-yl)oxy]pyridin-3-yl}-N-(2-methylquinoline-5-sulfonyl)cyclopropane-1-carboxamide, PTI-801, VX-152, VX-440, VX-659 (bamocaftor), VX-445 (elexacaftor), VX-121, FDL169, FDL304, FD2052160, and FD2035659. Examples of correctors are also disclosed in U.S. Pat. Nos. 9,642,831, 9,567,322, 9,840,513, 10,118,916, 9,796,711, 9,890,158, 10,399,940, and 9,981,910.

In certain embodiments, the corrector(s) can be selected from the group consisting of

Lumacaftor (VX-809);
1-(2,2-difluoro-1,3-benzodioxol-5-yl)-N-{1-[(2R)-2,3-dihydroxypropyl]-6-fluoro-2-(1-hydroxy-2-methylpropan-2-yl)-1H-indol-5-yl}cyclopropanecarboxamide (VX-661, tezacaftor);
VX-983;
GLPG2665;
ABBV-2737;
ABBV-3221;
PTI-801;
VX-152;
VX-440;
VX-659;
VX-445 (elexacaftor)
FDL169
FDL304;
FD2052160;
FD2035659;
3-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-6-methyl-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-methyl-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-6-methoxy-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]cyclohexanecarboxylic acid;
3-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-methoxy-3,4-dihydro-2H-chromen-2-yl]cyclohexanecarboxylic acid;
3-[(2R,4R)-4-({[1-(2,2-difluoro-1,3-benzodioxol-5-yl)cyclopropyl]carbonyl}amino)-7-fluoro-3,4-dihydro-2H-chromen-2-yl]benzoic acid;
3-({3-[(2R,4R)-4-({[1-(2,2-difluoro-1,3-benzodioxol-5-yl)cyclopropyl]carbonyl}amino)-7-methyl-3,4-dihydro-2H-chromen-2-yl]benzoyl}amino)-1-methylcyclopentanecarboxylic acid;
3-[(2R,4R)-4-({[1-(2,2-difluoro-1,3-benzodioxol-5-yl)cyclopropyl]carbonyl}amino)-7-methyl-3,4-dihydro-2H-chromen-2-yl]-N-[(2R)-2,3-dihydroxypropyl]benzamide;
3-[(2R,4R)-4-({[1-(2,2-difluoro-1,3-benzodioxol-5-yl)cyclopropyl]carbonyl}amino)-7-(2-methoxyethoxy)-3,4-dihydro-2H-chromen-2-yl]benzoic acid;
3-[(2R,4R)-7-(benzyloxy)-4-({[1-(2,2-difluoro-1,3-benzodioxol-5-yl)cyclopropyl]carbonyl}amino)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-(2-fluoroethoxy)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-(trifluoromethyl)-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-7-(trifluoromethyl)-3,4-dihydro-2H-chromen-2-yl]cyclohexanecarboxylic acid;
4-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid;
3-[(2R,4R)-4-({[1-(2,2-difluoro-1,3-benzodioxol-5-yl)cyclopropyl]carbonyl}amino)-8-fluoro-3,4-dihydro-2H-chromen-2-yl]benzoic acid;
4-[(2R,4R)-4-({[1-(2,2-difluoro-1,3-benzodioxol-5-yl)cyclopropyl]carbonyl}amino)-3,4-dihydro-2H-chromen-2-yl]benzoic acid;
4-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]benzoic acid;
rac-3-[(2R,4S)-4-({[1-(2,2-difluoro-1,3-benzodioxol-5-yl)cyclopropyl]carbonyl}amino)tetrahydro-2H-pyran-2-yl]benzoic acid;
rac-4-[(2R,4S)-4-({[1-(2,2-difluoro-1,3-benzodioxol-5-yl)cyclopropyl]carbonyl}amino)tetrahydro-2H-pyran-2-yl]benzoic acid;
3-[(2S,4R)-4-({[1-(2,2-difluoro-1,3-benzodioxol-5-yl)cyclopropyl]carbonyl}amino)tetrahydro-2H-pyran-2-yl]benzoic acid;
3-[(2R,4S)-4-({[1-(2,2-difluoro-1,3-benzodioxol-5-yl)cyclopropyl]carbonyl}amino)tetrahydro-2H-pyran-2-yl]benzoic acid;
rac-3-[(2R,4S,6S)-4-({[1-(2,2-difluoro-1,3-benzodioxol-5-yl)cyclopropyl]carbonyl}amino)-6-phenyltetrahydro-2H-pyran-2-yl]benzoic acid;
3-[(2S,4R,6R)-4-({[1-(2,2-difluoro-1,3-benzodioxol-5-yl)cyclopropyl]carbonyl}amino)-6-phenyltetrahydro-2H-pyran-2-yl]benzoic acid;
3-[(2R,4S,6S)-4-({[1-(2,2-difluoro-1,3-benzodioxol-5-yl)cyclopropyl]carbonyl}amino)-6-phenyltetrahydro-2H-pyran-2-yl]benzoic acid;
4-[(2R,4S)-4-({[1-(2,2-difluoro-1,3-benzodioxol-5-yl)cyclopropyl]carbonyl}amino)tetrahydro-2H-pyran-2-yl]benzoic acid;
4-[6-(4-cyanopiperidin-1-yl)pyridin-3-yl]-3-cyclobutyl-N-(methanesulfonyl)-1-phenyl-1H-pyrazolo[3,4-b]pyridine-6-carboxamide;
3-cyclobutyl-N-(methanesulfonyl)-4-[4-(methoxymethyl)piperidin-1-yl]-1-phenyl-1H-pyrazolo[3,4-b]pyridine-6-carboxamide;
4-[6-(4-cyanopiperidin-1-yl)pyridin-3-yl]-3-cyclobutyl-N-(methanesulfonyl)-1-[2-(morpholin-4-yl)pyridin-4-yl]-1H-pyrazolo[3,4-b]pyridine-6-carboxamide;
N-(methanesulfonyl)-4-[4-(methoxymethyl)piperidin-1-yl]-1-[2-(morpholin-4-yl)pyridin-4-yl]-3-(propan-2-yl)-1H-pyrazolo[3,4-b]pyridine-6-carboxamide;
3-cyclobutyl-4-[4-(methoxymethyl)piperidin-1-yl]-N-[2-(morpholin-4-yl)ethanesulfonyl]-1-phenyl-1H-pyrazolo[3,4-b]pyridine-6-carboxamide;
3-cyclobutyl-N-[2-(dimethylamino)ethanesulfonyl]-4-[4-(methoxymethyl)piperidin-1-yl]-1-phenyl-1H-pyrazolo[3,4-b]pyridine-6-carboxamide;
1-(4-fluorophenyl)-N-(methanesulfonyl)-4-(1′-methyl[4,4′-bipiperidin]-1-yl)-3-(propan-2-yl)-1H-pyrazolo[3,4-b]pyridine-6-carboxamide;
3-cyclobutyl-N-(methanesulfonyl)-4-{4-[2-(morpholin-4-yl)ethyl]piperidin-1-yl}-1-phenyl-1H-pyrazolo[3,4-b]pyridine-6-carboxamide;
3-cyclobutyl-4-[4-(methoxymethyl)piperidin-1-yl]-N-(oxolane-3-sulfonyl)-1-phenyl-1H-pyrazolo[3,4-b]pyridine-6-carboxamide;
3-cyclobutyl-N-(dimethylsulfamoyl)-1-(4-fluorophenyl)-4-(4-methoxy[1,4′-bipiperidin]-1′-yl)-1H-pyrazolo[3,4-b]pyridine-6-carboxamide;
3-cyclobutyl-N-(morpholine-4-sulfonyl)-4-[4-(morpholin-4-yl)piperidin-1-yl]-1-phenyl-1H-pyrazolo[3,4-b]pyridine-6-carboxamide;
3-cyclobutyl-N-(morpholine-4-sulfonyl)-1-phenyl-4-{4-[(pyrrolidin-1-yl)methyl]piperidin-1-yl}-1H-pyrazolo[3,4-b]pyridine-6-carboxamide;
3-cyclobutyl-N-(methanesulfonyl)-4-[4-(morpholin-4-yl)piperidin-1-yl]-1-phenyl-1H-pyrazolo[3,4-b]pyridine-6-carboxamide;
3-cyclobutyl-4-[4-(morpholin-4-yl)piperidin-1-yl]-1-phenyl-1H-pyrazolo[3,4-b]pyridine-6-carboxylic acid;
3-cyclobutyl-1-phenyl-4-{4-[(pyrrolidin-1-yl)methyl]piperidin-1-yl}-1H-pyrazolo[3,4-b]pyridine-6-carboxylic acid;
5-[(2R,4R)-4-{[(7R)-2,2-difluoro-7-methyl-6,7-dihydro-2H-furo[2,3-f][1,3]benzodioxole-7-carbonyl]amino}-7-methoxy-3,4-dihydro-2H-1-benzopyran-2-yl]pyrazine-2-carboxylic acid;
6-[(2R,4R)-4-{[(7R)-2,2-difluoro-7-methyl-6,7-dihydro-2H-furo[2,3-f][1,3]benzodioxole-7-carbonyl]amino}-7-(trifluoromethoxy)-3,4-dihydro-2H-1-benzopyran-2-yl]pyridine-3-carboxylic acid;
trans-4-[(2S,4S)-4-{[(7R)-2,2-difluoro-7-methyl-6,7-dihydro-2H-furo[2,3-f][1,3]benzodioxole-7-carbonyl]amino}-7-(trifluoromethoxy)-3,4-dihydro-2H-1-benzopyran-2-yl]cyclohexane-1-carboxylic acid;
6-[(2R,4R)-7-(difluoromethoxy)-4-{[(7R)-2,2-difluoro-7-methyl-6,7-dihydro-2H-furo[2,3-f][1,3]benzodioxole-7-carbonyl]amino}-3,4-dihydro-2H-1-benzopyran-2-yl]pyridine-3-carboxylic acid;
trans-4-[(2S,4S)-4-{[(7R)-2,2-difluoro-7-methyl-6,7-dihydro-2H-furo[2,3-f][1,3]benzodioxole-7-carbonyl]amino}-7-methoxy-3,4-dihydro-2H-1-benzopyran-2-yl]cyclohexane-1-carboxylic acid;
ethyl trans-4-[(2S,4S)-7-(difluoromethoxy)-4-{[(7R)-2,2-difluoro-7-methyl-6,7-dihydro-2H-furo[2,3-f][1,3]benzodioxole-7-carbonyl]amino}-3,4-dihydro-2H-1-benzopyran-2-yl]cyclohexane-1-carboxylate;
cis-4-[(2R,4R)-4-{[(7R)-2,2-difluoro-7-methyl-6,7-dihydro-2H-furo[2,3-f][1,3]benzodioxole-7-carbonyl]amino}-7-(trifluoromethoxy)-3,4-dihydro-2H-1-benzopyran-2-yl]cyclohexane-1-carboxylic acid;
trans-4-[(2S,4S)-7-(difluoromethoxy)-4-{[(7R)-2,2-difluoro-7-methyl-6,7-dihydro-2H-furo[2,3-f][1,3]benzodioxole-7-carbonyl]amino}-3,4-dihydro-2H-1-benzopyran-2-yl]cyclohexane-1-carboxylic acid;
1-[(2R,4R)-4-{[(7R)-2,2-difluoro-7-methyl-6,7-dihydro-2H-furo[2,3-f][1,3]benzodioxole-7-carbonyl]amino}-7-(trifluoromethoxy)-3,4-dihydro-2H-1-benzopyran-2-yl]cyclopropane-1-carboxylic acid;
trans-4-[(2R,4R)-4-{[(5S)-2,2-difluoro-5-methyl-6,7-dihydro-2H,5H-indeno[5,6-d][1,3]dioxole-5-carbonyl]amino}-7-(trifluoromethoxy)-3,4-dihydro-2H-1-benzopyran-2-yl]cyclohexane-1-carboxylic acid;
trans-4-[(2R,4R)-4-{[(5S)-2,2-difluoro-5-methyl-6,7-dihydro-2H,5H-indeno[5,6-d][1,3]dioxole-5-carbonyl]amino}-7-methoxy-3,4-dihydro-2H-1-benzopyran-2-yl]cyclohexane-1-carboxylic acid;
trans-4-[(2R,4R)-4-{[(7R)-2,2-difluoro-7-methyl-6,7-dihydro-2H-furo[2,3-f][1,3]benzodioxole-7-carbonyl]amino}-7-methoxy-3,4-dihydro-2H-1-benzopyran-2-yl]cyclohexane-1-carboxylic acid;
trans-4-[(2R,4R)-7-(difluoromethoxy)-4-{[(7R)-2,2-difluoro-7-methyl-6,7-dihydro-2H-furo[2,3-f][1,3]benzodioxole-7-carbonyl]amino}-3,4-dihydro-2H-1-benzopyran-2-yl]cyclohexane-1-carboxylic acid;
trans-4-[(2R,4R)-4-{[(7R)-2,2-difluoro-7-methyl-6,7-dihydro-2H-furo[2,3-f][1,3]benzodioxole-7-carbonyl]amino}-7-(trifluoromethoxy)-3,4-dihydro-2H-1-benzopyran-2-yl]cyclohexane-1-carboxylic acid;
4-{(2R,4R)-4-[2-(2,2-difluoro-2H-1,3-benzodioxol-5-yl)-2-methylpropanamido]-7-methoxy-3,4-dihydro-2H-1-benzopyran-2-yl}benzoic acid;
4-[(2R,4R)-4-{[1-(3,4-dichlorophenyl)cyclopropane-1-carbonyl]amino}-7-methoxy-3,4-dihydro-2H-1-benzopyran-2-yl]benzoic acid;
4-[(2R,4R)-4-{[1-(4-bromophenyl)cyclopropane-1-carbonyl]amino}-7-methoxy-3,4-dihydro-2H-1-benzopyran-2-yl]benzoic acid;
4-[(2R,4R)-7-methoxy-4-({1-[4-(trifluoromethyl)phenyl]cyclopropane-1-carbonyl}amino)-3,4-dihydro-2H-1-benzopyran-2-yl]benzoic acid;
4-[(2R,4R)-7-methoxy-4-{[1-(4-methylphenyl)cyclopropane-1-carbonyl]amino}-3,4-dihydro-2H-1-benzopyran-2-yl]benzoic acid;
4-{(2R,4R)-4-[(1,5-dimethyl-2,3-dihydro-1H-indene-1-carbonyl)amino]-7-methoxy-3,4-dihydro-2H-1-benzopyran-2-yl}benzoic acid;
3-[(2R,4R)-4-{[(1S)-1,5-dimethyl-2,3-dihydro-1H-indene-1-carbonyl]amino}-7-methoxy-3,4-dihydro-2H-1-benzopyran-2-yl]benzoic acid;
4-[(2R,4R)-4-{[(1S)-1,5-dimethyl-2,3-dihydro-1H-indene-1-carbonyl]amino}-7-methoxy-3,4-dihydro-2H-1-benzopyran-2-yl]benzoic acid;
trans-4-[(2R,4R)-4-{[1-(2,2-difluoro-2H-1,3-benzodioxol-5-yl)cyclopropane-1-carbonyl]amino}-7-methoxy-3,4-dihydro-2H-1-benzopyran-2-yl]cyclohexane-1-carboxylic acid;
trans-4-[(2R,4R)-4-{[1-(2,2-difluoro-2H-1,3-benzodioxol-5-yl)cyclopropane-1-carbonyl]amino}-7-(trifluoromethoxy)-3,4-dihydro-2H-1-benzopyran-2-yl]cyclohexane-1-carboxylic acid; and
4-[(2R,4R)-4-{[1-(2,2-difluoro-2H-1,3-benzodioxol-5-yl)cyclopropane-1-carbonyl]amino}-7-(difluoromethoxy)-3,4-dihydro-2H-1-benzopyran-2-yl]cyclohexane-1-carboxylic acid.

In certain embodiments, the additional therapeutic agent is a CFTR amplifier. CFTR amplifiers enhance the effect of known CFTR modulators, such as potentiators and correctors. Examples of CFTR amplifiers include PTI130 and PTI-428. Examples of amplifiers are also disclosed in International Patent Publication Nos.: WO2015138909 and WO2015138934.

In certain embodiments, the additional therapeutic agent is a CFTR stabilizer. CFTR stabilizers enhance the stability of corrected CFTR that has been treated with a corrector, corrector/potentiator or other CFTR modulator combination(s). An example of a CFTR stabilizer is cavosonstat (N91115). Examples of stabilizers are also disclosed in International Patent Publication No.: WO2012048181.

In certain embodiments, the additional therapeutic agent is an agent that reduces the activity of the epithelial sodium channel blocker (ENaC) either directly by blocking the channel or indirectly by modulation of proteases that lead to an increase in ENaC activity (e.g., serine proteases, channel-activating proteases). Exemplary of such agents include camostat (a trypsin-like protease inhibitor), QAU145, 552-02, GS-9411, INO-4995, Aerolytic, amiloride, VX-371 and ETD001. Additional agents that reduce the activity of the epithelial sodium channel blocker (ENaC) can be found, for example, in International Patent Publication Nos.: WO2009074575 and WO2013043720; and U.S. Pat. No. 8,999,976.

In certain embodiments, the ENaC inhibitor is VX-371.

In certain embodiments, the ENaC inhibitor is SPX-101 (S18).

In certain embodiments, the ENac inhibitor is ETD001.

In certain embodiments, the additional therapeutic agent is a Transmembrane membrane 16A (TMEM16A) potentiator. TMEM16A potentiators enhance the flow of chloride across the lung cell membrane via calcium-activated TMEM16A channels present on the apical membrane of the epithelial cells. The increased chloride flow would result in increased mucus hydration. Examples of TMEME16A potentiators include ETD002. Examples of TMEM16A potentiators are also disclosed in International Patent Publication No.: WO2019145726.

In certain embodiments, a method for treating cystic fibrosis in a subject is provided, the method comprising administering a therapeutically effective amount of a compound of formula (I), or a pharmaceutically acceptable salt thereof, to a subject in need thereof.

This invention also is directed to kits that comprise one or more compounds and/or salts of the invention, and, optionally, one or more additional therapeutic agents.

This invention also is directed to methods of use of the compounds, salts, compositions, and/or kits of the invention to, for example, modulate the Cystic Fibrosis Transmembrane Conductance Regulator (CFTR) protein, and treat a disease treatable by modulating the Cystic Fibrosis Transmembrane Conductance Regulator (CFTR) protein (including cystic fibrosis).

In certain embodiments, the present invention provides compounds of the invention, or pharmaceutical compositions comprising a compound of the invention, for use in medicine. In a particular embodiment, the present invention provides compounds of the invention, or pharmaceutical compositions comprising a compound of the invention, for use in the treatment of diseases or disorders as described herein above.

Certain embodiments are directed to the use of a compound according to Formula (I), or a pharmaceutically acceptable salt thereof in the preparation of a medicament. In some embodiments the medicament is for use in the treatment of diseases and disorders as described herein above.

This invention is also directed to the use of a compound according to Formula (I), or a pharmaceutically acceptable salt thereof in the manufacture of a medicament for the treatment of the diseases and disorders as described herein above.

### EXAMPLES

The following Examples may be used for illustrative purposes and should not be deemed to narrow the scope of the invention.

All reagents were of commercial grade and were used as received without further purification, unless otherwise stated. Commercially available anhydrous solvents were used for reactions conducted under inert atmosphere. Reagent grade solvents were used in all other cases, unless otherwise specified. Chemical shifts (δ) for ¹H NMR spectra were reported in parts per million (ppm) relative to tetramethylsilane (δ 0.00) or the appropriate residual solvent peak, i.e. CHCl₃ (δ 7.27), as internal reference.

The following abbreviations have the indicated meaning unless otherwise specified: NMR for nuclear magnetic resonance; s for singlet; br s for broad singlet; d for duplet or doublet; m for multiplet; t for triplet; q for quartet; LC/MS or LCMS for liquid chromatography-mass spectrometry; min for minute; mL for milliliter; μL for microliter; L for liter; g for gram; mg for milligram; mmol for millimoles; psi for pounds per square inch; HPLC for high pressure liquid chromatography; ppm for parts per million; APCI for atmospheric pressure chemical ionization; DCI for desorption chemical ionization; DSI for droplet spray ionization; ESI for electrospray ionization; RT for retention time; M for molarity (moles/liter); N for normality (equivalent/liter); ee for enantiomeric excess; and de for diastereomeric excess.

### Example 1

### (2S)-2-(2-ethoxy-5-methylphenyl)-N-(2-methylquinoline-5-sulfonyl)oxolane-2-carboxamide

### Example 1A

### 1-ethoxy-4-methylbenzene

A mixture of p-cresol (3.24 g, 30 mmol, Aldrich), potassium carbonate (6.22 g, 45.0 mmol), and iodoethane (3.0 mL, 37.5 mmol) in N,N-dimethylformamide (15.0 mL) was heated to 80° C. After 2 hours, the reaction was diluted with ethyl acetate and washed twice with water and once with brine. The organic phase was dried with MgSO₄, filtered, and concentrated under reduced pressure to afford the title compound (3.17 g, 23.36 mmol, 78% yield). ¹H NMR (500 MHz, CDCl₃) δ ppm 7.10-7.04 (m, 2H), 6.82-6.77 (m, 2H), 4.00 (q, J=7.0 Hz, 2H), 2.28 (d, J=0.7 Hz, 3H), 1.40 (t, J=7.0 Hz, 3H).

### Example 1B

### 4-chloro-1-(2-hydroxy-5-methylphenyl)butan-1-one

Example 1A (1.63 g, 12 mmol) was added dropwise to a suspension of aluminum chloride (2.00 g, 15.0 mmol) in dichloromethane (40.0 mL) at 0° C. After 5 minutes, 4-chlorobutanoyl chloride (1.69 mL, 15.0 mmol, Aldrich) was added dropwise, and the reaction was warmed to ambient temperature. After stirring for 3 hours, the reaction as poured into 1 M HCl (200 mL) and extracted twice with dichloromethane. The combined organic layers were then washed with water, washed with brine, dried with MgSO₄, filtered, and concentrated under reduced pressure. The crude residue was purified by flash chromatography (ISCO CombiFlash, 0-30% ethyl acetate/heptanes, 80 g RediSep® gold silica column) to afford the title compound (2.3 g, 10.8 mmol, 90% yield). ¹H NMR (400 MHz, CDCl₃) δ ppm 12.02 (s, 1H), 7.56 (dd, J=2.2, 0.9 Hz, 1H), 7.29 (dd, J=8.5, 2.2 Hz, 1H), 6.89 (d, J=8.5 Hz, 1H), 3.69 (t, J=6.2 Hz, 2H), 3.21 (t, J=7.0 Hz, 2H), 2.32 (s, 3H), 2.23 (tt, J=7.0, 6.1 Hz, 2H). MS(APCI+) m/z 213.5 (M+H)⁺.

### Example 1C

### 2-(2-hydroxy-5-methylphenyl)tetrahydrofuran-2-carbonitrile

Potassium cyanide (1.06 g, 16.2 mmol) was added to a solution of Example 1B (2.3 g, 10.8 mmol) in methanol (22 mL), and the resulting mixture was heated to 35° C. After heating for 48 hours, the reaction was diluted with ethyl acetate, washed twice with saturated NaHCO₃, and washed once with brine. The organic phase was dried with MgSO₄, filtered, and concentrated under reduced pressure. The crude residue was purified by flash chromatography (ISCO CombiFlash, 0-100% ethyl acetate/heptanes, 80 g RediSep® gold silica column) to afford the title compound (463 mg, 2.28 mmol, 21.1% yield). ¹H NMR (400 MHz, CDCL₃) δ ppm 7.18-7.01 (m, 2H), 6.85-6.72 (m, 1H), 4.37-4.06 (m, 2H), 2.93-2.76 (m, 1H), 2.45-2.12 (m, 6H). MS(APCI+) m/z 204.5 (M+H)⁺.

### Example 1D

### 2-(2-ethoxy-5-methylphenyl)tetrahydrofuran-2-carbonitrile

A mixture of Example 1C (460 mg, 2.263 mmol), iodoethane (229 μL, 2.83 mmol), and potassium carbonate (469 mg, 3.40 mmol) in N,N-dimethylformamide (4.5 mL) and was heated to 50° C. After stirring for 16 hours, the reaction was diluted with ethyl acetate, washed twice with water, and washed once with brine. The organic phase was dried with MgSO₄, filtered, and concentrated under reduced pressure. The crude residue was then purified by flash chromatography (ISCO CombiFlash, 0-30% ethyl acetate/heptanes, 40 g RediSep® gold silica column) to afford the title compound (324 mg, 1.40 mmol, 61.9% yield). ¹H NMR (500 MHz, CDCl₃) δ ppm 7.36 (dd, J=2.3, 0.8 Hz, 1H), 7.09 (ddq, J=8.2, 2.2, 0.7 Hz, 1H), 6.81 (d, J=8.3 Hz, 1H), 4.24-4.02 (m, 4H), 3.03-2.90 (m, 1H), 2.30 (d, J=0.7 Hz, 3H), 2.28-2.18 (m, 1H), 2.14-2.06 (m, 1H), 2.06-1.96 (m, 1H), 1.48 (t, J=7.0 Hz, 3H). MS(APCI+) m/z 205.0 (M-CN)⁺.

### Example 1E

### 2-(2-ethoxy-5-methylphenyl)tetrahydrofuran-2-carboxylic Acid

Sodium hydroxide (1.76 mL, 50% in water, 33.4 mmol) was added to a solution of Example 1D (772 mg, 3.34 mmol) in ethanol (6.7 mL), and the resulting mixture was heated to 90° C. After 72 hours, the reaction was acidified with 1 M HCl and extracted with ethyl acetate. The organic phase was dried with MgSO₄, filtered, and concentrated under reduced pressure to afford the title compound (784 mg, 3.13 mmol, 94% yield). ¹H NMR (400 MHz, dimethyl sulfoxide-d₆) δ ppm 12.16 (s, 1H), 7.21 (d, J=2.2 Hz, 1H), 6.99 (ddd, J=8.1, 2.3, 0.8 Hz, 1H), 6.82 (d, J=8.2 Hz, 1H), 4.09-3.78 (m, 4H), 2.94-2.77 (m, 1H), 2.24 (s, 3H), 2.05 1.90 (m, 1H), 1.86-1.63 (m, 2H), 1.24 (t, J=6.9 Hz, 3H). MS(APCI+) m/z 251.4 (M+H)⁺.

### Example 1F

### 2-(2-ethoxy-5-methylphenyl)-N-((2-methylquinolin-5-yl)sulfonyl)tetrahydrofuran-2-carboxamide

A mixture of Example 1E (34 mg, 0.136 mmol), 2-methylquinoline-5-sulfonamide (39.3 mg, 0.177 mmol, prepared as in WO2018154519 A1), 1-(3-dimethylaminopropyl)-3-ethylcarbodiimide hydrochloride (52.1 mg, 0.272 mmol), and 4-dimethylaminopyridine (21.57 mg, 0.177 mmol) in dichloromethane (1.8 mL) was stirred at ambient temperature. After 18 hours, the reaction was acidified with trifluoroacetic acid (52 μL, 0.68 mmol) and concentrated under reduced pressure. The crude residue was purified by reverse-phase HPLC (Waters Xbridge Prep C18 column, 42 mL/minute, 5-95% acetonitrile/0.1% trifluoroacetic acid in water) to afford the title compound (41.9 mg, 0.092 mmol, 67.9% yield). ¹H NMR (400 MHz, dimethyl sulfoxide-d₆) δ ppm 11.80 (s, 1H), 8.66 (d, J=8.9 Hz, 1H), 8.34-8.08 (m, 2H), 7.87 (t, J=7.9 Hz, 1H), 7.40 (d, J=8.8 Hz, 1H), 7.20 (d, J=2.3 Hz, 1H), 7.05-6.91 (m, 1H), 6.53 (d, J=8.2 Hz, 1H), 3.85 (q, J=7.0 Hz, 1H), 3.63 (dt, J=8.3, 6.8 Hz, 1H), 3.54 (dq, J=9.2, 6.9 Hz, 1H), 2.97 (t, J=7.9 Hz, 1H), 2.70 (s, 3H), 2.61 (dt, J=13.2, 6.9 Hz, 1H), 2.23 (s, 3H), 1.77-1.52 (m, 3H), 0.69 (t, J=6.9 Hz, 3H). MS(APCI+) m/z 455.4 (M+H)⁺.

### Example 1G

### (2S)-2-(2-ethoxy-5-methylphenyl)-N-(2-methylquinoline-5-sulfonyl)oxolane-2-carboxamide

The enantiomers of Example 1F (41 mg, 0.47 mmol) were separated by preparative chiral supercritical fluid chromatography (ChiralPak OZ-H column, 45% methanol/CO₂, 80 g/minute). The fractions containing the first eluting peak were concentrated under reduced pressure to afford the title compound (9.1 mg, 0.020 mmol, 22% yield). ¹H NMR (400 MHz, dimethyl sulfoxide-d₆) δ ppm 11.80 (s, 1H), 8.66 (d, J=8.9 Hz, 1H), 8.34-8.08 (m, 2H), 7.87 (t, J=7.9 Hz, 1H), 7.40 (d, J=8.8 Hz, 1H), 7.20 (d, J=2.3 Hz, 1H), 7.05-6.91 (m, 1H), 6.53 (d, J=8.2 Hz, 1H), 3.85 (q, J=7.0 Hz, 1H), 3.63 (dt, J=8.3, 6.8 Hz, 1H), 3.54 (dq, J=9.2, 6.9 Hz, 1H), 2.97 (t, J=7.9 Hz, 1H), 2.70 (s, 3H), 2.61 (dt, J=13.2, 6.9 Hz, 1H), 2.23 (s, 3H), 1.77-1.52 (m, 3H), 0.69 (t, J=6.9 Hz, 3H). MS(APCI+) m/z 455.4 (M+H)⁺.

### Example 2

### (2R)-2-(2-ethoxy-5-methylphenyl)-N-(2-methylquinoline-5-sulfonyl)oxolane-2-carboxamide

The enantiomers of Example 1F (41 mg, 0.47 mmol) were separated by preparative chiral supercritical fluid chromatography (ChiralPak OZ-H column, 45% methanol/CO₂, 80 g/minute). The fractions containing the second eluting peak were concentrated under reduced pressure to afford the title compound (9.8 mg, 0.022 mmol, 24% yield). ¹H NMR (400 MHz, dimethyl sulfoxide-d₆) δ ppm 11.80 (s, 1H), 8.66 (d, J=8.9 Hz, 1H), 8.34-8.08 (m, 2H), 7.87 (t, J=7.9 Hz, 1H), 7.40 (d, J=8.8 Hz, 1H), 7.20 (d, J=2.3 Hz, 1H), 7.05 6.91 (m, 1H), 6.53 (d, J=8.2 Hz, 1H), 3.85 (q, J=7.0 Hz, 1H), 3.63 (dt, J=8.3, 6.8 Hz, 1H), 3.54 (dq, J=9.2, 6.9 Hz, 1H), 2.97 (t, J=7.9 Hz, 1H), 2.70 (s, 3H), 2.61 (dt, J=13.2, 6.9 Hz, 1H), 2.23 (s, 3H), 1.77-1.52 (m, 3H), 0.69 (t, J=6.9 Hz, 3H). MS(APCI+) m/z 455.4 (M+H)⁺.

### Example 3

### 7-(2-methoxy-5-methylphenyl)-N-(2-methylquinoline-5-sulfonyl)-2,6-dioxaspiro[3.4]octane-7-carboxamide

### Example 3A

### methyl 2-(2-methoxy-5-methylphenyl)-2-oxoacetate

1-Methoxy-4-methylbenzene (6.30 mL, 50 mmol, Aldrich) was added to a suspension of aluminum chloride (8.00 g, 60.0 mmol) in dichloromethane (100 mL) at 0° C. After stirring for 10 minutes, methyl 2-chloro-2-oxoacetate (5.52 mL, 60.0 mmol, Aldrich) was added dropwise, and the reaction was allowed to slowly warm to ambient temperature. After 16 hours, the reaction was quenched with 1 M hydrochloric acid (200 mL) and vigorously stirred for 15 minutes. The layers were separated, and the organic phase was washed with saturated NaHCO₃, washed with brine, dried with MgSO₄, filtered, and concentrated under reduced pressure. The crude residue was purified by flash chromatography (ISCO CombiFlash, 0-30% ethyl acetate/heptanes, 120 g RediSep® gold silica column) to afford the title compound (8.9 g, 42.7 mmol, 85% yield). ¹H NMR (400 MHz, CDCl₃) δ ppm 7.67 (dd, J=2.3, 1.1 Hz, 1H), 7.43-7.34 (m, 1H), 6.89 (d, J=8.5 Hz, 1H), 3.91 (s, 3H), 3.84 (s, 3H), 2.33 (d, J=0.8 Hz, 3H). MS(APCI+) m/z 209.5 (M+H)⁺.

### Example 3B

### methyl 2-diazo-2-(2-methoxy-5-methylphenyl)acetate

A mixture of Example 3A (10.2 g, 49.0 mmol) and 4-methylbenzenesulfonohydrazide (9.12 g, 49.0 mmol, Aldrich) in toluene (100 mL) was heated at reflux with a Dean-Stark trap. After 16 hours, the reaction was concentrated under reduced pressure. Dichloromethane (100 mL) and triethylamine (8.19 mL, 58.8 mmol) were added to the resulting residue. After stirring at ambient temperature for 48 hours, the reaction was washed with saturated NaHCO₃, washed brine, dried over MgSO₄, and concentrated under reduced pressure. The crude residue was purified by flash chromatography (ISCO CombiFlash, 0-20% ethyl acetate/heptanes, 120 g RediSep® gold silica column) to afford the title compound (7.89 g, 35.8 mmol, 73.1% yield). ¹H NMR (500 MHz, CDCl₃) δ ppm 7.40-7.32 (m, 1H), 7.05 (ddt, J=8.4, 2.3, 0.7 Hz, 1H), 6.79 (d, J=8.4 Hz, 1H), 3.83 (s, 3H), 3.82 (s, 3H), 2.30 (t, J=0.7 Hz, 3H).

### Example 3C

### rac-methyl 2-((3-(bromomethyl)oxetan-3-yl)methoxy)-2-(2-methoxy-5-methylphenyl)acetate

A solution of Example 3B (300 mg, 1.362 mmol) in dichloromethane (5 mL) was added dropwise over 3 hours to a mixture of Rh₂(OAc)₄ (6.02 mg, 0.014 mmol) and (3-(bromomethyl)oxetan-3-yl)methanol (0.231 mL, 2.043 mmol) in dichloromethane (5.00 mL) at 0° C. The reaction was stirred at ambient temperature for an additional 1 hour, then loaded onto diatomaceous earth and purified by flash chromatography (ISCO CombiFlash, 0-100% ethyl acetate/heptanes) to afford the title compound (436 mg, 1.168 mmol, 86% yield). ¹H NMR (400 MHz, CDCl₃) δ ppm 7.15-7.07 (m, 2H), 6.87-6.74 (m, 1H), 5.34 (s, 1H), 4.50 (d, J=6.5 Hz, 1H), 4.46-4.41 (m, 3H), 3.91 (d, J=9.2 Hz, 1H), 3.83 (s, 3H), 3.83-3.76 (m, 3H), 3.73 (s, 3H), 2.28 (s, 3H). MS(ESI) m/z 373.2 (M+H)⁺ and 375.2 (M+H+2)⁺.

### Example 3D

### rac-methyl 7-(2-methoxy-5-methylphenyl)-2,6-dioxaspiro[3.4]octane-7-carboxylate

Sodium bis(trimethylsilyl)amide (1.752 mL, 1.752 mmol) was added dropwise to a solution of Example 3C (436 mg, 1.168 mmol) in tetrahydrofuran (11.7 mL) at −78° C. The reaction was stirred at −78° C. for 15 minutes, and then the cooling bath was removed to allow the reaction mixture to warm to ambient temperature. The reaction mixture was charged with saturated aqueous NH₄Cl solution and then extracted three times using ethyl acetate. The combined organic layers were washed with brine, dried over Na₂SO₄, and concentrated. The crude residue was purified by flash chromatography (ISCO CombiFlash, 0-100% ethyl acetate/heptanes) to afford the title compound (271 mg, 0.927 mmol, 79% yield). ¹H NMR (500 MHz, CDCl₃) δ ppm 7.36-7.20 (m, 1H), 7.07 (ddt, J=8.3, 2.2, 0.7 Hz, 1H), 6.75 (d, J=8.2 Hz, 1H), 4.92 (d, J=6.4 Hz, 1H), 4.64 (d, J=6.4 Hz, 1H), 4.53 (d, J=6.2 Hz, 1H), 4.45 (d, J=6.3 Hz, 1H), 4.33 (d, J=8.9 Hz, 1H), 4.27 (d, J=8.9 Hz, 1H), 3.75 (s, 3H), 3.64 (s, 3H), 3.39 (d, J=13.5 Hz, 1H), 2.30 (t, J=0.7 Hz, 3H), 2.18 (d, J=13.6 Hz, 1H). MS(ESI) m/z 293.3 (M+H)⁺.

### Example 3E

### rac-7-(2-methoxy-5-methylphenyl)-2,6-dioxaspiro[3.4]octane-7-carboxylic Acid

A mixture of KOH (520 mg, 9.27 mmol) and Example 3D (271 mg, 0.927 mmol) in 1:1:1 acetonitrile/H₂O/methanol (8 mL) was heated at 45° C. overnight. The reaction mixture was cooled to ambient temperature, diluted with water, and washed twice with tert-butyl methyl ether. The aqueous fraction was acidified using 1 M citric acid, extracted three times with ethyl acetate, washed with brine, dried over Na₂SO₄, and concentrated to afford the title compound (212 mg, 0.762 mmol, 82% yield). ¹H NMR (400 MHz, dimethyl sulfoxide-d₆) δ ppm 12.35 (s, 1H), 7.18 (d, J=2.3 Hz, 1H), 7.08-6.97 (m, 1H), 6.86 (d, J=8.2 Hz, 1H), 4.70 (d, J=6.2 Hz, 1H), 4.49 (d, J=6.1 Hz, 1H), 4.40 (d, J=6.1 Hz, 1H), 4.26 (d, J=6.1 Hz, 1H), 4.22 (d, J=8.7 Hz, 1H), 4.13 (d, J=8.7 Hz, 1H), 3.69 (s, 3H), 3.17 (d, J=13.3 Hz, 1H), 2.23 (s, 3H), 2.08 (d, J=13.4 Hz, 1H). MS(ESI) m/z 279.4 (M+H)⁺.

### Example 3F

### rac-7-(2-methoxy-5-methylphenyl)-N-(2-methylquinoline-5-sulfonyl)-2,6-dioxaspiro[3.4]octane-7-carboxamide

A mixture of 4-dimethylaminopyridine (102 mg, 0.838 mmol), 1-ethyl-3-[3-(dimethylamino)propyl]-carbodiimide hydrochloride (234 mg, 1.219 mmol), 2-methylquinoline-5-sulfonamide (169 mg, 0.762 mmol), and Example 3E (212 mg, 0.762 mmol) in dichloromethane (8 mL) was stirred at ambient temperature overnight. The mixture was acidified with 1 M citric acid, extracted three times with dichloromethane, dried over Na₂SO₄, and concentrated under a stream of nitrogen to afford the title compound (345 mg, 0.715 mmol, 94% yield). ¹H NMR (400 MHz, dimethyl sulfoxide-d₆) δ ppm 11.97 (s, 1H), 8.73 (d, J=8.9 Hz, 1H), 8.24 (dt, J=8.3, 2.1 Hz, 2H), 7.88 (dd, J=8.5, 7.4 Hz, 1H), 7.48 (d, J=8.9 Hz, 1H), 7.15 (d, J=2.2 Hz, 1H), 7.04 (dd, J=8.3, 2.2 Hz, 1H), 6.62 (d, J=8.3 Hz, 1H), 4.32 (d, J=6.1 Hz, 1H), 4.28 (d, J=6.2 Hz, 1H), 4.24 (d, J=6.2 Hz, 1H), 4.09 (d, J=8.9 Hz, 1H), 3.99 (d, J=6.2 Hz, 1H), 3.74 (d, J=8.9 Hz, 1H), 3.07 (s, 3H), 2.95 (d, J=13.3 Hz, 1H), 2.71 (s, 3H), 2.21 (s, 3H), 1.98 (d, J=13.4 Hz, 1H). MS(ESI) m/z 483.1 (M+H)⁺.

### Example 4

### (2S)-2-(2-methoxy-5-methylphenyl)-4,4-dimethyl-N-(2-methylquinoline-5-sulfonyl)oxolane-2-carboxamide

### Example 4A

### Ethyl 2-diazo-2-(2-methoxy-5-methylphenyl)acetate

The title compound was prepared according to the procedure described in Examples 3A and 3B, substituting ethyl 2-chloro-2-oxoacetate for methyl 2-chloro-2-oxoacetate. ¹H NMR (500 MHz, CDCl₃) δ ppm 7.37 (d, J=2.2 Hz, 1H), 7.04 (ddq, J=8.4, 2.2, 0.7 Hz, 1H), 6.79 (d, J=8.4 Hz, 1H), 4.30 (q, J=7.1 Hz, 2H), 3.82 (s, 3H), 2.30 (d, J=0.8 Hz, 3H), 1.32 (t, J=7.1 Hz, 3H).

### Example 4B

### ethyl 2-(3-bromo-2,2-dimethylpropoxy)-2-(2-methoxy-5-methylphenyl)acetate

Example 4A (100 mg, 0.427 mmol) was dissolved in dichloromethane (2 mL) and added dropwise over 4 hours to a mixture of 3-bromo-2,2-dimethylpropan-1-ol (0.104 mL, 0.854 mmol) and Rh₂(OAc)₄ (1.887 mg, 4.27 μmol) in dichloromethane (2 mL). The reaction was stirred overnight. The mixture was loaded onto diatomaceous earth and purified by flash chromatography (ISCO CombiFlash, 0-100% ethyl acetate/heptanes) to afford the title compound (104 mg, 0.279 mmol, 65.3% yield). ¹H NMR (500 MHz, dimethyl sulfoxide) δ ppm 7.14-7.08 (m, 2H), 6.92 (d, J=8.4 Hz, 1H), 5.11 (s, 1H), 4.13-4.07 (m, 2H), 3.74 (s, 3H), 3.46 (d, J=3.1 Hz, 2H), 3.36 (d, J=8.9 Hz, 1H), 3.24 (d, J=9.0 Hz, 1H), 2.23 (s, 3H), 1.14 (t, J=7.1 Hz, 3H), 0.98 (s, 3H), 0.95 (s, 3H).

### Example 4C

### ethyl 2-(2-methoxy-5-methylphenyl)-4,4-dimethyltetrahydrofuran-2-carboxylate

Sodium bis(trimethylsilyl)amide (1 M in tetrahydrofuran, 675 μL, 0.675 mmol) was added dropwise over 1 minute to a solution of Example 4B (180 mg, 0.482 mmol) in tetrahydrofuran (4822 μL) at −78° C. under nitrogen. The reaction was stirred at −78° C. for 15 minutes, and then the cooling bath was removed to allow reaction to return to ambient temperature. After 20 minutes, the reaction was charged with a saturated NH₄Cl solution, extracted three times with ethyl acetate, washed with brine, dried over Na₂SO₄, and concentrated under reduced pressure. The crude material was purified by flash chromatography (ISCO CombiFlash, 0-100% ethyl acetate/heptanes) to afford the title compound (104 mg, 0.356 mmol, 73.8% yield). ¹H NMR (600 MHz, CDCl₃) δ ppm 7.46 (d, J=2.3 Hz, 1H), 7.04 (ddt, J=8.2, 2.3, 0.8 Hz, 1H), 6.72 (d, J=8.1 Hz, 1H), 4.20 (dtd, J=10.7, 7.4, 6.8 Hz, 1H), 4.09-4.04 (m, 1H), 3.75-3.68 (m, 5H), 3.02 (d, J=13.3 Hz, 1H), 2.32 (d, J=0.8 Hz, 3H), 1.75 (dd, J=13.2, 0.7 Hz, 1H), 1.17 (s, 3H), 1.16-1.13 (m, 3H), 1.02 (s, 3H). MS(APCI) m/z 219.4 (M-CO₂C₂H₅)⁺.

### Example 4D

### (2S)-2-(2-methoxy-5-methylphenyl)-4,4-dimethyl-N-(2-methylquinoline-5-sulfonyl)oxolane-2-carboxamide

A mixture of KOH (192 mg, 3.42 mmol) and Example 4C (100 mg, 0.342 mmol) in 1:1:1 tetrahydrofuran/methanol/H₂O (2 mL) was heated to 45° C. overnight. The reaction mixture was cooled to ambient temperature, diluted with water, and washed twice with tert-butyl methyl ether. The aqueous layer was acidified with 1 M citric acid and then extracted three times with dichloromethane. The combined organic layers were dried over Na₂SO₄ and concentrated under reduced pressure to afford the crude residue, (2-(2-methoxy-5-methylphenyl)-4,4-dimethyltetrahydrofuran-2-carboxylic acid (80 mg, 0.303 mmol, 88% yield). MS(ESI) m/z 265.4 (M+H)⁺. The crude residue was mixed with 1-(3-dimethylaminopropyl)-3-ethylcarbodiimide hydrochloride (87 mg, 0.454 mmol), 2-methylquinoline-5-sulfonamide (67.3 mg, 0.303 mmol) and 4-(dimethylamino)pyridine (40.7 mg, 0.333 mmol) in dichloromethane (3 mL) at ambient temperature and stirred overnight. The reaction was acidified with 1 M citric acid, extracted three times with dichloromethane, dried over Na₂SO₄, and concentrated to afford the racemic title compound (136 mg, 0.290 mmol, 96% yield). The enantiomers were separated by chiral preparative supercritical fluid chromatography (Chiralpak IC column (21×250 mm, 5 micron) 13.5 mg/mL in 1:1 methanol/acetonitrile, 48 g/minutes CO₂, RT 7.2 minutes) to provide the title compound (55 mg). ¹H NMR (400 MHz, dimethyl sulfoxide-d₆) δ ppm 11.86 (s, 1H), 8.65 (d, J=8.8 Hz, 1H), 8.22 (t, J=8.5 Hz, 2H), 7.86 (t, J=7.9 Hz, 1H), 7.38 (d, J=8.9 Hz, 1H), 7.28 (d, J=2.4 Hz, 1H), 7.09-6.93 (m, 1H), 6.53 (d, J=8.3 Hz, 1H), 3.51 (d, J=8.2 Hz, 1H), 3.22 (d, J=8.3 Hz, 1H), 3.03 (s, 3H), 2.69 (s, 3H), 2.64 (d, J=13.3 Hz, 1H), 2.22 (s, 3H), 1.47 (d, J=13.3 Hz, 1H), 0.87 (s, 3H), 0.75 (s, 3H). MS(ESI) m/z 469.1 (M+H)⁺.

### Example 5

### (2R)-2-(2-methoxy-5-methylphenyl)-4,4-dimethyl-N-(2-methylquinoline-5-sulfonyl)oxolane-2-carboxamide

A mixture of KOH (192 mg, 3.42 mmol) and Example 4C (100 mg, 0.342 mmol) in 1:1:1 tetrahydrofuran/methanol/H₂O (2 mL) was heated to 45° C. overnight. The reaction mixture was cooled to ambient temperature, diluted with water, and washed twice with tert-butyl methyl ether. The aqueous layer was acidified with 1 M citric acid and then extracted three times with dichloromethane. The combined organic layers were dried over Na₂SO₄ and concentrated under reduced pressure to afford the crude residue, (2-(2-methoxy-5-methylphenyl)-4,4-dimethyltetrahydrofuran-2-carboxylic acid (80 mg, 0.303 mmol, 88% yield). MS(ESI) m/z 265.4 (M+H)⁺. The crude residue was mixed with 1-(3-dimethylaminopropyl)-3-ethylcarbodiimide hydrochloride (87 mg, 0.454 mmol), 2-methylquinoline-5-sulfonamide (67.3 mg, 0.303 mmol) and 4-(dimethylamino)pyridine (40.7 mg, 0.333 mmol) in dichloromethane (3 mL) at ambient temperature and stirred overnight. The reaction was acidified with 1 M citric acid, extracted three times with dichloromethane, dried over Na₂SO₄, and concentrated to afford the racemic title compound (136 mg, 0.290 mmol, 96% yield). The enantiomers were separated by chiral preparative supercritical fluid chromatography (Chiralpak IC column (21×250 mm, 5 micron), 13.5 mg/mL in 1:1 methanol/acetonitrile, 48 g/minute CO₂, RT 8.8 minutes) to provide the title compound (54 mg). ¹H NMR (400 MHz, dimethyl sulfoxide-d₆) δ ppm 11.86 (s, 1H), 8.65 (d, J=8.8 Hz, 1H), 8.22 (t, J=8.5 Hz, 2H), 7.86 (t, J=7.9 Hz, 1H), 7.38 (d, J=8.9 Hz, 1H), 7.28 (d, J=2.4 Hz, 1H), 7.09-6.93 (m, 1H), 6.53 (d, J=8.3 Hz, 1H), 3.51 (d, J=8.2 Hz, 1H), 3.22 (d, J=8.3 Hz, 1H), 3.03 (s, 3H), 2.69 (s, 3H), 2.64 (d, J=13.3 Hz, 1H), 2.22 (s, 3H), 1.47 (d, J=13.3 Hz, 1H), 0.87 (s, 3H), 0.75 (s, 3H). MS(ESI) m/z 469.1 (M+H)⁺.

### Example 6

### (2R)—N-(2-cyclopropylquinoline-5-sulfonyl)-2-(2-ethoxy-5-methylphenyl)oxolane-2-carboxamide

### Example 6A

### (S)-2-(2-ethoxy-5-methylphenyl)tetrahydrofuran-2-carboxylic Acid

Example 1E was separated by chiral preparative supercritical fluid chromatograph (ChiralPak IC column (30×250 mm, 5 micron), 50 mg/mL in methanol, 56 g/minutes CO₂) to afford the first-eluting peak (RT=3.4 minutes) as the title compound.

### Example 6B

### (R)—N-((2-chloroquinolin-5-yl)sulfonyl)-2-(2-ethoxy-5-methylphenyl)tetrahydrofuran-2-carboxamide

To a mixture of Example 6A (100 mg, 0.400 mmol), 3-{[(ethylimino)methylidene]amino}-N,N-dimethylpropan-1-amine-hydrogen chloride (1/1) (84 mg, 0.439 mmol), 2-chloroquinoline-5-sulfonamide (97 mg, 0.400 mmol) and N,N-dimethylpyridin-4-amine (98 mg, 0.799 mmol) was added dichloromethane (2 mL), and the reaction was stirred overnight at ambient temperature. The reaction mixture was concentrated under a stream of nitrogen. The residue was reconstituted in dimethyl sulfoxide/methanol and purified via reverse-phase HPLC (Phenomenex® Luna® C₈(2) 5 μm 100 Å AXIA column (50 mm×30 mm), gradient of acetonitrile (A) and 0.1% trifluoroacetic acid in water (B), 40 mL/minutes (0-0.5 minutes 15% A, 0.5-8.0 minutes linear gradient 15-100% A, 8.0-9.0 minutes 100% A, 9.0-9.1 minutes linear gradient 100-15% A, 9.1-10 minutes 15% A)) to afford the title compound (153 mg, 81% yield). ¹H NMR (400 MHz, dimethyl sulfoxide-d₆) δ ppm 11.90 (s, 1H), 8.79 (dd, J=9.0, 0.8 Hz, 1H), 8.32 (dd, J=7.4, 1.2 Hz, 1H), 8.24 (dt, J=8.4, 1.0 Hz, 1H), 7.97 (dd, J=8.5, 7.4 Hz, 1H), 7.56 (d, J=9.0 Hz, 1H), 7.18 (d, J=2.2 Hz, 1H), 6.96 (ddd, J=8.2, 2.3, 0.9 Hz, 1H), 6.47 (d, J=8.2 Hz, 1H), 3.88-3.78 (m, 1H), 3.65-3.47 (m, 2H), 3.02-2.90 (m, 1H), 2.65-2.54 (m, 1H), 2.20 (s, 3H), 1.75-1.52 (m, 3H), 0.68 (t, J=6.9 Hz, 3H). MS (APCI+) m/z 475.4 (M+H)⁺.

### Example 6C

### (2R)—N-(2-cyclopropylquinoline-5-sulfonyl)-2-(2-ethoxy-5-methylphenyl)oxolane-2-carboxamide

To a mixture of Example 6B (40 mg, 0.084 mmol) and (methanesulfonato-κO)[2′-(methylamino)-2-biphenylyl]palladium-dicyclohexyl(2′,6′-dimethoxy-2-biphenylyl)phosphine (1:1) (SPhos Pd G4) (3.34 mg, 4.21 μmol) in dimethyl acetamide (842 μL) was added cyclopropylzinc(II) bromide (648 μL, 0.168 mmol) in tetrahydrofuran. The reaction was heated for 2 hours at 65° C. The reaction was purified directly via reverse-phase HPLC (Phenomenex® Luna® C8(2) 5 μm 100 Å AXIA column (50 mm×30 mm), gradient of acetonitrile (A) and 0.1% trifluoroacetic acid in water (B), 40 mL/minutes (0-0.5 minutes 15% A, 0.5-8.0 minutes linear gradient 15-100% A, 8.0-9.0 minutes 100% A, 9.0-9.1 minutes linear gradient 100-15% A, 9.1-10 minutes 15% A)) to afford the title compound (39.6 mg, 98% yield). ¹H NMR (400 MHz, dimethyl sulfoxide-d₆) δ ppm 11.78 (s, 1H), 8.61 (d, J=9.0 Hz, 1H), 8.17 (dd, J=7.5, 1.2 Hz, 1H), 8.11 (d, J=8.4 Hz, 1H), 7.81 (dd, J=8.5, 7.4 Hz, 1H), 7.37 (d, J=9.0 Hz, 1H), 7.15 (d, J=2.3 Hz, 1H), 6.96 (dd, J=8.3, 2.3 Hz, 1H), 6.49 (d, J=8.2 Hz, 1H), 3.86-3.76 (m, 1H), 3.60 (d, J=8.0 Hz, 2H), 3.02-2.89 (m, 1H), 2.60-2.51 (m, 1H), 2.36-2.26 (m, 1H), 2.19 (s, 3H), 1.75-1.52 (m, 3H), 1.17-1.02 (m, 4H), 0.65 (t, J=6.9 Hz, 3H). MS (APCI+) m/z 481.4 (M+H)⁺.

### Example 7

### (2S,5S)-2-(2-methoxy-5-methylphenyl)-N-(2-methylquinoline-5-sulfonyl)-5-phenyloxolane-2-carboxamide

### Example 7A

### (S)-methyl 2-((S)-3-chloro-1-phenylpropoxy)-2-(2-methoxy-5-methylphenyl)acetate

A mixture of (S)-3-chloro-1-phenylpropan-1-ol (372 mg, 2.180 mmol) and methyl 2-diazo-2-(2-methoxy-5-methylphenyl)acetate (400 mg, 1.816 mmol; Example 3B) in dichloromethane (9.1 mL) was subjected to blue light (450 nm, 50% intensity) in a Penn M2 photoreactor for 4 hours. The reaction mixture was concentrated under reduced pressure, and the crude residue was purified by flash chromatography (ISCO CombiFlash, 0-100% ethyl acetate/heptanes) to afford the first eluting peak as the title compound (175 mg, 0.482 mmol, 53.1% yield). The absolute configuration of the ester-bearing carbon was assigned arbitrarily. ¹H NMR (600 MHz, CDCl₃) δ ppm 7.39-7.26 (m, 5H), 7.15 (d, J=2.3 Hz, 1H), 7.08-7.05 (m, 1H), 6.72 (d, J=8.4 Hz, 1H), 5.16 (s, 1H), 4.75 (dd, J=9.1, 3.9 Hz, 1H), 3.93 (ddd, J=10.7, 8.0, 6.3 Hz, 1H), 3.73 (s, 3H), 3.71-3.66 (m, 1H), 3.62 (s, 3H), 2.36 (dddd, J=14.6, 9.2, 6.3, 5.4 Hz, 1H), 2.27 (s, 3H), 2.05 (dddd, J=14.5, 8.0, 6.7, 4.0 Hz, 1H). MS(ESI) m/z 385.2 (M+Na)⁺.

### Example 7B

### (R)-methyl 2-((S)-3-chloro-1-phenylpropoxy)-2-(2-methoxy-5-methylphenyl)acetate

A mixture of (S)-3-chloro-1-phenylpropan-1-ol (372 mg, 2.180 mmol) and methyl 2-diazo-2-(2-methoxy-5-methylphenyl)acetate (400 mg, 1.816 mmol; Example 3B) in dichloromethane (9.1 mL) was subjected to blue light (450 nm, 50% intensity) in a Penn M2 photoreactor for 4 hours. The reaction mixture was concentrated under reduced pressure, and the crude residue was purified by flash chromatography (ISCO CombiFlash, 0-100% ethyl acetate/heptanes) to afford the second eluting peak as the title compound (156 mg, 0.430 mmol, 47.3% yield). The absolute configuration of the ester-bearing carbon was assigned arbitrarily. ¹H NMR (600 MHz, CDCl₃) δ ppm 7.40-7.28 (m, 5H), 7.17 (d, J=2.3 Hz, 1H), 7.12 (ddd, J=8.3, 2.3, 0.8 Hz, 1H), 6.80 (d, J=8.3 Hz, 1H), 5.12 (s, 1H), 4.43 (dd, J=9.2, 4.1 Hz, 1H), 3.74 (s, 3H), 3.64-3.58 (m, 4H), 3.49 (ddd, J=11.0, 6.1, 5.3 Hz, 1H), 2.39-2.32 (m, 1H), 2.31 (s, 3H), 1.95 (dddd, J=14.6, 8.6, 6.1, 4.2 Hz, 1H). MS(ESI) m/z 385.2 (M+Na)⁺.

### Example 7C

### (2S,5S)-2-(2-methoxy-5-methylphenyl)-N-(2-methylquinoline-5-sulfonyl)-5-phenyloxolane-2-carboxamide

Sodium bis(trimethylsilyl)amide (1 M in tetrahydrofuran, 0.965 mL, 0.965 mmol) was added to a mixture of Example 7A (175 mg, 0.482 mmol) and Example 7B (156 mg, 0.430 mmol) in tetrahydrofuran (8 mL) at 0° C. under nitrogen gas. After 2 hours, the reaction was quenched with 1 M citric acid and extracted three times with dichloromethane. The combined organic layers were dried over Na₂SO₄ and concentrated to afford a mixture of diastereomers (287 mg, 0.879 mmol). MS(APCI) m/z 517.0 (M-CO₂CH₃)⁺.

The mixture was dissolved in 1:1:1 methanol/tetrahydrofuran/water (10 mL), charged with KOH (987 mg, 17.59 mmol), and stirred overnight at 60° C. The mixture was cooled to ambient temperature, acidified with 1 M citric acid, extracted three times with dichloromethane. The combined organic layers were dried over Na₂SO₄ and concentrated under reduced pressure to provide a mixture of (2S,5S)-2-(2-methoxy-5-methylphenyl)-5-phenyltetrahydrofuran-2-carboxylic acid and (2R,5S)-2-(2-methoxy-5-methylphenyl)-5-phenyltetrahydrofuran-2-carboxylic acid (250 mg, 0.800 mmol, 91% yield). MS(ESI) m/z 313.2 (M+H)⁺.

This mixture was dissolved in dichloromethane (4 mL) and charged with 4-(dimethylamino)pyridine (108 mg, 0.880 mmol), 1-(3-dimethylaminopropyl)-3-ethylcarbodiimide hydrochloride (230 mg, 1.201 mmol), and 2-methylquinoline-5-sulfonamide (178 mg, 0.800 mmol) and then stirred at ambient temperature overnight. The mixture was acidified with 1 M citric acid and extracted three times with dichloromethane. The combined organic layers were dried over Na₂SO₄, concentrated under reduced pressure, and purified by flash chromatography (ISCO CombiFlash, 0-100% ethyl acetate/heptanes) to afford a mixture of diastereomers (242 mg).

The resulting material was separated by chiral preparative supercritical fluid chromatography (Chiralpak IC column (21×250 mm, 5 micron), 24.2 mg/mL in methanol (0.1% diethylamine), 64 g/minutes CO₂, RT 10.0 minutes) to provide the title compound. This material was further purified via reverse-phase HPLC (Waters Xbridge Prep C18 column, 42 mL/minute, 5-95% acetonitrile/0.1% trifluoroacetic acid in water) to afford the title compound (53.5 mg, 0.104 mmol, 12.9% yield) as the trifluoroacetic acid salt after lyophilization. ¹H NMR (500 MHz, CDCl₃) δ ppm 9.17 (d, J=8.9 Hz, 1H), 8.65 (dt, J=8.6, 1.1 Hz, 1H), 8.56 (dd, J=7.5, 1.0 Hz, 1H), 7.97 (dd, J=8.5, 7.5 Hz, 1H), 7.44 (d, J=9.0 Hz, 1H), 7.38-7.27 (m, 4H), 7.19-7.14 (m, 2H), 7.09 (ddd, J=8.2, 2.2, 0.8 Hz, 1H), 6.62 (d, J=8.3 Hz, 1H), 4.67 (dd, J=9.0, 6.3 Hz, 1H), 3.35 (s, 3H), 2.90 (s, 3H), 2.89-2.84 (m, 1H), 2.31 (t, J=0.7 Hz, 3H), 2.27 (tdd, J=8.1, 6.3, 4.1 Hz, 1H), 2.09 (ddd, J=13.2, 8.9, 4.2 Hz, 1H), 1.80 (dq, J=12.5, 8.9 Hz, 1H). MS(ESI) m/z 517.0 (M+H)⁺.

### Example 8

### (2R,5S)-2-(2-methoxy-5-methylphenyl)-N-(2-methylquinoline-5-sulfonyl)-5-phenyloxolane-2-carboxamide

Sodium bis(trimethylsilyl)amide (1 M in tetrahydrofuran, 0.965 mL, 0.965 mmol) was added to a mixture of Example 7A (175 mg, 0.482 mmol) and Example 7B (156 mg, 0.430 mmol) in tetrahydrofuran (8 mL) at 0° C. under nitrogen gas. After 2 hours, the reaction was quenched with 1 M citric acid and extracted three times with dichloromethane. The combined organic layers were dried over Na₂SO₄ and concentrated to afford a mixture of diastereomers (287 mg, 0.879 mmol). MS(APCI) m/z 517.0 (M-CO₂CH₃)⁺. This mixture was dissolved in 1:1:1 methanol/tetrahydrofuran/water (10 mL), charged with KOH (987 mg, 17.59 mmol), and stirred overnight at 60° C. The mixture was then cooled to ambient temperature, acidified with 1 M citric acid, and extracted three times with dichloromethane. The combined organic layers were dried over Na₂SO₄, and concentrated under reduced pressure to provide a mixture of (2S,5S)-2-(2-methoxy-5-methylphenyl)-5-phenyltetrahydrofuran-2-carboxylic acid and (2R,5S)-2-(2-methoxy-5-methylphenyl)-5-phenyltetrahydrofuran-2-carboxylic acid (250 mg, 0.800 mmol, 91% yield. MS(ESI) m/z 313.2 (M+H)⁺. The mixture was dissolved in dichloromethane (4 mL) and charged with 4-(dimethylamino)pyridine (108 mg, 0.880 mmol), 1-(3-dimethylaminopropyl)-3-ethylcarbodiimide hydrochloride (230 mg, 1.201 mmol), and 2-methylquinoline-5-sulfonamide (178 mg, 0.800 mmol) and then stirred at ambient temperature overnight. The mixture was acidified with 1 M citric acid and extracted three times with dichloromethane. The combined organic layers were dried over Na₂SO₄, concentrated under reduced pressure and purified by flash chromatography (ISCO CombiFlash, 0-100% ethyl acetate/heptanes) to afford a mixture of diastereomers (242 mg).

The resulting material was separated by chiral preparative supercritical fluid chromatography (Chiralpak IC column (21×250 mm, 5 micron) 24.2 mg/mL in methanol (0.1% diethylamine), 64 g/minute CO₂, RT 11.8 minutes) to provide the title compound. This material was further purified via reverse-phase HPLC (Waters Xbridge Prep C18 column, 42 mL/minute, 5-95% acetonitrile/0.1% trifluoroacetic acid in water) to afford the title compound (31 mg, 0.06 mmol, 7.5% yield) as the trifluoroacetic acid salt after lyophilization. ¹H NMR (600 MHz, CDCl₃) δ ppm 9.10 (d, J=8.9 Hz, 1H), 8.68 (dt, J=8.5, 1.1 Hz, 1H), 8.54 (dd, J=7.5, 1.1 Hz, 1H), 7.96 (dd, J=8.5, 7.5 Hz, 1H), 7.43 (d, J=9.0 Hz, 1H), 7.35-7.29 (m, 2H), 7.25-7.21 (m, 2H), 7.10-7.04 (m, 3H), 6.64 (d, J=8.3 Hz, 1H), 5.04 (dd, J=9.7, 5.6 Hz, 1H), 3.43 (s, 3H), 3.01-2.96 (m, 1H), 2.93 (s, 3H), 2.31 (s, 3H), 2.24 (dddd, J=12.5, 7.0, 5.6, 2.7 Hz, 1H), 2.01 (ddd, J=13.1, 10.8, 7.0 Hz, 1H), 1.89 (dddd, J=12.3, 10.8, 9.7, 7.4 Hz, 1H). MS(ESI) m/z 517.0 (M+H)⁺.

### Example 9

### (2R)-2-(2-ethoxy-5-ethylphenyl)-N-(2-methylquinoline-5-sulfonyl)oxolane-2-carboxamide

### Example 9A

### 4-chloro-1-(5-ethyl-2-hydroxyphenyl)butan-1-one

To a solution of aluminum chloride (14.69 g, 110 mmol) in dichloromethane (150 mL) was added 1-ethyl-4-methoxybenzene (10 g, 73.4 mmol) at 0° C. After 5 minutes, 4-chlorobutanoyl chloride (12.94 g, 92 mmol) was added dropwise. After 30 minutes, the reaction was warmed to 25° C. and stirred for 12 hours. The reaction was poured into 1 M aqueous HCl (1.5 L) and extracted with dichloromethane (3×1 L). The combined organic layers were washed with water, washed with brine, dried with anhydrous magnesium sulfate, filtered, and concentrated under reduced pressure. The residue was purified by column chromatography (100/1 to 20/1 petroleum ether/ethyl acetate, silica gel) to afford the title compound (10 g, 44.2 mmol, 60% yield). ¹H NMR (400 MHz, CDCl₃) δ ppm 12.06 (s, 1H), 7.58 (d, J=1.6 Hz, 1H), 7.34 (dd, J=1.9, 8.5 Hz, 1H), 6.93 (d, J=8.6 Hz, 1H), 3.70 (t, J=6.2 Hz, 2H), 3.23 (t, J=7.0 Hz, 2H), 2.63 (q, J=7.6 Hz, 2H), 2.24 (quin, J=6.6 Hz, 2H), 1.25 (t, J=7.6 Hz, 3H).

### Example 9B

### 2-(5-ethyl-2-hydroxyphenyl)tetrahydrofuran-2-carbonitrile

To a solution of Example 9A (5 g, 22.06 mmol) in methanol (50 mL) was added potassium cyanide (2.154 g, 33.1 mmol) at 15° C. The mixture was stirred at 35° C. for 48 hours, then concentrated under reduced pressure, and diluted with saturated aqueous sodium bicarbonate solution (500 mL). The aqueous phase was extracted with ethyl acetate (3×500 mL). The combined organic layers were washed with brine, dried with anhydrous magnesium sulfate, filtered, and concentrated under reduced pressure. The residue was purified by column chromatography (100/1 to 20/1 petroleum ether/ethyl acetate, silica gel) to afford the title compound (0.4 g, 1.84 mmol, 8.4% yield). ¹H NMR (400 MHz, CDCl₃) δ ppm 7.13-7.08 (m, 2H), 6.84 (d, J=8.8 Hz, 1H), 4.38-4.23 (m, 2H), 2.91-2.79 (m, 1H), 2.59 (q, J=7.7 Hz, 2H), 2.42-2.31 (m, 2H), 2.30-2.17 (m, 1H), 1.22 (t, J=7.6 Hz, 3H).

### Example 9C

### methyl 2-(5-ethyl-2-hydroxyphenyl)tetrahydrofuran-2-carboxylate

A solution of Example 9B (3.5 g, 16.11 mmol) in 12 M HCl in methanol (35 mL, 420 mmol) was stirred at 60° C. for 2 hours. The reaction mixture was concentrated, treated with water (50 mL), and then saturated aqueous sodium bicarbonate was added. The mixture was extracted with ethyl acetate (3×50 mL). The combined organic layers were washed with brine, concentrated under reduced pressure, and purified by column chromatography (100/1 to 20/1 petroleum ether/ethyl acetate, silica gel) to afford the title compound (2.23 g, 8.9 mmol, 55% yield). ¹H NMR (400 MHz, CDCl₃) δ ppm 8.43 (s, 1H), 7.06-7.00 (m, 2H), 6.81 (d, J=8.4 Hz, 1H), 4.22-4.05 (m, 2H), 3.73 (s, 3H), 2.91 (ddd, J=4.7, 7.3, 12.4 Hz, 1H), 2.57 (q, J=7.5 Hz, 2H), 2.37 (td, J=8.6, 12.6 Hz, 1H), 2.09-1.95 (m, 2H), 1.20 (t, J=7.6 Hz, 3H). MS(ESI−) m/z 249 (M−H)−.

### Example 9D

### methyl 2-(2-ethoxy-5-ethylphenyl)tetrahydrofuran-2-carboxylate

To a mixture of cesium carbonate (1.906 g, 5.85 mmol) and Example 9C (0.732 g, 2.92 mmol) was added N,N-dimethylformamide (5 mL) and iodoethane (0.705 mL, 8.77 mmol). The mixture was stirred for 16 hours at ambient temperature, and then partitioned between tert-butyl methyl ether and water. The organic layers were dried over anhydrous sodium sulfate and concentrated under reduced pressure. The crude material was purified by flash chromatography (0-50% tert-butyl methyl ether/hexanes, 24 g silica gel cartridge) to afford the title compound (812 mg). ¹H NMR (500 MHz, CDCl₃) δ ppm 7.41 (d, J=2.3 Hz, 1H), 7.09 (ddt, J=8.2, 2.4, 0.7 Hz, 1H), 6.77 (d, J=8.2 Hz, 1H), 4.17-4.10 (m, 1H), 4.09-4.01 (m, 2H), 4.00-3.92 (m, 1H), 3.68 (s, 3H), 3.14-3.06 (m, 1H), 2.64 (q, J=7.6 Hz, 2H), 2.20-2.09 (m, 1H), 1.98-1.86 (m, 2H), 1.36 (t, J=7.0 Hz, 3H), 1.25 (t, J=7.6 Hz, 3H).

### Example 9E

### (R)-methyl 2-(2-ethoxy-5-ethylphenyl)tetrahydrofuran-2-carboxylate

Example 9D (812 mg) was separated by chiral preparative supercritical fluid chromatography (Chiralpak IC column (21×250 mm, 5 micron), 81.1 mg/mL in methanol, 70 g/minutes CO₂, RT 4.1 minutes) to provide the title compound (259 mg). ¹H NMR (500 MHz, CDCl₃) δ ppm 7.38 (d, J=2.3 Hz, 1H), 7.06 (ddt, J=8.2, 2.4, 0.7 Hz, 1H), 6.74 (d, J=8.2 Hz, 1H), 4.13-4.07 (m, 1H), 4.06-3.98 (m, 2H), 3.97-3.89 (m, 1H), 3.65 (s, 3H), 3.11-3.03 (m, 1H), 2.62 (q, J=7.6 Hz, 2H), 2.17-2.07 (m, 1H), 1.95-1.83 (m, 2H), 1.33 (t, J=7.0 Hz, 3H), 1.23 (t, J=7.6 Hz, 3H).

### Example 9F

### (R)-2-(2-ethoxy-5-ethylphenyl)tetrahydrofuran-2-carboxylic Acid

A mixture of Example 9E (0.259 g, 0.931 mmol) and lithium hydroxide (0.128 g, 5.34 mmol) was combined with methanol (2 mL), tetrahydrofuran (1.5 mL), and water (2.0 mL). The reaction was stirred at 50° C. for 4 hours. The reaction was concentrated under reduced pressure and quenched by addition of 2 N aqueous citric acid (2.5 mL). The aqueous layer was extracted with dichloromethane, filtering through an aqueous/organic extraction tube. The organic layer was concentrated under a stream of nitrogen, and the residue azeotroped with toluene to afford the title compound (0.241 g, 0.912 mmol, 98% yield). ¹H NMR (600 MHz, CDCl₃) δ ppm 7.33 (d, J=2.3 Hz, 1H), 7.11 (dd, J=8.3, 2.3 Hz, 1H), 6.82 (d, J=8.3 Hz, 1H), 4.17-4.01 (m, 4H), 2.79 (ddd, J=13.4, 7.8, 6.0 Hz, 1H), 2.61 (q, J=7.6 Hz, 2H), 2.38 (dt, J=13.1, 7.6 Hz, 1H), 2.11 (dt, J=12.2, 7.3 Hz, 1H), 2.04 (dt, J=12.6, 6.3 Hz, 1H), 1.41 (t, J=7.0 Hz, 3H), 1.22 (t, J=7.6 Hz, 3H).

### Example 9G

### (2R)-2-(2-ethoxy-5-ethylphenyl)-N-(2-methylquinoline-5-sulfonyl)oxolane-2-carboxamide

To a solution of Example 9F (73 mg, 0.276 mmol) in dichloromethane (0.5 mL) was added 4-dimethylaminopyridine (40.5 mg, 0.331 mmol) and 1-(3-dimethylaminopropyl)-3-ethylcarbodiimide hydrochloride (106 mg, 0.552 mmol). The solution was stirred at ambient temperature for 20 minutes; then 2-methylquinoline-5-sulfonamide (61.4 mg, 0.276 mmol) was added, and the stirring continued for 2 hours. The reaction volume was reduced by about half under a stream of nitrogen, and the reaction quenched with 2 N aqueous citric acid (0.5 mL). The resulting precipitate was filtered, washed with water (2×0.5 mL), and triturated with methanol (2×0.5 mL) to afford the title compound (77 mg, 0.164 mmol, 59.5% yield). ¹H NMR (500 MHz, dimethyl sulfoxide-d₆) δ ppm 11.85 (s, 1H), 8.70-8.62 (m, 1H), 8.30-8.17 (m, 2H), 7.87 (dd, J=8.4, 7.4 Hz, 1H), 7.39 (d, J=8.9 Hz, 1H), 7.22 (d, J=2.3 Hz, 1H), 7.03 (dd, J=8.3, 2.3 Hz, 1H), 6.54 (d, J=8.2 Hz, 1H), 3.86 (dt, J=8.1, 6.3 Hz, 1H), 3.66 (dt, J=8.3, 6.8 Hz, 1H), 3.55 (dq, J=9.2, 6.9 Hz, 1H), 2.99 (dq, J=9.1, 6.9 Hz, 1H), 2.70 (s, 3H), 2.62-2.53 (m, 3H), 1.67 (dddd, J=24.3, 15.2, 11.1, 6.6 Hz, 3H), 1.17 (t, J=7.6 Hz, 3H), 0.70 (t, J=6.9 Hz, 3H). MS(APCI+) m/z 469 (M+H)⁺.

### Example 10

### (2S)-2-(2-ethoxy-5-ethylphenyl)-N-(2-methylquinoline-5-sulfonyl)oxolane-2-carboxamide

### Example 10A

### (S)-methyl 2-(2-ethoxy-5-ethylphenyl)tetrahydrofuran-2-carboxylate

Example 9D (812 mg) was separated by chiral preparative supercritical fluid chromatography Chiralpak IC column (21×250 mm, 5 micron), 81.1 mg/mL in methanol, 70 g/minutes CO₂, RT 4.7 minutes) to provide the title compound (259 mg). ¹H NMR (500 MHz, CDCl₃) δ ppm 7.38 (d, J=2.3 Hz, 1H), 7.06 (ddt, J=8.2, 2.4, 0.7 Hz, 1H), 6.74 (d, J=8.2 Hz, 1H), 4.13-4.07 (m, 1H), 4.06-3.98 (m, 2H), 3.97-3.89 (m, 1H), 3.65 (s, 3H), 3.11-3.03 (m, 1H), 2.62 (q, J=7.6 Hz, 2H), 2.17-2.07 (m, 1H), 1.95-1.83 (m, 2H), 1.33 (t, J=7.0 Hz, 3H), 1.23 (t, J=7.6 Hz, 3H).

### Example 10B

### (S)-2-(2-ethoxy-5-ethylphenyl)tetrahydrofuran-2-carboxylic Acid

A mixture Example 10A (190 mg, 0.683 mmol) and lithium hydroxide (128 mg, 5.34 mmol) was combined with methanol (2.0 mL), tetrahydrofuran (1.5 mL), and water (2.0 mL), and the reaction was stirred at 50° C. for 4 hours. The mixture was concentrated under reduced pressure and quenched by addition of 2 N aqueous citric acid (2.5 mL). The aqueous layer was extracted with dichloromethane, filtering through an aqueous/organic extraction tube. The organic layer was concentrated under a stream of nitrogen, and the residue was azeotroped with toluene to afford the title compound (0.179 g, 0.677 mmol, 99% yield). ¹H NMR (600 MHz, CDCl₃) δ ppm 7.34 (d, J=2.3 Hz, 1H), 7.09 (ddt, J=8.2, 2.2, 0.7 Hz, 1H), 6.80 (d, J=8.3 Hz, 1H), 4.11 (ddd, J=8.3, 7.4, 5.9 Hz, 1H), 4.08-4.01 (m, 3H), 2.84 (ddd, J=13.4, 7.8, 5.9 Hz, 1H), 2.60 (q, J=7.6 Hz, 2H), 2.26 (dt, J=13.0, 7.7 Hz, 1H), 2.10 (dp, J=12.1, 7.3 Hz, 1H), 2.00 (dddt, J=12.0, 8.0, 7.0, 5.9 Hz, 1H), 1.38 (t, J=7.0 Hz, 3H), 1.22 (t, J=7.6 Hz, 3H).

### Example 10C

### (2S)-2-(2-ethoxy-5-ethylphenyl)-N-(2-methylquinoline-5-sulfonyl)oxolane-2-carboxamide

To a solution of Example 10B (51 mg, 0.193 mmol) in dichloromethane (0.5 mL) was added 4-dimethylaminopyridine (28.3 mg, 0.232 mmol) and 1-(3-dimethylaminopropyl)-3-ethylcarbodiimide hydrochloride (74.0 mg, 0.386 mmol). The solution was stirred at ambient temperature for 20 minutes, then 2-methylquinoline-5-sulfonamide (42.9 mg, 0.193 mmol) was added, and the stirring continued for 2 hours. The reaction volume was reduced by about half under a stream of nitrogen, and the reaction quenched with 2 N aqueous citric acid (0.5 mL). The resulting precipitate was filtered, washed with water (2×0.5 mL), and triturated with methanol (2×0.5 mL) to afford the title compound (51 mg, 0.109 mmol, 56.4% yield). ¹H NMR (500 MHz, dimethyl sulfoxide-d₆) δ ppm 11.85 (s, 1H), 8.70-8.62 (m, 1H), 8.30-8.17 (m, 2H), 7.87 (dd, J=8.4, 7.4 Hz, 1H), 7.39 (d, J=8.9 Hz, 1H), 7.22 (d, J=2.3 Hz, 1H), 7.03 (dd, J=8.3, 2.3 Hz, 1H), 6.54 (d, J=8.2 Hz, 1H), 3.86 (dt, J=8.1, 6.3 Hz, 1H), 3.66 (dt, J=8.3, 6.8 Hz, 1H), 3.55 (dq, J=9.2, 6.9 Hz, 1H), 2.99 (dq, J=9.1, 6.9 Hz, 1H), 2.70 (s, 3H), 2.62-2.53 (m, 3H), 1.67 (dddd, J=24.3, 15.2, 11.1, 6.6 Hz, 3H), 1.17 (t, J=7.6 Hz, 3H), 0.70 (t, J=6.9 Hz, 3H). MS(APCI+) m/z 469 (M+H)⁺.

### Example 11

### (2S,5R)-2-(2-methoxy-5-methylphenyl)-N-(2-methylquinoline-5-sulfonyl)-5-phenyloxolane-2-carboxamide

### Example 11A

### (R)-methyl 2-((R)-3-chloro-1-phenylpropoxy)-2-(2-methoxy-5-methylphenyl)acetate

The title compound was prepared according to the procedure of Example 7A, substituting (R)-3-chloro-1-phenylpropan-1-ol for (S)-3-chloro-1-phenylpropan-1-ol. The title compound was the first eluting compound. The absolute configuration of the ester-bearing carbon was assigned arbitrarily. ¹H NMR (600 MHz, CDCl₃) δ ppm 7.39-7.26 (m, 5H), 7.15 (d, J=2.3 Hz, 1H), 7.08-7.05 (m, 1H), 6.72 (d, J=8.4 Hz, 1H), 5.16 (s, 1H), 4.75 (dd, J=9.1, 3.9 Hz, 1H), 3.93 (ddd, J=10.7, 8.0, 6.3 Hz, 1H), 3.73 (s, 3H), 3.71-3.66 (m, 1H), 3.62 (s, 3H), 2.36 (dddd, J=14.6, 9.2, 6.3, 5.4 Hz, 1H), 2.27 (s, 3H), 2.05 (dddd, J=14.5, 8.0, 6.7, 4.0 Hz, 1H). MS(ESI) m/z 385.2 (M+Na)⁺.

### Example 11B

### (S)-methyl 2-((R)-3-chloro-1-phenylpropoxy)-2-(2-methoxy-5-methylphenyl)acetate

The title compound was prepared according to the procedure of Example 7B, substituting (R)-3-chloro-1-phenylpropan-1-ol for (S)-3-chloro-1-phenylpropan-1-ol. The title compound was the second eluting compound. The absolute configuration of the ester-bearing carbon was assigned arbitrarily. ¹H NMR (600 MHz, CDCl₃) δ ppm 7.40-7.28 (m, 5H), 7.17 (d, J=2.3 Hz, 1H), 7.12 (ddd, J=8.3, 2.3, 0.8 Hz, 1H), 6.80 (d, J=8.3 Hz, 1H), 5.12 (s, 1H), 4.43 (dd, J=9.2, 4.1 Hz, 1H), 3.74 (s, 3H), 3.64-3.58 (m, 4H), 3.49 (ddd, J=11.0, 6.1, 5.3 Hz, 1H), 2.39-2.32 (m, 1H), 2.31 (s, 3H), 1.95 (dddd, J=14.6, 8.6, 6.1, 4.2 Hz, 1H). MS(ESI) m z 385.2 (M+Na)⁺.

### Example 11C

### (2S,5R)-2-(2-methoxy-5-methylphenyl)-N-(2-methylquinoline-5-sulfonyl)-5-phenyloxolane-2-carboxamide

The title compound was prepared according to the procedure of Example 7C, substituting Example 11A and Example 11B for Example 7A and Example 7B. The resulting mixture of diastereomers (420 mg) was separated by chiral preparative supercritical fluid chromatography (Chiracel OZ-H, column (21×250 mm, 5 micron), 42.0 mg/mL in 1:1 methanol/acetonitrile, 80 g/minutes CO₂, RT 17.5 minutes) to provide the title compound. ¹H NMR (600 MHz, CDCl₃) δ ppm 8.63 (dd, J=8.9, 0.8 Hz, 1H), 8.46 (d, J=7.6 Hz, 1H), 8.42-8.20 (m, 1H), 7.79 (t, J=7.7 Hz, 1H), 7.40-7.36 (m, 1H), 7.31 (dt, J=26.7, 7.7 Hz, 2H), 7.25-7.15 (m, 3H), 7.07 (d, J=8.3 Hz, 2H), 6.55 (d, J=8.3 Hz, 1H), 4.70 (s, 1H), 3.22 (s, 3H), 2.90 (dt, J=13.5, 8.1 Hz, 1H), 2.72 (s, 3H), 2.30 (d, J=3.1 Hz, 3H), 2.26 (dddd, J=12.6, 8.3, 6.6, 4.7 Hz, 1H), 2.03 (d, J=11.4 Hz, 1H), 1.79 (dq, J=12.4, 8.8 Hz, 1H). MS(ESI) m/z 517.0 (M+H)⁺.

### Example 12

### (2R,5R)-2-(2-methoxy-5-methylphenyl)-N-(2-methylquinoline-5-sulfonyl)-5-phenyloxolane-2-carboxamide

The title compound was prepared according to the procedure of Example 7C, substituting Example 11A and Example 11B for Example 7A and Example 7B. The resulting mixture of diastereomers (420 mg) was separated by chiral preparative supercritical fluid chromatography (Chiralcel OZ-H column (21×250 mm, 5 micron), 42.0 mg/mL in 1:1 methanol/acetonitrile, 80 g/minutes CO₂, RT 14.5 minutes) to provide the title compound. ¹H NMR (600 MHz, CDCl₃) δ ppm 8.59 (d, J=8.8 Hz, 1H), 8.47 (s, 1H), 8.30 (d, J=8.5 Hz, 1H), 7.79 (s, 1H), 7.34 (s, 1H), 7.26 (s, 1H), 7.25-7.17 (m, 3H), 7.11-7.01 (m, 3H), 6.59 (d, J=8.2 Hz, 1H), 5.03 (s, 1H), 3.31 (s, 3H), 3.00 (s, 1H), 2.74 (s, 3H), 2.30 (s, 3H), 2.21 (ddt, J=12.3, 5.6, 3.5 Hz, 1H), 1.99-1.84 (m, 2H). MS(ESI) m/z 517.0 (M+H)⁺.

### Example 13

### (2S)-2-{5-ethyl-2-[(2S)-2-methoxypropoxy]phenyl}-N-(2-methylquinoline-5-sulfonyl)oxolane-2-carboxamide

### Example 13A

### (S)-2-methoxypropyl 4-methylbenzenesulfonate

A mixture of (S)-2-methoxypropan-1-ol (0.5 g, 5.55 mmol), triethylamine (1.547 mL, 11.10 mmol), and 4-methylbenzene-1-sulfonyl chloride (1.587 g, 8.32 mmol) in dichloromethane (10 mL) was stirred at ambient temperature for 2 hours. The mixture was washed with a saturated solution of sodium carbonate. The organic layer was separated, dried over anhydrous MgSO₄, concentrated under reduced pressure, and purified by flash chromatography (10%-25% ethyl acetate/heptanes) to provide the title compound (1.094 g, 4.48 mmol, 81% yield). ¹H NMR (600 MHz, CDCl₃) δ ppm 7.84-7.76 (m, 2H), 7.40-7.32 (m, 2H), 3.97 (d, J=5.1 Hz, 2H), 3.56 (qt, J=6.4, 5.2 Hz, 1H), 3.31 (s, 3H), 2.46 (d, J=0.8 Hz, 3H), 1.13 (d, J=6.4 Hz, 3H). MS(ESI+) m/z 245.4 (M+H)⁺.

### Example 13B

### methyl 2-(5-ethyl-2-((S)-2-methoxypropoxy)phenyl)tetrahydrofuran-2-carboxylate

To a mixture of cesium carbonate (1.229 g, 3.77 mmol) and Example 9C (0.400 g, 1.598 mmol) was added N,N-dimethylformamide (2 mL) and Example 13A (0.421 g, 1.723 mmol). The mixture was stirred for 16 hours at ambient temperature, heated at 80° C. for 5 hours, then cooled to ambient temperature. The mixture was partitioned between tert-butyl methyl ether and water. The organic layer was concentrated and purified by flash chromatography (0-50% tert-butyl methyl ether/hexanes over 20 minutes, 24 g silica gel cartridge) to afford the title compound (92 mg, 0.285 mmol, 17.86% yield). ¹H NMR (400 MHz, CDCl₃) δ ppm 7.42 (d, J=2.3 Hz, 1H), 7.08 (dd, J=8.3, 2.3 Hz, 1H), 6.76 (d, J=8.2 Hz, 1H), 4.15-4.08 (m, 1H), 4.08-4.00 (m, 1H), 4.00-3.92 (m, 1H), 3.82-3.73 (m, 1H), 3.66 (s, 3H), 3.66-3.58 (m, 1H), 3.46-3.39 (m, 3H), 3.15-3.04 (m, 1H), 2.63 (q, J=7.6 Hz, 2H), 2.20-2.05 (m, 1H), 1.97-1.84 (m, 2H), 1.29-1.20 (m, 6H).

### Example 13C

### 2-(5-ethyl-2-((S)-2-methoxypropoxy)phenyl)tetrahydrofuran-2-carboxylic Acid

A mixture Example 13B (92 mg, 0.285 mmol) and lithium hydroxide (54.7 mg, 2.283 mmol) was combined with methanol (0.5 mL), tetrahydrofuran (0.5 mL), and water (0.5 mL), and the reaction was stirred at 50° C. for 2 hours. The reaction was concentrated under reduced pressure and quenched by addition of 2 N aqueous citric acid (1.5 mL). The aqueous layer was extracted with dichloromethane, filtering through an aqueous/organic extraction tube. The organic layer was concentrated under a stream of nitrogen to afford the title compound (75 mg, 0.243 mmol, 85% yield). ¹H NMR (600 MHz, CDCl₃) δ ppm 7.40-7.38 (m, 1H), 7.13-7.08 (m, 1H), 6.84-6.80 (m, 1H), 4.14-4.04 (m, 2H), 4.02-3.96 (m, 1H), 3.92-3.87 (m, 1H), 3.76-3.68 (m, 1H), 3.45-3.41 (m, 3H), 2.96-2.85 (m, 1H), 2.62 (q, J=7.6 Hz, 2H), 2.29-2.20 (m, 1H), 2.15-2.06 (m, 1H), 2.05-1.96 (m, 1H), 1.29-1.26 (m, 3H), 1.23 (t, J=7.6 Hz, 3H).

### Example 13D

### 2-(5-ethyl-2-((S)-2-methoxypropoxy)phenyl)-N-((2-methylquinolin-5-yl)sulfonyl)tetrahydrofuran-2-carboxamide

To a solution of Example 13C (73 mg, 0.237 mmol) in dichloromethane (0.5 mL) was added 4-dimethylaminopyridine (34.7 mg, 0.284 mmol) and 1-(3-dimethylaminopropyl)-3-ethylcarbodiimide hydrochloride (91 mg, 0.473 mmol). The solution was stirred at ambient temperature for 20 minutes, then 2-methylquinoline-5-sulfonamide (52.6 mg, 0.237 mmol) was added, and the stirring continued for 2 hours. The reaction volume was reduced by about half under a stream of nitrogen, and the reaction quenched with 2 N aqueous citric acid (0.5 mL). The resulting residue was purified by flash chromatography (0-100% ethyl acetate/hexanes, 25 g silica gel cartridge) to afford the title compound (71 mg) as a mixture of diastereomers. MS(APCI+) m/z 513 (M+H)⁺.

### Example 13E

### (2S)-2-{5-ethyl-2-[(2S)-2-methoxypropoxy]phenyl}-N-(2-methylquinoline-5-sulfonyl)oxolane-2-carboxamide

Example 13D (71 mg) was separated by chiral preparative supercritical fluid chromatography Chiralpak IC column (21×250 mm, 5 micron), 18 mg/mL in methanol, 48 g/minutes CO₂, RT 4.6 minutes) to provide a residue which was purified by flash chromatography (0-100% ethyl acetate/hexanes, 4 g silica cartridge) to afford the title compound (8.3 mg, 0.016 mmol, 6.84% yield). ¹H NMR (500 MHz, CDCl₃) δ ppm 8.71 (s, 1H), 8.51 (dd, J=8.8, 0.9 Hz, 1H), 8.42 (dd, J=7.4, 1.2 Hz, 1H), 8.25 (dt, J=8.5, 1.1 Hz, 1H), 7.77 (dd, J=8.5, 7.4 Hz, 1H), 7.27 (d, J=2.3 Hz, 1H), 7.23 (d, J=8.9 Hz, 1H), 7.04 (dd, J=8.3, 2.3 Hz, 1H), 6.50 (d, J=8.3 Hz, 1H), 4.00-3.93 (m, 1H), 3.87-3.79 (m, 1H), 3.55 (dd, J=9.3, 7.1 Hz, 1H), 3.32 (s, 3H), 3.09 (pd, J=6.4, 3.8 Hz, 1H), 3.01 (dd, J=9.3, 4.0 Hz, 1H), 2.93-2.81 (m, 1H), 2.76 (s, 3H), 2.58 (q, J=7.6 Hz, 2H), 1.93-1.77 (m, 3H), 1.21 (t, J=7.6 Hz, 3H), 0.96 (d, J=6.3 Hz, 3H). MS(APCI+) m/z 513 (M+H)⁺.

### Example 14

### (2R)-2-{5-ethyl-2-[(2S)-2-methoxypropoxy]phenyl}-N-(2-methylquinoline-5-sulfonyl)oxolane-2-carboxamide

Example 13D (71 mg) was separated by chiral preparative supercritical fluid (Chiralpak IC column (21×250 mm, 5 micron), 18 mg/mL in methanol, 48 g/minutes CO₂, RT 11.2 minutes) to provide a residue which was purified by flash chromatography (0-100% ethyl acetate/hexanes, 4 g silica gel cartridge) to afford the title compound (7.8 mg, 0.015 mmol, 6.43% yield). ¹H NMR (500 MHz, CDCl₃) δ ppm 8.71 (s, 1H), 8.51 (dd, J=8.8, 0.9 Hz, 1H), 8.42 (dd, J=7.4, 1.2 Hz, 1H), 8.25 (dt, J=8.5, 1.1 Hz, 1H), 7.77 (dd, J=8.5, 7.4 Hz, 1H), 7.27 (d, J=2.3 Hz, 1H), 7.23 (d, J=8.9 Hz, 1H), 7.04 (dd, J=8.3, 2.3 Hz, 1H), 6.50 (d, J=8.3 Hz, 1H), 4.00-3.93 (m, 1H), 3.87-3.79 (m, 1H), 3.55 (dd, J=9.3, 7.1 Hz, 1H), 3.32 (s, 3H), 3.09 (pd, J=6.4, 3.8 Hz, 1H), 3.01 (dd, J=9.3, 4.0 Hz, 1H), 2.93-2.81 (m, 1H), 2.76 (s, 3H), 2.58 (q, J=7.6 Hz, 2H), 1.93-1.77 (m, 3H), 1.21 (t, J=7.6 Hz, 3H), 0.96 (d, J=6.3 Hz, 3H). MS(APCI+) m/z 513 (M+H)⁺.

### Example 15

### (6R)-6-(2-methoxy-5-methylphenyl)-N-(2-methylquinoline-5-sulfonyl)-5-oxaspiro[2.4]heptane-6-carboxamide

### Example 15A

### methyl 2-hydroxy-2-(2-methoxy-5-methylphenyl)-5-(trimethylsilyl)pent-4-ynoate

To a mixture of (R)-(+)-2,2′-bis(diphenylphosphino)-1,1′-binaphthalene (120 mg, 0.192 mmol) and copper(II) iso-butyrate (28.5 mg, 0.120 mmol) which was purged with nitrogen was added tetrahydrofuran (6.0 mL), and the resulting solution was agitated at ambient temperature for 30 minutes. Lithium tert-butoxide (0.192 mL, 0.192 mmol, 1 M in tetrahydrofuran) was added and the solution was agitated at ambient temperature for 10 minutes. The solution was cooled to below −60° C., and then trimethyl(3-(4,4,5,5-tetramethyl-1,3,2-dioxaborolan-2-yl)prop-1-yn-1-yl)silane (858 mg, 3.60 mmol) [CAS #129217-85-2] was added followed by addition of methyl 2-(2-methoxy-5-methylphenyl)-2-oxoacetate (500 mg, 2.401 mmol) [CAS #1267006-90-5]. The temperature was maintained between −65° and −58° C. for 5 hours. The reaction was quenched with a solution of diethanolamine (0.445 mL, 4.64 mmol) in ethyl acetate (20 mL), and the solution stirred for 15 minutes. Water (20 mL) was added, and the biphasic mixture stirred for 15 minutes. The organic layers were separated, washed with water, washed with brine, and dried over anhydrous sodium sulfate. The organic layers were concentrated under reduced pressure, and the crude material purified by chromatography (0-50% tert-butyl methyl ether in 1:1 dichloromethane/heptanes over 25 minutes, 40 mL/min, Grace Reveleris 40 g column) to afford the title compound (656 mg, 2.047 mmol, 85% yield). ¹H NMR (400 MHz, dimethyl sulfoxide-d₆) δ ppm 7.32 (dd, J=2.3, 0.7 Hz, 1H), 7.02 (ddd, J=8.2, 2.3, 0.8 Hz, 1H), 6.80 (d, J=8.2 Hz, 1H), 6.11 (s, 1H), 3.61 (s, 3H), 3.53 (s, 3H), 2.84 (q, J=17.1 Hz, 2H), 2.23 (s, 3H), −0.05 (s, 9H).

### Example 15B

### methyl 2-hydroxy-2-(2-methoxy-5-methylphenyl)pent-4-ynoate

Potassium carbonate (1.567 g, 11.34 mmol) was added to a solution of Example 15A (1.817 g, 5.67 mmol) in anhydrous methanol (24 mL), then the mixture was agitated for 1 hour at ambient temperature. The reaction was diluted with tert-butyl methyl ether (100 mL), washed with water, washed with vbrine, and dried over anhydrous sodium sulfate. The organic layer was concentrated under reduced pressure to afford the title compound (1.430 g, 5.76 mmol, 102% yield). ¹H NMR (600 MHz, dimethyl sulfoxide-d₆) δ ppm 7.35 (dd, J=2.3, 0.8 Hz, 1H), 7.05 (ddd, J=8.2, 2.3, 0.8 Hz, 1H), 6.84 (d, J=8.2 Hz, 1H), 6.18 (s, 1H), 3.65 (s, 3H), 3.56 (s, 3H), 2.93 (dd, J=16.9, 2.7 Hz, 1H), 2.85 (dd, J=16.9, 2.7 Hz, 1H), 2.55 (t, J=2.7 Hz, 1H), 2.26 (s, 3H).

### Example 15C

### methyl 2-(2-methoxy-5-methylphenyl)-4-oxotetrahydrofuran-2-carboxylate

To a solution of Example 15B (1.430 g, 5.76 mmol) in 1,2-dichloroethane (20 mL) at 0° C. was added 3,5-dichloropyridine N-oxide (1.889 g, 11.52 mmol), methanesulfonic acid (0.45 mL, 6.94 mmol) and [bis(trifluoromethanesulfonyl)imidate](triphenylphosphine)gold(I) (2:1) toluene adduct (0.136 g, 0.086 mmol). The solution was stirred at 0° C. for 3 hours, and then for 16 hours at ambient temperature. The reaction was diluted with dichloromethane (100 mL), and the organic layers were washed with saturated aqueous sodium bicarbonate (2×100 mL). The organic layers were dried over anhydrous sodium sulfate, filtered, and concentrated under reduced pressure. The residue was purified by chromatography (0-100% tert-butyl methyl ether in 1:1 dichloromethane/heptanes over 35 minutes, 60 mL/minute, Grace Reveleris 80 g column) to afford the title compound (0.5996 g, 2.269 mmol, 39.4% yield). ¹H NMR (500 MHz, dimethyl sulfoxide-d₆) δ ppm 7.38 (dd, J=2.3, 0.8 Hz, 1H), 7.14 (ddd, J=8.2, 2.3, 0.8 Hz, 1H), 6.95 (d, J=8.2 Hz, 1H), 4.33 (dt, J=16.6, 1.1 Hz, 1H), 4.07 (d, J=16.6 Hz, 1H), 3.71 (s, 3H), 3.62 (s, 3H), 3.28 (dt, J=17.9, 0.7 Hz, 1H), 2.57 (dd, J=18.0, 1.2 Hz, 1H), 2.30 (s, 3H). MS(APCI+) m/z 265 (M+H)⁺.

### Example 15D

### methyl 2-(2-methoxy-5-methylphenyl)-4-methylenetetrahydrofuran-2-carboxylate

A solution of potassium hexamethyldisilazide (1.760 mL, 0.880 mmol, 0.5 M in toluene) was added dropwise to a suspension of methyltriphenylphosphonium bromide (314 mg, 0.880 mmol) in anhydrous toluene (3 mL) at ambient temperature and the resulting solution was stirred for 30 minutes. A solution of Example 15C (155 mg, 0.587 mmol) in tetrahydrofuran (1 mL) was added dropwise at ambient temperature, and the mixture was stirred for 16 hours at ambient temperature. The reaction was quenched with saturated aqueous ammonium chloride (15 mL), and the aqueous layer was extracted with ethyl acetate (3×30 mL). The combined organic layers were dried over anhydrous sodium sulfate, filtered, and concentrated under reduced pressure. The residue was purified by chromatography (0-50% tert-butyl methyl ether in heptanes, silica gel) to provide the title compound (0.103 g, 0.393 mmol, 67.0% yield). ¹H NMR (600 MHz, CDCl₃) δ ppm 7.40 (dd, J=2.3, 0.8 Hz, 1H), 7.09 (ddq, J=8.3, 2.3, 0.8 Hz, 1H), 6.78 (d, J=8.2 Hz, 1H), 5.02 (p, J=2.2 Hz, 1H), 4.95 (p, J=2.2 Hz, 1H), 4.61 (dqd, J=12.7, 1.9, 0.8 Hz, 1H), 4.56 (dqd, J=12.7, 2.0, 1.1 Hz, 1H), 3.77 (s, 3H), 3.72-3.67 (m, 4H), 2.66 (dddd, J=15.6, 3.6, 2.4, 1.2 Hz, 1H), 2.33 (d, J=0.8 Hz, 3H). MS(APCI+) m/z 263 (M+H)⁺.

### Example 15E

### methyl 6-(2-methoxy-5-methylphenyl)-5-oxaspiro[2.4]heptane-6-carboxylate

To a solution of Example 15D (156.5 mg, 0.597 mmol) and chloroiodomethane (0.16 mL, 2.204 mmol) in 1,2-dichloroethane (3.0 mL) at 0° C. was added diethylzinc (1.10 mL, 1.10 mmol, 1.0 M in hexanes). After stirring at 0° C. for 2 hours, the reaction was allowed to warm to ambient temperature and stirred for 2.5 hours. The reaction was quenched with saturated aqueous ammonium chloride (10 mL) and extracted with dichloromethane (2×30 mL). The combined organic extracts were dried over anhydrous sodium sulfate and concentrated under reduced pressure. The residue was purified by flash chromatography (0-100% tert-butyl methyl ether in heptanes over 35 minutes, 40 mL/minutes, 40 g silica gel cartridge) to afford the title compound (37.7 mg, 0.136 mmol, 22.87% yield). MS(APCI+) m/z 277 (M+H)⁺.

### Example 15F

### 6-(2-methoxy-5-methylphenyl)-5-oxaspiro[2.4]heptane-6-carboxylic Acid

To a solution of Example 15E (37.7 mg, 0.136 mmol) in tetrahydrofuran (1 mL) and methanol (1 mL) was added 3 N aqueous sodium hydroxide (0.25 mL, 0.750 mmol). The solution was heated at 40° C. for 2 hours and then stirred for 16 hours at ambient temperature. The reaction was acidified with 1.0 M aqueous citric acid (1 mL), extracted with dichloromethane (3×4 mL) on a 6 mL Isolute phase separator. The organic layers were concentrated to afford the title compound (37.8 mg, 0.144 mmol, 106% yield). MS(APCI+) m/z 263 (M+H)⁺.

### Example 15G

### 6-(2-methoxy-5-methylphenyl)-N-((2-methylquinolin-5-yl)sulfonyl)-5-oxaspiro[2.4]heptane-6-carboxamide

To a solution of Example 15F (37.8 mg, 0.144 mmol), 1-(3-dimethylaminopropyl)-3-ethylcarbodiimide hydrochloride (55.3 mg, 0.288 mmol), and 4-dimethylaminopyridine (22.01 mg, 0.180 mmol) in anhydrous dichloromethane (2.0 mL) was added 2-methylquinoline-5-sulfonamide (32.0 mg, 0.144 mmol). The mixture was heated for 2 hours at 38° C., then quenched with 1.0 M aqueous citric acid (1.5 mL), and extracted with dichloromethane (3×4 mL) on a 6 mL Isolute phase separator. The crude material was purified by flash chromatography (0-100% tert-butyl methyl ether in dichloromethane over 25 minutes, 35 mL/minute, followed by 0-25% methanol in dichloromethane over 15 minutes, 35 mL/minutes, RediSep® Rf Gold 24 g cartridge) to afford the title compound (50.9 mg, 0.109 mmol, 76% yield). ¹H NMR (500 MHz, dimethyl sulfoxide-d₆) δ ppm 11.96 (s, 1H), 8.84 (dd, J=8.8, 0.8 Hz, 1H), 8.27 (d, J=7.2 Hz, 1H), 8.24 (d, J=8.4 Hz, 1H), 7.89 (t, J=7.9 Hz, 1H), 7.49 (d, J=8.9 Hz, 1H), 7.24 (d, J=2.3 Hz, 1H), 7.03 (dd, J=8.5, 2.2 Hz, 1H), 6.63 (d, J=8.3 Hz, 1H), 3.75 (d, J=7.9 Hz, 1H), 3.52 (d, J=7.9 Hz, 1H), 3.12 (s, 3H), 2.71 (s, 3H), 2.48 (d, J=12.7 Hz, 1H), 2.21 (s, 3H), 1.96 (d, J=12.9 Hz, 1H), 0.50-0.39 (m, 2H), 0.26-0.18 (m, 1H), 0.16-0.08 (m, 1H). MS(APCI+) m/z 467 (M+H)⁺. ˜68.8% ee by ChiralPak IC.

### Example 15H

### (6R)-6-(2-methoxy-5-methylphenyl)-N-(2-methylquinoline-5-sulfonyl)-5-oxaspiro[2.4]heptane-6-carboxamide

The enantiomers of Example 15G (50.9 mg) were separated by chiral preparative supercritical fluid chromatography (Chiralpak IC column (21×250 mm, 5 micron), 5.09 mg/mL in methanol, 70 g/minutes CO₂, RT 11.8 minutes) to provide the title compound (35.2 mg, 0.075 mmol, 69.2% yield). ¹H NMR (500 MHz, dimethyl sulfoxide-d₆) δ ppm 11.96 (s, 1H), 8.84 (dd, J=8.8, 0.8 Hz, 1H), 8.27 (d, J=7.2 Hz, 1H), 8.24 (d, J=8.4 Hz, 1H), 7.89 (t, J=7.9 Hz, 1H), 7.49 (d, J=8.9 Hz, 1H), 7.24 (d, J=2.3 Hz, 1H), 7.03 (dd, J=8.5, 2.2 Hz, 1H), 6.63 (d, J=8.3 Hz, 1H), 3.75 (d, J=7.9 Hz, 1H), 3.52 (d, J=7.9 Hz, 1H), 3.12 (s, 3H), 2.71 (s, 3H), 2.48 (d, J=12.7 Hz, 1H), 2.21 (s, 3H), 1.96 (d, J=12.9 Hz, 1H), 0.50-0.39 (m, 2H), 0.26-0.18 (m, 1H), 0.16-0.08 (m, 1H). MS(APCI+) m/z 467 (M+H)⁺.

### Example 16

### (6S)-6-(2-methoxy-5-methylphenyl)-N-(2-methylquinoline-5-sulfonyl)-5-oxaspiro[2.4]heptane-6-carboxamide

The enantiomers of Example 15G (50.9 mg) were separated by chiral preparative supercritical fluid chromatography (Chiralpak IC column (21×250 mm, 5 micron), 5.09 mg/mL in methanol, 70 g/minutes CO₂, RT 4.6 minutes) to provide the title compound (4.9 mg, 10.50 μmol, 9.63% yield). ¹H NMR (500 MHz, dimethyl sulfoxide-d₆) δ ppm 11.96 (s, 1H), 8.84 (dd, J=8.8, 0.8 Hz, 1H), 8.27 (d, J=7.2 Hz, 1H), 8.24 (d, J=8.4 Hz, 1H), 7.89 (t, J=7.9 Hz, 1H), 7.49 (d, J=8.9 Hz, 1H), 7.24 (d, J=2.3 Hz, 1H), 7.03 (dd, J=8.5, 2.2 Hz, 1H), 6.63 (d, J=8.3 Hz, 1H), 3.75 (d, J=7.9 Hz, 1H), 3.52 (d, J=7.9 Hz, 1H), 3.12 (s, 3H), 2.71 (s, 3H), 2.48 (d, J=12.7 Hz, 1H), 2.21 (s, 3H), 1.96 (d, J=12.9 Hz, 1H), 0.50-0.39 (m, 2H), 0.26-0.18 (m, 1H), 0.16-0.08 (m, 1H). MS(APCI+) m/z 467 (M+H)⁺.

### Example 17

### 2-(5-ethyl-2-phenoxyphenyl)-N-(2-methylquinoline-5-sulfonyl)oxolane-2-carboxamide

### Example 17A

### 2-(5-ethyl-2-phenoxyphenyl)tetrahydrofuran-2-carboxylic Acid

A suspension of iodobenzene (0.094 mL, 0.827 mmol), potassium phosphate (351 mg, 1.654 mmol), Example 9C (138 mg, 0.551 mmol), and copper(I) iodide (10.50 mg, 0.055 mmol) in N,N-dimethylformamide (1 mL) was sparged with nitrogen for 5 minutes, and then stirred at 110° C. for 20 hours. The reaction was quenched by careful addition of 2 N aqueous citric acid (1 mL). The crude reaction mixture was quenched with water and then passed through an aqueous/organic separator tube with dichloromethane (3×4 mL). The organic layers were concentrated, and the crude material purified by reverse-phase preparative HPLC (Phenomenex® Luna® C8(2) 5 μm 100 Å AXIA™ column (30 mm×150 mm), gradient of acetonitrile (A) and 0.1% trifluoroacetic acid in water (B), 50 mL/minute (0-0.5 minutes 10% A, 0.5-7.0 minutes linear gradient 10-95% A, 7.0-10.0 minutes 95% A, 10.0-12.0 minutes linear gradient 95-10% A)) to afford the title compound (67 mg, 0.214 mmol, 38.9% yield). ¹H NMR (600 MHz, CDCl₃) δ ppm 7.44 (d, J=2.3 Hz, 1H), 7.31-7.26 (m, 2H), 7.09-7.04 (m, 2H), 6.99-6.94 (m, 2H), 6.77 (d, J=8.2 Hz, 1H), 4.11-4.03 (m, 2H), 2.84 (ddd, J=13.1, 7.7, 5.4 Hz, 1H), 2.65 (q, J=7.6 Hz, 2H), 2.28 (dt, J=13.1, 7.9 Hz, 1H), 2.09 (dp, J=12.2, 7.6 Hz, 1H), 2.02 (dddt, J=12.3, 8.0, 6.9, 5.5 Hz, 1H), 1.24 (t, J=7.6 Hz, 3H). MS(APCI+) m/z 313 (M+H)⁺.

### Example 17B

### 2-(5-ethyl-2-phenoxyphenyl)-N-(2-methylquinoline-5-sulfonyl)oxolane-2-carboxamide

To a solution of Example 17A (66 mg, 0.211 mmol) in dichloromethane (1 mL) was added 4-dimethylaminopyridine (40 mg, 0.327 mmol) and 1-(3-dimethylaminopropyl)-3-ethylcarbodiimide hydrochloride (78 mg, 0.407 mmol). The solution was stirred at ambient temperature for 20 minutes, then 2-methylquinoline-5-sulfonamide (47.0 mg, 0.211 mmol) was added, and the stirring continued for 2 hours. The reaction volume was reduced by about half under a stream of nitrogen, and the reaction was quenched with 2 N aqueous citric acid (0.5 mL). The aqueous layer was removed via pipette, and the resulting residue was purified by flash chromatography (0-100% (3:1 ethyl acetate/ethanol)/hexanes, 12 g silica gel cartridge) to afford the title compound (95 mg). ¹H NMR (600 MHz, CDCl₃) δ ppm 9.10 (s, 1H), 8.71 (dd, J=8.8, 0.9 Hz, 1H), 8.43 (dd, J=7.5, 1.2 Hz, 1H), 8.25 (dt, J=8.4, 1.1 Hz, 1H), 7.74 (dd, J=8.4, 7.4 Hz, 1H), 7.26 (d, J=3.4 Hz, 1H), 7.20 (d, J=2.2 Hz, 1H), 7.16-7.08 (m, 2H), 7.03-6.95 (m, 2H), 6.55-6.45 (m, 3H), 3.98-3.89 (m, 2H), 2.72 (s, 3H), 2.63 (ddd, J=13.4, 8.0, 5.6 Hz, 1H), 2.54 (q, J=7.6 Hz, 2H), 2.21 (dt, J=13.2, 7.8 Hz, 1H), 2.00-1.82 (m, 2H), 1.18 (t, J=7.6 Hz, 3H). MS(APCI+) m/z 517 (M+H)⁺.

### Example 18

### 2-{5-ethyl-2-[(2-methoxypyridin-3-yl)oxy]phenyl}-N-(2-methylquinoline-5-sulfonyl)oxolane-2-carboxamide

### Example 18A

### 2-(5-ethyl-2-((2-methoxypyridin-3-yl)oxy)phenyl)tetrahydrofuran-2-carboxylic acid

A suspension of 3-iodo-2-methoxypyridine (0.132 mL, 0.995 mmol), potassium phosphate (422 mg, 1.99 mmol), Example 9C (166 mg, 0.663 mmol), and copper(I) iodide (12.63 mg, 0.066 mmol) in N,N-dimethylformamide (1 mL) was sparged with nitrogen for 5 minutes, and then stirred at 110° C. for 20 hours. The reaction was quenched by careful addition of 2 N aqueous citric acid (1 mL). The crude reaction mixture was quenched with water and then passed through an aqueous/organic separator tube with dichloromethane (3×4 mL). The organic layers were concentrated, and the crude material purified by reverse-phase preparative HPLC (Phenomenex® Luna® C8(2) 5 μm 100 Å AXIA™ column (30 mm×150 mm), gradient of acetonitrile (A) and 0.1% trifluoroacetic acid in water (B), 50 mL/minute (0-0.5 minutes 10% A, 0.5-7.0 minutes linear gradient 10-95% A, 7.0-10.0 minutes 95% A, 10.0-12.0 minutes linear gradient 95-10% A)) to provide the title compound (28 mg, 0.082 mmol, 12.29% yield). ¹H NMR (500 MHz, CDCl₃) δ ppm 7.91 (dd, J=5.0, 1.6 Hz, 1H), 7.43 (d, J=2.2 Hz, 1H), 7.15 (dd, J=7.7, 1.6 Hz, 1H), 7.11-7.03 (m, 1H), 6.82 (dd, J=7.7, 5.0 Hz, 1H), 6.66 (d, J=8.3 Hz, 1H), 4.14-4.07 (m, 1H), 4.03 (q, J=7.5 Hz, 1H), 3.97 (s, 3H), 2.91 (ddd, J=13.0, 7.6, 5.3 Hz, 1H), 2.64 (q, J=7.6 Hz, 2H), 2.41 (dt, J=13.0, 8.0 Hz, 1H), 2.15-1.98 (m, 2H), 1.24 (t, J=7.6 Hz, 3H). MS(APCI+) m/z 344 (M+H)⁺.

### Example 18B

### 2-{5-ethyl-2-[(2-methoxypyridin-3-yl)oxy]phenyl}-N-(2-methylquinoline-5-sulfonyl)oxolane-2-carboxamide

To a solution of Example 18A (28 mg, 0.082 mmol) in dichloromethane (1 mL) was added 4-dimethylaminopyridine (19.9 mg, 0.163 mmol) and 1-(3-dimethylaminopropyl)-3-ethylcarbodiimide hydrochloride (31.3 mg, 0.163 mmol). The solution was stirred at ambient temperature for 20 minutes, then 2-methylquinoline-5-sulfonamide (18.12 mg, 0.082 mmol) was added, and the stirring continued for 2 hours. The reaction volume was reduced by about half under a stream of nitrogen, and the reaction quenched with 2 N aqueous citric acid (0.5 mL). The resulting residue was purified by chromatography (0-100% ethyl acetate/hexanes, 12 g silica gel cartridge) to afford the title compound (38 mg). ¹H NMR (500 MHz, CDCl₃) δ ppm 9.83 (s, 1H), 8.87 (dd, J=8.9, 0.8 Hz, 1H), 8.40 (dd, J=7.4, 1.2 Hz, 1H), 8.21 (dt, J=8.5, 1.1 Hz, 1H), 7.87 (dd, J=4.5, 2.1 Hz, 1H), 7.71 (dd, J=8.5, 7.5 Hz, 1H), 7.29-7.25 (m, 1H), 7.11 (d, J=2.2 Hz, 1H), 6.97 (dd, J=8.3, 2.2 Hz, 1H), 6.76-6.65 (m, 2H), 6.51 (d, J=8.3 Hz, 1H), 4.04 (s, 3H), 3.88 (td, J=8.1, 5.1 Hz, 1H), 3.79 (q, J=7.6 Hz, 1H), 2.82-2.74 (m, 1H), 2.72 (s, 3H), 2.51 (q, J=7.6 Hz, 2H), 2.24 (dt, J=12.9, 8.4 Hz, 1H), 1.96-1.86 (m, 1H), 1.86-1.77 (m, 1H), 1.14 (t, J=7.6 Hz, 3H). MS(APCI+) m/z 483 (M+H)⁺.

### Example 19

### (2R,4R)-2-(2-methoxy-5-methylphenyl)-4-methyl-N-(2-methylquinoline-5-sulfonyl)oxolane-2-carboxamide

### Example 19A

### 2-(2-methoxy-5-methylphenyl)-4-methylenetetrahydrofuran-2-carboxylic Acid

Example 15D (160.7 mg, 0.613 mmol) was dissolved in tetrahydrofuran (2.0 mL) and methanol (2.0 mL), and then 3 N aqueous sodium hydroxide (1.0 mL, 3.00 mmol) was added. The solution was heated at 40° C. for 2 hours and then stirred for 16 hours at ambient temperature. The reaction was quenched with 1.0 M aqueous citric acid (3 mL), extracted with dichloromethane (3×10 mL) on a 25 mL Isolute phase separator, and the organic layer was concentrated under reduced pressure to afford the title compound (155.5 mg, 0.626 mmol, 102% yield).

### Example 19B

### 2-(2-methoxy-5-methylphenyl)-4-methylene-N-((2-methylquinolin-5-yl)sulfonyl)tetrahydrofuran-2-carboxamide

To a solution of Example 19A (155.5 mg, 0.626 mmol), 1-(3-dimethylaminopropyl)-3-ethylcarbodiimide hydrochloride (240 mg, 1.253 mmol), and 4-dimethylaminopyridine (96 mg, 0.783 mmol) in anhydrous dichloromethane (5.0 mL) was added 2-methylquinoline-5-sulfonamide (139 mg, 0.626 mmol). The reaction was heated for 2 hours at 38° C., then quenched with 1.0 M aqueous citric acid (4 mL), and extracted with dichloromethane (2×5 mL) on a 25 mL Isolute phase separator. The organic layers were concentrated under reduced pressure, and the crude material purified by flash chromatography (0-100% tert-butyl methyl ether in dichloromethane over 25 minutes, 40 mL/minute, followed by 0-25% methanol in dichloromethane over 15 minutes, 40 mL/minute, RediSep® Rf Gold 40 g column) to afford the title compound (220.3 mg, 0.487 mmol, 78% yield). ¹H NMR (500 MHz, dimethyl sulfoxide-d₆) δ ppm 12.06 (s, 1H), 8.76 (dd, J=8.9, 0.8 Hz, 1H), 8.28-8.21 (m, 2H), 7.89 (dd, J=8.4, 7.4 Hz, 1H), 7.48 (d, J=8.9 Hz, 1H), 7.22 (d, J=2.2 Hz, 1H), 7.05 (ddd, J=8.2, 2.3, 0.9 Hz, 1H), 6.62 (d, J=8.3 Hz, 1H), 4.86 (p, J=2.3 Hz, 1H), 4.81 (p, J=2.2 Hz, 1H), 4.36 (dq, J=13.4, 2.1 Hz, 1H), 4.09 (dp, J=13.3, 2.1 Hz, 1H), 3.23 (dt, J=16.2, 1.7 Hz, 1H), 3.06 (s, 3H), 2.72 (s, 3H), 2.45 (dq, J=16.5, 2.2 Hz, 1H), 2.23 (s, 3H). MS(APCI+) m/z 453 (M+H)⁺.

### Example 19C

### (R)-2-(2-methoxy-5-methylphenyl)-4-methylene-N-((2-methylquinolin-5-yl)sulfonyl)tetrahydrofuran-2-carboxamide

The enantiomers of Example 19B (220 mg) were separated by chiral preparative supercritical fluid chromatography (Chiralpak IC column (21×250 mm, 5 micron), 11 mg/mL in methanol, 70 g/minutes CO₂, RT 3.4 minutes) to provide the title compound (190.0 mg, 0.420 mmol, 86% yield). ¹H NMR (500 MHz, dimethyl sulfoxide-d₆) δ ppm 12.06 (s, 1H), 8.76 (dd, J=8.8, 0.8 Hz, 1H), 8.28-8.21 (m, 2H), 7.89 (dd, J=8.4, 7.4 Hz, 1H), 7.48 (d, J=8.9 Hz, 1H), 7.22 (d, J=2.3 Hz, 1H), 7.05 (ddd, J=8.2, 2.3, 0.8 Hz, 1H), 6.62 (d, J=8.3 Hz, 1H), 4.86 (p, J=2.2 Hz, 1H), 4.81 (p, J=2.2 Hz, 1H), 4.36 (dd, J=13.3, 2.1 Hz, 1H), 4.09 (dt, J=12.7, 1.9 Hz, 1H), 3.23 (dt, J=16.0, 1.7 Hz, 1H), 3.06 (s, 3H), 2.72 (s, 3H), 2.45 (dq, J=16.4, 2.2 Hz, 1H), 2.23 (s, 3H).

### Example 19D

### (2R)-2-(2-methoxy-5-methylphenyl)-4-methyl-N-((2-methylquinolin-5-yl)sulfonyl)tetrahydrofuran-2-carboxamide

Example 19C (180.6 mg, 0.399 mmol) and tetrahydrofuran (1 mL) were degassed in a 20 mL Barnstead reactor with a glass liner under inert atmosphere containing Wilkinson's catalyst (13.29 mg, 0.014 mmol). The vessel was degassed several times with inert gas followed by hydrogen gas and stirred for 20.9 hours at 50 psi and 35° C. The reaction was filtered and concentrated to afford the title compound (189.9 mg, 0.418 mmol, 105% yield). MS(APCI+) m/z 455 (M+H)⁺.

### Example 19E

### (2R,4R)-2-(2-methoxy-5-methylphenyl)-4-methyl-N-(2-methylquinoline-5-sulfonyl)oxolane-2-carboxamide

The enantiomers of Example 19D (188.9 mg) were separated by chiral preparative supercritical fluid chromatography (Chiralcel OZ-H column (21×250 mm, 5 micron 19 mg/mL in methanol, 80 g/minutes CO₂, RT 8.0 minutes) to provide the title compound (47.2 mg, 0.104 mmol, 24.86% yield). ¹H NMR (600 MHz, dimethyl sulfoxide-d₆) δ ppm 11.87 (s, 1H), 8.74 (d, J=8.9 Hz, 1H), 8.23 (t, J=8.7 Hz, 2H), 7.87 (t, J=7.9 Hz, 1H), 7.45 (d, J=8.9 Hz, 1H), 7.22 (d, J=2.2 Hz, 1H), 7.01 (dd, J=8.2, 2.2 Hz, 1H), 6.57 (d, J=8.2 Hz, 1H), 3.82 (t, J=7.5 Hz, 1H), 3.32 (t, J=8.2 Hz, 1H), 3.03 (s, 3H), 2.79 (dd, J=12.8, 7.1 Hz, 1H), 2.70 (s, 3H), 2.21 (s, 3H), 2.08-2.00 (m, 1H), 1.30 (dd, J=12.9, 9.7 Hz, 1H), 0.84 (d, J=6.5 Hz, 3H). MS(APCI+) m/z 455 (M+H)⁺.

### Example 20

### (2R,4S)-2-(2-methoxy-5-methylphenyl)-4-methyl-N-(2-methylquinoline-5-sulfonyl)oxolane-2-carboxamide

The enantiomers of Example 19D (188.9 mg) were separated by chiral preparative supercritical fluid chromatography (Chiralcel OZ-H column (21×250 mm, 5 micron 19 mg/mL in methanol, 80 g/minutes CO₂, RT 9.5 minutes) to provide the title compound (91.5 mg, 0.201 mmol, 48.2% yield). ¹H NMR (600 MHz, dimethyl sulfoxide) δ ppm 11.88 (s, 1H), 8.80 (d, J=8.9 Hz, 1H), 8.25 (d, J=7.4 Hz, 1H), 8.22 (d, J=8.4 Hz, 1H), 7.87 (t, J=7.9 Hz, 1H), 7.49 (d, J=8.9 Hz, 1H), 7.15 (d, J=2.2 Hz, 1H), 7.01 (dd, J=8.3, 2.2 Hz, 1H), 6.59 (d, J=8.2 Hz, 1H), 3.98 (t, J=7.6 Hz, 1H), 3.00 (s, 3H), 2.70 (s, 3H), 2.22 (s, 3H), 2.16-2.08 (m, 1H), 2.06 (dd, J=12.7, 9.4 Hz, 1H), 1.98 (dd, J=12.7, 7.4 Hz, 1H), 0.81 (d, J=6.4 Hz, 3H). MS(APCI+) m/z 455 (M+H)⁺.

### Example 21

### 2-(2-{[2-(difluoromethoxy)pyridin-3-yl]oxy}-5-ethylphenyl)-N-(2-methylquinoline-5-sulfonyl)oxolane-2-carboxamide

### Example 21A

### 2-(2-((2-(difluoromethoxy)pyridin-3-yl)oxy)-5-ethylphenyl)tetrahydrofuran-2-carboxylic Acid

A suspension of 3-bromo-2-(difluoromethoxy)pyridine (161 mg, 0.719 mmol), potassium phosphate (382 mg, 1.798 mmol), Example 9C (150 mg, 0.599 mmol), and copper(I) iodide (11.41 mg, 0.060 mmol) in N,N-dimethylformamide (1 mL) was sparged with nitrogen for 5 minutes, and then stirred at 110° C. for 20 hours. The reaction was quenched by careful addition of 2 N aqueous citric acid (1 mL), diluted with more citric acid, and extracted with tert-butyl methyl ether. The combined organic layers were concentrated under reduced pressure, and the crude material purified by flash chromatography (0-100% ethyl acetate/hexanes, 24 g silica gel cartridge) to afford impure product (125 mg). This material was purified by reverse-phase preparative HPLC (Phenomenex® Luna® C8(2) 5 μm 100 Å AXIA™ column (30 mm×150 mm). gradient of acetonitrile (A) and 0.1% trifluoroacetic acid in water (B), 50 mL/minute (0-0.5 minutes 10% A, 0.5-7.0 minutes linear gradient 10-95% A, 7.0-10.0 minutes 95% A, 10.0-12.0 minutes linear gradient 95-10% A)) to afford the title compound (65 mg, 0.171 mmol, 28.6% yield). MS(APCI+) m/z 380 (M+H)⁺. This material was purified via reverse-phase chromatography to afford the title compound (65 mg, 0.171 mmol, 28.6% yield). MS(APCI+) m/z 380 (M+H)⁺.

### Example 21B

### 2-(2-{[2-(difluoromethoxy)pyridin-3-yl]oxy}-5-ethylphenyl)-N-(2-methylquinoline-5-sulfonyl)oxolane-2-carboxamide

To a solution of Example 21A (55 mg, 0.145 mmol) in dichloromethane (1 mL) was added 4-dimethylaminopyridine (35.4 mg, 0.290 mmol) and 1-(3-dimethylaminopropyl)-3-ethylcarbodiimide hydrochloride (55.6 mg, 0.290 mmol). The solution was stirred at ambient temperature for 20 minutes, then 2-methylquinoline-5-sulfonamide (32.2 mg, 0.145 mmol) was added, and the stirring continued for 2 hours. The reaction volume was reduced by about half under a stream of nitrogen, and the reaction quenched with 2 N aqueous citric acid (0.5 mL). The resulting residue was purified by flash chromatography (0-100% (3:1 ethyl acetate/ethanol)/hexanes, 12 g silica gel cartridge) to afford the title compound (75 mg). ¹H NMR (600 MHz, CDCl₃) δ ppm 9.31 (s, 1H), 8.78 (dd, J=8.9, 0.8 Hz, 1H), 8.39 (dd, J=7.5, 1.2 Hz, 1H), 8.24 (dt, J=8.4, 1.1 Hz, 1H), 7.85 (dd, J=4.9, 1.6 Hz, 1H), 7.73 (dd, J=8.4, 7.4 Hz, 1H), 7.56-7.41 (m, 1H), 7.31 (d, J=8.9 Hz, 1H), 7.18 (d, J=2.2 Hz, 1H), 6.99 (ddt, J=8.3, 2.3, 0.7 Hz, 1H), 6.86 (dd, J=7.9, 4.9 Hz, 1H), 6.68 (dd, J=7.9, 1.6 Hz, 1H), 6.44 (d, J=8.3 Hz, 1H), 3.91-3.84 (m, 2H), 2.78-2.74 (m, 1H), 2.73 (s, 3H), 2.57-2.50 (m, 2H), 2.32 (dt, J=13.2, 8.1 Hz, 1H), 1.94 (dddt, J=10.5, 8.1, 7.1, 5.3 Hz, 1H), 1.82 (dp, J=12.4, 7.8 Hz, 1H), 1.17 (t, J=7.6 Hz, 3H). MS(APCI+) m/z 583 (M+H)⁺.

### Example 22

### (2S)-2-(5-ethyl-2-phenoxyphenyl)-N-(2-methylquinoline-5-sulfonyl)oxolane-2-carboxamide

The enantiomers of Example 17 (92 mg) were separated by chiral preparative supercritical fluid chromatography (Chiralpak IC column (21×250 mm, 5 micron), 21 mg/mL in methanol, 44 g/minutes CO₂, RT 2.1 minutes) to provide the title compound (45 mg). ¹H NMR (600 MHz, CDCl₃) δ ppm 9.10 (s, 1H), 8.71 (dd, J=8.8, 0.9 Hz, 1H), 8.43 (dd, J=7.5, 1.2 Hz, 1H), 8.25 (dt, J=8.4, 1.1 Hz, 1H), 7.74 (dd, J=8.4, 7.4 Hz, 1H), 7.26 (d, J=3.4 Hz, 1H), 7.20 (d, J=2.2 Hz, 1H), 7.16-7.08 (m, 2H), 7.03-6.95 (m, 2H), 6.55-6.45 (m, 3H), 3.98-3.89 (m, 2H), 2.72 (s, 3H), 2.63 (ddd, J=13.4, 8.0, 5.6 Hz, 1H), 2.54 (q, J=7.6 Hz, 2H), 2.21 (dt, J=13.2, 7.8 Hz, 1H), 2.00-1.82 (m, 2H), 1.18 (t, J=7.6 Hz, 3H). MS(APCI+) m/z 517 (M+H)⁺.

### Example 23

### (2R)-2-(5-ethyl-2-phenoxyphenyl)-N-(2-methylquinoline-5-sulfonyl)oxolane-2-carboxamide

The enantiomers of Example 17 (92 mg) were separated by chiral preparative supercritical fluid chromatography (Chiralpak IC column (21×250 mm, 5 micron), 21 mg/mL in methanol, 44 g/minutes CO₂, RT 3.6 minutes) to provide the title compound (41 mg). ¹H NMR (600 MHz, CDCl₃) δ ppm 9.10 (s, 1H), 8.71 (dd, J=8.8, 0.9 Hz, 1H), 8.43 (dd, J=7.5, 1.2 Hz, 1H), 8.25 (dt, J=8.4, 1.1 Hz, 1H), 7.74 (dd, J=8.4, 7.4 Hz, 1H), 7.26 (d, J=3.4 Hz, 1H), 7.20 (d, J=2.2 Hz, 1H), 7.16-7.08 (m, 2H), 7.03-6.95 (m, 2H), 6.55-6.45 (m, 3H), 3.98-3.89 (m, 2H), 2.72 (s, 3H), 2.63 (ddd, J=13.4, 8.0, 5.6 Hz, 1H), 2.54 (q, J=7.6 Hz, 2H), 2.21 (dt, J=13.2, 7.8 Hz, 1H), 2.00-1.82 (m, 2H), 1.18 (t, J=7.6 Hz, 3H). MS(APCI+) m/z 517 (M+H)⁺.

### Example 24

### (2S)-2-{5-ethyl-2-[(2-methoxypyridin-3-yl)oxy]phenyl}-N-(2-methylquinoline-5-sulfonyl)oxolane-2-carboxamide

The enantiomers of Example 18 (65 mg) were separated by chiral preparative supercritical fluid chromatography (Chiralpak IC column (21×250 mm, 5 micron), 21 mg/mL in methanol, 56 g/minutes CO₂, RT 3.8 minutes) to provide the title compound (19 mg). ¹H NMR (500 MHz, CDCl₃) δ ppm 9.83 (s, 1H), 8.87 (dd, J=8.9, 0.8 Hz, 1H), 8.40 (dd, J=7.4, 1.2 Hz, 1H), 8.21 (dt, J=8.5, 1.1 Hz, 1H), 7.87 (dd, J=4.5, 2.1 Hz, 1H), 7.71 (dd, J=8.5, 7.5 Hz, 1H), 7.29-7.25 (m, 1H), 7.11 (d, J=2.2 Hz, 1H), 6.97 (dd, J=8.3, 2.2 Hz, 1H), 6.76-6.65 (m, 2H), 6.51 (d, J=8.3 Hz, 1H), 4.04 (s, 3H), 3.88 (td, J=8.1, 5.1 Hz, 1H), 3.79 (q, J=7.6 Hz, 1H), 2.82-2.74 (m, 1H), 2.72 (s, 3H), 2.51 (q, J=7.6 Hz, 2H), 2.24 (dt, J=12.9, 8.4 Hz, 1H), 1.96-1.86 (m, 1H), 1.86-1.77 (m, 1H), 1.14 (t, J=7.6 Hz, 3H). MS(APCI+) m/z 483 (M+H)⁺.

### Example 25

### (2R)-2-{5-ethyl-2-[(2-methoxypyridin-3-yl)oxy]phenyl}-N-(2-methylquinoline-5-sulfonyl)oxolane-2-carboxamide

The enantiomers of Example 18 (65 mg) were separated by chiral preparative supercritical fluid chromatography (Chiralpak IC column (21×250 mm, 5 micron,), 21 mg/mL in methanol, 56 g/minutes CO₂, RT 5.3 minutes) to provide the title compound (19 mg). ¹H NMR (500 MHz, CDCl₃) δ ppm 9.83 (s, 1H), 8.87 (dd, J=8.9, 0.8 Hz, 1H), 8.40 (dd, J=7.4, 1.2 Hz, 1H), 8.21 (dt, J=8.5, 1.1 Hz, 1H), 7.87 (dd, J=4.5, 2.1 Hz, 1H), 7.71 (dd, J=8.5, 7.5 Hz, 1H), 7.29-7.25 (m, 1H), 7.11 (d, J=2.2 Hz, 1H), 6.97 (dd, J=8.3, 2.2 Hz, 1H), 6.76-6.65 (m, 2H), 6.51 (d, J=8.3 Hz, 1H), 4.04 (s, 3H), 3.88 (td, J=8.1, 5.1 Hz, 1H), 3.79 (q, J=7.6 Hz, 1H), 2.82-2.74 (m, 1H), 2.72 (s, 3H), 2.51 (q, J=7.6 Hz, 2H), 2.24 (dt, J=12.9, 8.4 Hz, 1H), 1.96-1.86 (m, 1H), 1.86-1.77 (m, 1H), 1.14 (t, J=7.6 Hz, 3H). MS(APCI+) m/z 483 (M+H)⁺.

### Example 26

### (2S)-2-(2-{[2-(difluoromethoxy)pyridin-3-yl]oxy}-5-ethylphenyl)-N-(2-methylquinoline-5-sulfonyl)oxolane-2-carboxamide

The enantiomers of Example 21 (74 mg) were separated by chiral preparative supercritical fluid chromatography (Chiralpak IC column (21×250 mm, 5 micron), 18 mg/mL in methanol, 70 g/minutes CO₂, RT 2.6 minutes) to provide the title compound (28 mg). ¹H NMR (600 MHz, CDCl₃) δ ppm 9.31 (s, 1H), 8.78 (dd, J=8.9, 0.8 Hz, 1H), 8.39 (dd, J=7.5, 1.2 Hz, 1H), 8.24 (dt, J=8.4, 1.1 Hz, 1H), 7.85 (dd, J=4.9, 1.6 Hz, 1H), 7.73 (dd, J=8.4, 7.4 Hz, 1H), 7.56-7.41 (m, 1H), 7.31 (d, J=8.9 Hz, 1H), 7.18 (d, J=2.2 Hz, 1H), 6.99 (ddt, J=8.3, 2.3, 0.7 Hz, 1H), 6.86 (dd, J=7.9, 4.9 Hz, 1H), 6.68 (dd, J=7.9, 1.6 Hz, 1H), 6.44 (d, J=8.3 Hz, 1H), 3.91-3.84 (m, 2H), 2.78-2.74 (m, 1H), 2.73 (s, 3H), 2.57-2.50 (m, 2H), 2.32 (dt, J=13.2, 8.1 Hz, 1H), 1.94 (dddt, J=10.5, 8.1, 7.1, 5.3 Hz, 1H), 1.82 (dp, J=12.4, 7.8 Hz, 1H), 1.17 (t, J=7.6 Hz, 3H). MS(APCI+) m/z 583 (M+H)⁺.

### Example 27

### (2R)-2-(2-{[2-(difluoromethoxy)pyridin-3-yl]oxy}-5-ethylphenyl)-N-(2-methylquinoline-5-sulfonyl)oxolane-2-carboxamide

The enantiomers of Example 21 (74 mg) were separated by chiral preparative supercritical fluid chromatography (Chiralpak IC column (21×250 mm, 5 micron), 18 mg/mL in methanol, 70 g/minutes CO₂, RT 3.6 minutes) to provide the title compound (34 mg). ¹H NMR (600 MHz, CDCl₃) δ ppm 9.31 (s, 1H), 8.78 (dd, J=8.9, 0.8 Hz, 1H), 8.39 (dd, J=7.5, 1.2 Hz, 1H), 8.24 (dt, J=8.4, 1.1 Hz, 1H), 7.85 (dd, J=4.9, 1.6 Hz, 1H), 7.73 (dd, J=8.4, 7.4 Hz, 1H), 7.56-7.41 (m, 1H), 7.31 (d, J=8.9 Hz, 1H), 7.18 (d, J=2.2 Hz, 1H), 6.99 (ddt, J=8.3, 2.3, 0.7 Hz, 1H), 6.86 (dd, J=7.9, 4.9 Hz, 1H), 6.68 (dd, J=7.9, 1.6 Hz, 1H), 6.44 (d, J=8.3 Hz, 1H), 3.91-3.84 (m, 2H), 2.78-2.74 (m, 1H), 2.73 (s, 3H), 2.57-2.50 (m, 2H), 2.32 (dt, J=13.2, 8.1 Hz, 1H), 1.94 (dddt, J=10.5, 8.1, 7.1, 5.3 Hz, 1H), 1.82 (dp, J=12.4, 7.8 Hz, 1H), 1.17 (t, J=7.6 Hz, 3H). MS(APCI+) m/z 583 (M+H)⁺.

### Example 28

### (2R,4S)-4-methoxy-2-(2-methoxy-5-methylphenyl)-N-(2-methylquinoline-5-sulfonyl)oxolane-2-carboxamide

### Example 28A

### (R)-methyl 2-(2-methoxy-5-methylphenyl)-4-oxotetrahydrofuran-2-carboxylate

The enantiomers of Example 15C (23.8 g) were separated by chiral preparative supercritical fluid chromatography (Whelk-O (S,S) column (21×250 mm, 5 micron) 40 mg/mL in methanol, 60 g/minutes CO₂, RT 5.5 minutes) to provide the title compound (8.5 g). ¹H NMR (500 MHz, dimethyl sulfoxide-d₆) δ ppm 7.38 (dd, J=2.3, 0.8 Hz, 1H), 7.14 (ddd, J=8.2, 2.3, 0.8 Hz, 1H), 6.95 (d, J=8.2 Hz, 1H), 4.33 (dt, J=16.6, 1.1 Hz, 1H), 4.07 (d, J=16.6 Hz, 1H), 3.71 (s, 3H), 3.62 (s, 3H), 3.28 (dt, J=17.9, 0.7 Hz, 1H), 2.57 (dd, J=18.0, 1.2 Hz, 1H), 2.30 (s, 3H). MS(APCI+) m/z 265 (M+H)⁺.

### Example 28B

### (2R,4S)-methyl 4-hydroxy-2-(2-methoxy-5-methylphenyl)tetrahydrofuran-2-carboxylate

To a solution of Example 28A (0.479 g, 1.813 mmol) in dichloromethane (10 mL) cooled with an ice bath was added sodium tetrahydroborate (0.151 g, 3.99 mmol) in portions. After 2 hours, the reaction was quenched with saturated aqueous ammonium chloride (15 mL), and the aqueous layer was extracted with ethyl acetate (3×50 mL). The combined organic layers were dried over anhydrous sodium sulfate, filtered, and concentrated under reduced pressure. The crude product was purified by flash chromatography (0-50% tert-butyl methyl ether in hexanes, 40 g silica gel cartridge) to provide the title compound (0.325 g, 1.220 mmol, 67.3% yield) as the first eluting isomer. ¹H NMR (500 MHz, CDCl₃) δ ppm 7.34 (dd, J=2.3, 0.7 Hz, 1H), 7.14-7.03 (m, 1H), 6.76 (d, J=8.2 Hz, 1H), 4.49-4.39 (m, 1H), 4.15-4.09 (m, 2H), 4.06 (d, J=10.2 Hz, 1H), 3.75 (s, 3H), 3.69 (s, 3H), 3.10 (dt, J=14.9, 1.1 Hz, 1H), 2.31 (t, J=0.6 Hz, 3H), 2.07 (ddd, J=14.8, 6.0, 0.7 Hz, 1H). MS(APCI+) m/z 267 (M+H)⁺.

### Example 28C

### (2R,4S)-methyl 4-methoxy-2-(2-methoxy-5-methylphenyl)tetrahydrofuran-2-carboxylate

To a solution of Example 28B (0.187 g, 0.702 mmol) in N,N-dimethylformamide (2.341 mL) was added sodium hydride (43 mg, 1.075 mmol, 60% dispersion in mineral oil) in portions. After 30 minutes, iodomethane (0.088 mL, 1.404 mmol) was added, and the reaction stirred at ambient temperature for 16 hours. More iodomethane (0.088 mL, 1.404 mmol) was added and after 2 hours, more sodium hydride (0.056 g, 1.404 mmol) was added. After 1 hour more, the reaction was quenched with saturated aqueous ammonium chloride (2 mL), diluted with water (10 mL) and tert-butyl methyl ether (100 mL). The aqueous layer was separated, and the organic layers were washed with water, washed with brine, dried over anhydrous sodium sulfate, filtered, and concentrated under reduced pressure. The crude product was purified by flash chromatography (0-100% tert-butyl methyl ether in hexanes, 12 g silica gel cartridge) to provide the title compound (0.187 g, 0.667 mmol, 95% yield). ¹H NMR (500 MHz, CDCl₃) δ ppm 7.33 (dd, J=2.4, 0.8 Hz, 1H), 7.07 (ddq, J=8.2, 2.3, 0.7 Hz, 1H), 6.76 (d, J=8.2 Hz, 1H), 4.17-4.10 (m, 1H), 4.07-4.00 (m, 2H), 3.76 (s, 3H), 3.68 (s, 3H), 3.35 (s, 3H), 3.29-3.22 (m, 1H), 2.31 (d, J=0.7 Hz, 3H), 2.19 (dd, J=14.0, 6.3 Hz, 1H). MS(APCI+) m/z 281 (M+H)⁺.

### Example 28D

### (2R,4S)-4-methoxy-2-(2-methoxy-5-methylphenyl)tetrahydrofuran-2-carboxylic Acid

To a solution of Example 28C (176 mg, 0.628 mmol) in tetrahydrofuran (1 mL) and methanol (1 mL) was added 3 N aqueous sodium hydroxide (1.046 mL, 3.14 mmol). The solution was stirred at ambient temperature for 72 hours, then acidified with 2.0 M aqueous citric acid (0.9 mL). The aqueous layer was extracted with dichloromethane (3×4 mL) on a 25 mL Isolute phase separator, and the organic layers concentrated to afford the title compound (0.169 g, 0.635 mmol, 101% yield). ¹H NMR (400 MHz, CDCl₃) δ ppm 7.26 (d, J=2.6 Hz, 1H), 7.13-7.08 (m, 1H), 6.83 (d, J=8.3 Hz, 1H), 4.26 (ddd, J=9.9, 1.7, 1.0 Hz, 1H), 4.13-4.08 (m, 1H), 3.94 (dd, J=9.9, 3.3 Hz, 1H), 3.82 (s, 3H), 3.39 (s, 3H), 3.10 (dt, J=14.5, 1.6 Hz, 1H), 2.46 (dd, J=14.5, 4.8 Hz, 1H), 2.29 (s, 3H). MS(APCI+) m/z 267 (M+H)⁺.

### Example 28E

### (2R,4S)-4-methoxy-2-(2-methoxy-5-methylphenyl)-N-(2-methylquinoline-5-sulfonyl)oxolane-2-carboxamide

A solution of Example 28D (166 mg, 0.623 mmol), 1-(3-dimethylaminopropyl)-3-ethylcarbodiimide hydrochloride (239 mg, 1.247 mmol), and 4-dimethylaminopyridine (95 mg, 0.779 mmol) in anhydrous dichloromethane (3 mL) was stirred at ambient temperature for 30 minutes, and then 2-methylquinoline-5-sulfonamide (139 mg, 0.623 mmol) was added. The reaction was stirred at ambient temperature for 2 hours, then quenched with 2.0 M aqueous citric acid (1 mL), and the organic layers were purified by flash chromatography (0-100% (3:1 ethyl acetate:ethanol)/hexanes, 24 g silica gel cartridge) to afford the title compound (0.246 g, 0.523 mmol, 84% yield). ¹H NMR (400 MHz, dimethyl sulfoxide-d₆) δ ppm 11.77 (s, 1H), 8.81 (d, J=8.9 Hz, 1H), 8.27-8.18 (m, 2H), 7.89-7.81 (m, 1H), 7.48 (d, J=8.9 Hz, 1H), 7.04-6.96 (m, 2H), 6.66 (d, J=8.9 Hz, 1H), 3.94-3.78 (m, 3H), 3.29 (s, 3H), 2.78 (s, 3H), 2.75-2.72 (m, 1H), 2.68 (s, 3H), 2.09 (s, 3H), 2.05 (dd, J=14.1, 5.2 Hz, 1H). MS(APCI+) m/z 471 (M+H)⁺.

### Example 29

### (2R,4R)-4-methoxy-2-(2-methoxy-5-methylphenyl)-N-(2-methylquinoline-5-sulfonyl)oxolane-2-carboxamide

### Example 29A

### (2R,4R)-methyl 4-hydroxy-2-(2-methoxy-5-methylphenyl)tetrahydrofuran-2-carboxylate

To a solution of Example 28A (0.479 g, 1.813 mmol) in dichloromethane (10 mL) cooled by an ice bath was added sodium tetrahydroborate (0.151 g, 3.99 mmol) in portions. After 2 hours, the reaction was quenched with saturated aqueous ammonium chloride (15 mL), and the aqueous layer was extracted with ethyl acetate (3×50 mL). The combined organic layers were dried over anhydrous sodium sulfate, filtered, and concentrated under reduced pressure. The crude product was purified by flash chromatography (0-50% tert-butyl methyl ether in hexanes, 40 g silica gel cartridge) to provide the title compound (0.155 g, 0.582 mmol, 32.1% yield) as the second eluting isomer. ¹H NMR (500 MHz, CDCl₃) δ ppm 7.50 (dd, J=2.4, 0.7 Hz, 1H), 7.11-7.06 (m, 1H), 6.76 (d, J=8.2 Hz, 1H), 4.60 (dddd, J=8.9, 6.0, 3.9, 1.9 Hz, 1H), 4.11 (dt, J=9.7, 1.7 Hz, 1H), 4.05 (dd, J=9.7, 3.9 Hz, 1H), 3.75 (s, 3H), 3.65 (s, 3H), 3.37 (dd, J=14.2, 6.0 Hz, 1H), 2.32 (d, J=0.7 Hz, 3H), 2.04 (ddd, J=14.2, 2.2, 1.6 Hz, 1H), 1.52 (d, J=7.1 Hz, 1H). MS(APCI+) m/z 267 (M+H)⁺.

### Example 29B

### (2R,4R)-methyl 4-methoxy-2-(2-methoxy-5-methylphenyl)tetrahydrofuran-2-carboxylate

To a solution of Example 29A (0.140 g, 0.526 mmol) in N,N-dimethylformamide (1.752 mL) was added sodium hydride (0.042 g, 1.050 mmol, 60% dispersion in mineral oil) in portions. After 30 minutes, iodomethane (0.066 mL, 1.051 mmol) was added, and the reaction stirred at ambient temperature for 16 hours. Additional iodomethane (0.066 mL, 1.051 mmol) was added and after 2 hours, and then more sodium hydride (0.042 g, 1.050 mmol, 60% dispersion in mineral oil) added. After 1 hour, the reaction was diluted with ethyl acetate (100 mL) and quenched with saturated aqueous ammonium chloride (15 mL), and the aqueous layer was separated. The organic layer was washed with water, washed with brine, dried over anhydrous sodium sulfate, filtered, and concentrated under reduced pressure. The crude product was purified by flash chromatography (0-100% tert-butyl methyl ether in hexanes, 10 g silica gel cartridge) to provide the title compound (102 mg, 0.364 mmol, 69.2% yield). ¹H NMR (600 MHz, CDCl₃) δ ppm 7.48-7.43 (m, 1H), 7.05 (ddq, J=8.2, 2.2, 0.7 Hz, 1H), 6.74 (d, J=8.2 Hz, 1H), 4.26-4.20 (m, 1H), 4.13 (ddd, J=9.7, 2.7, 1.2 Hz, 1H), 4.01 (dd, J=9.7, 4.9 Hz, 1H), 3.74 (s, 3H), 3.65 (s, 3H), 3.32 (dd, J=13.9, 6.8 Hz, 1H), 3.24 (s, 3H), 2.31 (d, J=0.7 Hz, 3H), 1.94 (ddd, J=13.9, 3.6, 1.2 Hz, 1H). MS(APCI+) m/z 281 (M+H)⁺.

### Example 29C

### (2R,4R)-4-methoxy-2-(2-methoxy-5-methylphenyl)tetrahydrofuran-2-carboxylic Acid

To a solution of Example 29B (95 mg, 0.339 mmol) in tetrahydrofuran (0.6 mL) and methanol (0.6 mL) was added 3 N aqueous sodium hydroxide (0.565 mL, 1.695 mmol). The solution was stirred at ambient temperature for 72 hours, and the reaction was acidified with 2.0 M aqueous citric acid (0.9 mL). The aqueous layer was extracted with dichloromethane (3×3 mL) on a 25 mL Isolute phase separator, and the combined organic layers were concentrated under reduced pressure to afford the title compound (89 mg, 0.334 mmol, 99% yield). ¹H NMR (500 MHz, CDCl₃) δ ppm 7.46-7.42 (m, 1H), 7.08 (ddd, J=8.2, 2.2, 0.8 Hz, 1H), 6.78 (d, J=8.3 Hz, 1H), 4.19 (ddt, J=6.8, 4.8, 3.4 Hz, 1H), 4.13-4.05 (m, 2H), 3.78 (s, 3H), 3.26 (s, 3H), 3.16 (dd, J=14.0, 6.8 Hz, 1H), 2.31 (d, J=0.7 Hz, 3H), 2.18 (ddd, J=14.0, 3.7, 0.9 Hz, 1H). MS(APCI+) m/z 267 (M+H)⁺.

### Example 29D

### (2R,4R)-4-methoxy-2-(2-methoxy-5-methylphenyl)-N-(2-methylquinoline-5-sulfonyl)oxolane-2-carboxamide

A solution of Example 29C (89 mg, 0.334 mmol), 1-(3-dimethylaminopropyl)-3-ethylcarbodiimide hydrochloride (128 mg, 0.668 mmol), and 4-dimethylaminopyridine (51.0 mg, 0.418 mmol) in anhydrous dichloromethane (1.5 mL) was stirred at ambient temperature for 30 minutes, and then 2-methylquinoline-5-sulfonamide (74.3 mg, 0.334 mmol) was added. The reaction was stirred at ambient temperature for 2 hours, and the reaction was quenched with 2.0 M aqueous citric acid (0.5 mL). The organic layer was purified by flash chromatography (0-100% of (3:1 ethyl acetate/ethanol)/hexanes, 10 g silica gel cartridge) to afford the desired product (112 mg). The product was triturated with methanol, then ether, and then ethyl acetate/ethanol to afford the title compound (90 mg, 0.191 mmol, 57.2% yield). ¹H NMR (500 MHz, dimethyl sulfoxide-d₆) δ ppm 11.95 (s, 1H), 8.71 (d, J=8.9 Hz, 1H), 8.26-8.19 (m, 2H), 7.88 (t, J=7.9 Hz, 1H), 7.45 (d, J=8.9 Hz, 1H), 7.32 (d, J=2.2 Hz, 1H), 7.03 (dd, J=8.2, 2.3 Hz, 1H), 6.58 (d, J=8.2 Hz, 1H), 3.87 (ddt, J=10.8, 7.8, 2.4 Hz, 2H), 3.36-3.26 (m, 1H), 3.05 (s, 3H), 3.01 (s, 3H), 2.93 (dd, J=14.0, 6.9 Hz, 1H), 2.71 (s, 3H), 2.27 (s, 3H), 1.66-1.56 (m, 1H). MS(APCI+) m/z 471 (M+H)⁺.

### Example 30

### (2R,4S)-4-methoxy-2-(2-methoxy-5-methylphenyl)-4-methyl-N-(2-methylquinoline-5-sulfonyl)oxolane-2-carboxamide

### Example 30A

### (2R,4S)-methyl 4-hydroxy-2-(2-methoxy-5-methylphenyl)-4-methyltetrahydrofuran-2-carboxylate

To a solution of Example 28A (0.266 g, 1.007 mmol) in diethyl ether (3 mL) and tetrahydrofuran (3.0 mL) at −78° C. was added methylmagnesium bromide (0.336 mL, 1.007 mmol, 3 M in diethyl ether) dropwise, and the reaction was allowed reach ambient temperature slowly and stirred for 16 hours. The reaction mixture was quenched with saturated aqueous ammonium chloride (2 mL), diluted with water, and extracted with ethyl acetate (3×50 mL). The combined organic layers were washed with brine, dried over anhydrous sodium sulfate, filtered, and concentrated. The crude product was purified by flash chromatography (0-100% tert-butyl methyl ether/hexanes/ethyl acetate, 20 g silica gel cartridge) to afford the title compound (0.125 g, 0.446 mmol, 44.3% yield). ¹H NMR (500 MHz, CDCl₃) δ ppm 7.36 (dd, J=2.3, 0.7 Hz, 1H), 7.08 (ddt, J=8.1, 2.2, 0.7 Hz, 1H), 6.76 (d, J=8.2 Hz, 1H), 4.43 (d, J=1.0 Hz, 1H), 4.05 (dd, J=8.9, 1.6 Hz, 1H), 3.86 (d, J=8.9 Hz, 1H), 3.74 (s, 3H), 3.69 (s, 3H), 3.15 (dd, J=14.5, 1.6 Hz, 1H), 2.32 (s, 3H), 1.97 (d, J=14.5 Hz, 1H), 1.35 (s, 3H). MS(APCI+) m/z 281 (M+H)⁺.

### Example 30B

### (2R,4S)-methyl 4-methoxy-2-(2-methoxy-5-methylphenyl)-4-methyltetrahydrofuran-2-carboxylate

To a solution of Example 30A (0.111 g, 0.396 mmol) in N,N-dimethylformamide (1.320 mL) was added sodium hydride (0.079 g, 1.980 mmol, 60% dispersion in mineral oil) in portions. After 30 minutes, iodomethane (0.124 mL, 1.980 mmol) was added, and the reaction stirred at ambient temperature for 2.5 hours. The reaction was diluted with ethyl acetate (100 mL) and quenched with saturated aqueous ammonium chloride (15 mL) and the aqueous layer was separated. The organic layer was washed with water, washed with brine, dried over anhydrous sodium sulfate, filtered, and concentrated under reduced pressure. The crude product was purified by flash chromatography (0-100% tert-butyl methyl ether in hexanes, 10 g silica gel cartridge) to provide the title compound (50 mg, 0.170 mmol, 42.9% yield). ¹H NMR (500 MHz, CDCl₃) δ ppm 7.40 (dd, J=2.3, 0.7 Hz, 1H), 7.06 (ddt, J=7.6, 2.3, 0.7 Hz, 1H), 6.76 (d, J=8.3 Hz, 1H), 4.09 (dd, J=9.0, 1.1 Hz, 1H), 3.78 (dd, J=8.9, 0.5 Hz, 1H), 3.75 (s, 3H), 3.67 (s, 3H), 3.51 (dd, J=13.9, 1.1 Hz, 1H), 3.31 (s, 3H), 2.31 (t, J=0.7 Hz, 3H), 1.86 (dd, J=13.9, 0.5 Hz, 1H), 1.28 (s, 3H). MS(APCI+) m/z 281 (M+H)⁺.

### Example 30C

### (2R,4S)-4-methoxy-2-(2-methoxy-5-methylphenyl)-4-methyltetrahydrofuran-2-carboxylic Acid

To a solution of Example 30B (46 mg, 0.156 mmol) in tetrahydrofuran (0.3 mL) and methanol (0.3 mL) was added 3 N aqueous sodium hydroxide (0.260 mL, 0.781 mmol). The solution was stirred at ambient temperature for 72 hours, and then acidified with 2.0 M aqueous citric acid (0.5 mL). The aqueous layer was extracted with dichloromethane (3×1 mL) on a 25 mL Isolute phase separator, and the organic layer was concentrated under reduced pressure to afford the title compound. ¹H NMR (400 MHz, CDCl₃) δ ppm 10.21 (s, 1H), 7.28 (d, J=2.3 Hz, 1H), 7.10 (dd, J=8.3, 2.2 Hz, 1H), 6.83 (d, J=8.3 Hz, 1H), 4.24 (dd, J=9.6, 2.0 Hz, 1H), 3.81 (s, 3H), 3.66 (d, J=9.6 Hz, 1H), 3.32 (s, 3H), 3.17 (dd, J=14.5, 2.0 Hz, 1H), 2.29 (s, 3H), 2.27 (d, J=14.7 Hz, 1H), 1.34 (s, 3H). MS(APCI+) m/z 281 (M+H)⁺.

### Example 30D

### (2R,4S)-4-methoxy-2-(2-methoxy-5-methylphenyl)-4-methyl-N-(2-methylquinoline-5-sulfonyl)oxolane-2-carboxamide

A solution of Example 30C (40 mg, 0.143 mmol), 1-(3-dimethylaminopropyl)-3-ethylcarbodiimide hydrochloride (54.7 mg, 0.285 mmol), and 4-dimethylaminopyridine (21.79 mg, 0.178 mmol) in anhydrous dichloromethane (1.5 mL) was stirred at ambient temperature for 30 minutes, and then 2-methylquinoline-5-sulfonamide (31.7 mg, 0.143 mmol) was added. The reaction was stirred at ambient temperature for 2 hours, and then the reaction was quenched with 2.0 M aqueous citric acid (0.5 mL). The organic layer was were purified by flash chromatography (0-100% of (3:1 ethyl acetate/ethanol)/hexanes, 10 g silica gel cartridge) and then purified by reverse-phase preparative HPLC (Phenomenex® Luna® C8(2) 5 μm 100 Å AXIA™ column (30 mm×150 mm); gradient of acetonitrile (A) and 0.1% trifluoroacetic acid in water (B), 50 mL/minute (0-0.5 minutes 10% A, 0.5-7.0 minutes linear gradient 10-95% A, 7.0-10.0 minutes 95% A, 10.0-12.0 minutes linear gradient 95-10% A)) to afford the title compound as a trifluoroacetic acid salt (55 mg, 0.092 mmol, 64.4% yield). ¹H NMR (400 MHz, dimethyl sulfoxide-d₆) δ ppm 11.80 (s, 1H), 8.92 (d, J=8.9 Hz, 1H), 8.33-8.22 (m, 2H), 7.91 (dd, J=8.5, 7.4 Hz, 1H), 7.57 (d, J=8.9 Hz, 1H), 7.03-6.92 (m, 2H), 6.68 (d, J=8.2 Hz, 1H), 3.88 (dd, J=9.0, 1.3 Hz, 1H), 3.60 (d, J=9.0 Hz, 1H), 3.38 (s, 3H), 2.94 (dd, J=14.0, 1.4 Hz, 1H), 2.73 (s, 3H), 2.66 (s, 3H), 2.05 (s, 3H), 1.88 (d, J=13.8 Hz, 1H), 1.12 (s, 3H). MS(APCI+) m/z 485 (M+H)⁺.

### Example 31

### (2S,4R)-4-methoxy-2-(2-methoxy-5-methylphenyl)-4-methyl-N-(2-methylquinoline-5-sulfonyl)oxolane-2-carboxamide

### Example 31A

### (S)-methyl 2-(2-methoxy-5-methylphenyl)-4-oxotetrahydrofuran-2-carboxylate

The enantiomers of Example 15C (23.8 g) were separated by chiral preparative supercritical fluid chromatography (Whelk-O (S,S) column (21×250 mm, 5 micron, 40 mg/mL in methanol, 60 g/minutes CO₂, RT 6.0 minutes) to provide the title compound (5.4 g). ¹H NMR (500 MHz, dimethyl sulfoxide-d₆) δ ppm 7.38 (dd, J=2.3, 0.8 Hz, 1H), 7.14 (ddd, J=8.2, 2.3, 0.8 Hz, 1H), 6.95 (d, J=8.2 Hz, 1H), 4.33 (dt, J=16.6, 1.1 Hz, 1H), 4.07 (d, J=16.6 Hz, 1H), 3.71 (s, 3H), 3.62 (s, 3H), 3.28 (dt, J=17.9, 0.7 Hz, 1H), 2.57 (dd, J=18.0, 1.2 Hz, 1H), 2.30 (s, 3H). MS(APCI+) m/z 265 (M+H)⁺.

### Example 31B

### (2S,4R)-methyl 4-hydroxy-2-(2-methoxy-5-methylphenyl)-4-methyltetrahydrofuran-2-carboxylate

To a solution of Example 31A (0.495 g, 1.873 mmol) in tetrahydrofuran (6 mL) at −73° C. was added methylmagnesium bromide (0.624 mL, 1.873 mmol, 3 M in diethyl ether) dropwise, and the reaction was allowed reach ambient temperature slowly and then stirred at ambient temperature for 16 hours. The reaction mixture was quenched with saturated aqueous ammonium chloride (2 mL), diluted with water, and extracted with ethyl acetate (3×50 mL). The combined organic layers were washed with brine, dried over anhydrous sodium sulfate, filtered, and concentrated. The crude product was purified by flash chromatography (0-100% tert-butyl methyl ether/hexanes, 40 g silica gel cartridge) to afford the title compound (0.176 g, 0.628 mmol, 33.5% yield). ¹H NMR (500 MHz, CDCl₃) δ ppm 7.36 (dd, J=2.3, 0.8 Hz, 1H), 7.08 (ddq, J=8.2, 2.3, 0.7 Hz, 1H), 6.76 (d, J=8.2 Hz, 1H), 4.43 (d, J=1.1 Hz, 1H), 4.05 (dd, J=8.9, 1.6 Hz, 1H), 3.86 (d, J=8.9 Hz, 1H), 3.74 (s, 3H), 3.69 (s, 3H), 3.15 (dd, J=14.5, 1.6 Hz, 1H), 2.32 (t, J=0.7 Hz, 3H), 1.97 (d, J=14.4 Hz, 1H), 1.35 (d, J=0.9 Hz, 3H). MS(APCI+) m/z 281 (M+H)⁺.

### Example 31C

### (2S,4R)-methyl 4-methoxy-2-(2-methoxy-5-methylphenyl)-4-methyltetrahydrofuran-2-carboxylate

To a solution of (2S,4R)-methyl 4-hydroxy-2-(2-methoxy-5-methylphenyl)-4-methyltetrahydrofuran-2-carboxylate (0.102 g, 0.364 mmol) Example 31B in N,N-dimethylformamide (1.213 mL) was added sodium hydride (0.073 g, 1.819 mmol, 60% dispersion in mineral oil) in portions. After 30 minutes, iodomethane (0.114 mL, 1.819 mmol) was added and the reaction stirred at ambient temperature for 72 hours. The reaction was diluted with tert-butyl methyl ether (100 mL) and quenched with saturated aqueous ammonium chloride (15 mL), and the aqueous layer was separated. The organic layer was washed with water, washed with brine, dried over anhydrous sodium sulfate, filtered, and concentrated under reduced pressure. The crude product was purified by flash chromatography (0-100% tert-butyl methyl ether in hexanes, 10 g silica gel cartridge) to provide the title compound (54.2 mg, 0.184 mmol, 50.6% yield). ¹H NMR (500 MHz, CDCl₃) δ ppm 7.40 (dd, J=2.3, 0.7 Hz, 1H), 7.06 (ddt, J=7.6, 2.3, 0.7 Hz, 1H), 6.76 (d, J=8.3 Hz, 1H), 4.09 (dd, J=9.0, 1.1 Hz, 1H), 3.78 (dd, J=8.9, 0.5 Hz, 1H), 3.75 (s, 3H), 3.67 (s, 3H), 3.51 (dd, J=13.9, 1.1 Hz, 1H), 3.31 (s, 3H), 2.31 (t, J=0.7 Hz, 3H), 1.86 (dd, J=13.9, 0.5 Hz, 1H), 1.28 (s, 3H). MS(APCI+) m/z 295 (M+H)⁺.

### Example 31D

### (2S,4R)-4-methoxy-2-(2-methoxy-5-methylphenyl)-4-methyltetrahydrofuran-2-carboxylic Acid

To a solution of Example 31C (54 mg, 0.183 mmol) in tetrahydrofuran (0.3 mL) and methanol (0.3 mL) was added 3 N aqueous sodium hydroxide (0.306 mL, 0.917 mmol). The solution was stirred at ambient temperature for 72 hours, and then the reaction was acidified with 2.0 M aqueous citric acid (0.5 mL). The aqueous layer was extracted with dichloromethane (3×1 mL) on a 25 mL Isolute phase separator, and the organic layer was concentrated under reduced pressure to afford the title compound (50 mg, 0.178 mmol, 97% yield). ¹H NMR (500 MHz, CDCl₃) δ ppm 10.15 (s, 1H), 7.28 (dd, J=2.4, 0.7 Hz, 1H), 7.13-7.08 (m, 1H), 6.83 (d, J=8.3 Hz, 1H), 4.24 (dd, J=9.7, 2.0 Hz, 1H), 3.81 (s, 3H), 3.66 (d, J=9.7 Hz, 1H), 3.32 (s, 3H), 3.17 (dd, J=14.5, 2.0 Hz, 1H), 2.29 (d, J=0.7 Hz, 3H), 2.27 (d, J=14.5 Hz, 1H), 1.34 (s, 3H). MS(APCI+) m/z 281 (M+H)⁺.

### Example 31E

### (2S,4R)-4-methoxy-2-(2-methoxy-5-methylphenyl)-4-methyl-N-(2-methylquinoline-5-sulfonyl)oxolane-2-carboxamide

A solution of Example 31D (50 mg, 0.178 mmol), 1-(3-dimethylaminopropyl)-3-ethylcarbodiimide hydrochloride (68.4 mg, 0.357 mmol), and 4-dimethylaminopyridine (27.2 mg, 0.223 mmol) in anhydrous dichloromethane (1.5 mL) was stirred at ambient temperature for 30 minutes, and then 2-methylquinoline-5-sulfonamide (39.6 mg, 0.178 mmol) was added. The reaction was stirred at ambient temperature for 2 hours, and then quenched with 2.0 M aqueous citric acid (0.5 mL). The organic layer was purified by flash chromatography (0-100% (3:1 ethyl acetate/ethanol)/hexanes, 10 g silica gel cartridge) and then purified by reverse-phase preparative HPLC (Phenomenex® Luna® C8(2) 5 μm 100 Å AXIA™ column (30 mm×150 mm); gradient of acetonitrile (A) and 0.1% trifluoroacetic acid in water (B), 50 mL/minute (0-0.5 minutes 10% A, 0.5-7.0 minutes linear gradient 10-95% A, 7.0-10.0 minutes 95% A, 10.0-12.0 minutes linear gradient 95-10% A)) to afford the title compound as a trifluoroacetic acid salt (75 mg, 0.125 mmol, 70.2% yield). ¹H NMR (500 MHz, dimethyl sulfoxide-d₆) δ ppm 11.79 (s, 1H), 8.89 (dd, J=8.9, 0.9 Hz, 1H), 8.40-8.16 (m, 2H), 7.90 (dd, J=8.5, 7.4 Hz, 1H), 7.55 (d, J=8.9 Hz, 1H), 7.14-6.90 (m, 2H), 6.69 (d, J=8.3 Hz, 1H), 3.88 (dd, J=9.0, 1.4 Hz, 1H), 3.61 (d, J=8.9 Hz, 1H), 3.38 (s, 3H), 2.94 (dd, J=13.9, 1.4 Hz, 1H), 2.72 (s, 3H), 2.67 (s, 3H), 2.05 (d, J=0.7 Hz, 3H), 1.88 (d, J=13.9 Hz, 1H), 1.12 (s, 3H). MS(APCI+) m/z 485 (M+H)⁺.

### Example 32

### (2R,4S)-2-(2-methoxy-5-methylphenyl)-N-(2-methylquinoline-5-sulfonyl)-4-phenyloxolane-2-carboxamide

### Example 32A

### (2R,4R)-methyl 4-hydroxy-2-(2-methoxy-5-methylphenyl)-4-phenyltetrahydrofuran-2-carboxylate

To a solution of Example 28A (0.250 g, 0.946 mmol) and lanthanum(III) chloride bis(lithium chloride) complex (1.577 mL, 0.946 mmol, 0.6 M in tetrahydrofuran) in tetrahydrofuran (4.73 mL) and cooled by a dry ice/acetone bath was added phenylmagnesium bromide (1.230 mL, 1.230 mmol, 1 M in tetrahydrofuran) in tetrahydrofuran dropwise, keeping the internal temperature below −70° C. The mixture was allowed to warm to 0° C. over 2 hours. The reaction was quenched with saturated aqueous ammonium chloride (20 mL), diluted with water, and extracted with ethyl acetate (3×50 mL). The combined organic layers were washed with brine, dried over anhydrous sodium sulfate, and concentrated. The crude product was purified by flash chromatography (0-50% tert-butyl methyl ether/hexanes, 20 g silica gel cartridge) to afford the title compound (0.296 g, 0.865 mmol, 91% yield). ¹H NMR (500 MHz, CDCl₃) δ ppm 7.49 (dd, J=1.9, 1.5 Hz, 1H), 7.48 (t, J=1.1 Hz, 1H), 7.45 (dd, J=2.3, 0.8 Hz, 1H), 7.35-7.29 (m, 2H), 7.26-7.21 (m, 1H), 7.10 (ddt, J=8.2, 2.3, 0.8 Hz, 1H), 6.78 (d, J=8.2 Hz, 1H), 4.89 (s, 1H), 4.31 (dd, J=9.1, 1.7 Hz, 1H), 4.26 (d, J=9.1 Hz, 1H), 3.74 (s, 3H), 3.73 (s, 3H), 3.44 (dd, J=14.7, 1.7 Hz, 1H), 2.44 (d, J=14.7 Hz, 1H), 2.35 (t, J=0.7 Hz, 3H). MS(APCI+) m/z 325 (M−OH)⁺.

### Example 32B

### (2R)-methyl 2-(2-methoxy-5-methylphenyl)-4-phenyltetrahydrofuran-2-carboxylate

To a solution of Example 32A (0.296 g, 0.865 mmol) and triethylsilane (0.276 mL, 1.729 mmol) in dichloromethane (4 mL) at about −7° C. was added 2,2,2-trifluoroacetic acid (0.733 mL, 9.51 mmol), maintaining the internal temperature at −7° C. The reaction was allowed to warm to ambient temperature. After about an hour, saturated aqueous sodium bicarbonate was added. The organic layer taken up in dichloromethane, passed through an aqueous/organic separator tube, and concentrated under reduced pressure. The crude material was purified by flash chromatography (0-30% tert-butyl methyl ether/hexanes, 40 g silica gel cartridge) to afford the title compound (0.167 g, 0.512 mmol, 59.2% yield). MS(APCI+) m/z 327 (M+H)⁺.

### Example 32C

### (2R)-2-(2-methoxy-5-methylphenyl)-4-phenyltetrahydrofuran-2-carboxylic Acid

To a solution of Example 32B (149.9 mg, 0.459 mmol) in tetrahydrofuran (2.0 mL) and methanol (2.0 mL), was added 3 N aqueous sodium hydroxide (0.75 mL, 2.250 mmol). The solution was heated at 40° C. for 30 minutes, stirred over the weekend at ambient temperature, and then heated at 40° C. for 2 hours. The reaction was acidified with 1.0 M aqueous citric acid (4 mL), extracted with dichloromethane (3×10 mL) on a 25 mL Isolute phase separator. The organic layer was concentrated to afford the title compound (168.2 mg, 0.538 mmol, 117% yield). MS(APCI+) m/z 313 (M+H)⁺.

### Example 32D

### (2R)-2-(2-methoxy-5-methylphenyl)-N-((2-methylquinolin-5-yl)sulfonyl)-4-phenyltetrahydrofuran-2-carboxamide

To a solution of Example 32C (168.2 mg, 0.538 mmol), 1-(3-dimethylaminopropyl)-3-ethylcarbodiimide hydrochloride (206 mg, 1.077 mmol), and 4-dimethylaminopyridine (82 mg, 0.673 mmol) in anhydrous dichloromethane (5.0 mL) was added 2-methylquinoline-5-sulfonamide (120 mg, 0.538 mmol). The mixture was heated for 2 hours at 38° C. and then quenched with 1.0 M aqueous citric acid (4 mL) and extracted with dichloromethane (2×10 mL) on a 25 mL Isolute phase separator. The material was purified by flash chromatography (0-50% 3:1 ethyl acetate/ethanol in dichloromethane over 25 minutes, 40 mL/minute, RediSep® Rf Gold 40 g column) to afford the title compound (142.8 mg, 0.276 mmol, 51.3% yield). ¹H NMR (500 MHz, dimethyl sulfoxide-d₆) δ ppm 12.07 (s, 1H), 8.80 (d, J=8.9 Hz, 1H), 8.27 (d, J=7.3 Hz, 1H), 8.24 (d, J=8.5 Hz, 1H), 7.90 (t, J=7.9 Hz, 1H), 7.48 (d, J=8.9 Hz, 1H), 7.28-7.22 (m, 3H), 7.21-7.16 (m, 1H), 7.11-7.06 (m, 2H), 7.06-7.01 (m, 1H), 6.63 (d, J=8.3 Hz, 1H), 4.15 (t, J=7.9 Hz, 1H), 3.73 (t, J=8.7 Hz, 1H), 3.21-3.14 (m, 1H), 3.11 (s, 3H), 3.05 (dd, J=13.4, 7.0 Hz, 1H), 2.71 (s, 3H), 2.22 (s, 3H), 1.85 (t, J=11.8 Hz, 1H). MS(APCI+) m/z 517 (M+H)⁺.

### Example 32E

### (2R,4S)-2-(2-methoxy-5-methylphenyl)-N-(2-methylquinoline-5-sulfonyl)-4-phenyloxolane-2-carboxamide

The enantiomers of Example 32D (140 mg) were separated by chiral preparative supercritical fluid chromatography (ES Industries AD-H column (21×250 mm, 5 micron 12.8 mg/mL in 10:1 methanol/diethylamine, 56 g/minutes CO₂, RT 8.0 minutes) to provide the title compound (39.9 mg, 0.077 mmol, 28.3% yield). ¹H NMR (500 MHz, dimethyl sulfoxide-d₆) δ ppm 12.07 (s, 1H), 8.80 (d, J=8.9 Hz, 1H), 8.27 (d, J=7.3 Hz, 1H), 8.24 (d, J=8.5 Hz, 1H), 7.90 (t, J=7.9 Hz, 1H), 7.48 (d, J=8.9 Hz, 1H), 7.28-7.22 (m, 3H), 7.21-7.16 (m, 1H), 7.11-7.06 (m, 2H), 7.06-7.01 (m, 1H), 6.63 (d, J=8.3 Hz, 1H), 4.15 (t, J=7.9 Hz, 1H), 3.73 (t, J=8.7 Hz, 1H), 3.21-3.14 (m, 1H), 3.11 (s, 3H), 3.05 (dd, J=13.4, 7.0 Hz, 1H), 2.71 (s, 3H), 2.22 (s, 3H), 1.85 (t, J=11.8 Hz, 1H). MS(APCI+) m/z 517 (M+H)⁺.

### Example 33

### (2R,4R)-2-(2-methoxy-5-methylphenyl)-N-(2-methylquinoline-5-sulfonyl)-4-phenyloxolane-2-carboxamide

The enantiomers of Example 32D (140 mg) were separated by chiral preparative supercritical fluid chromatography (ES Industries AD-H column (21×250 mm, 5 micron) 12.8 mg/mL in 10:1 methanol/diethylamine, 56 g/minutes CO₂, RT 11.8 minutes) to provide the title compound (84.7 mg, 0.164 mmol, 60.2% yield). ¹H NMR (500 MHz, dimethyl sulfoxide-d₆) δ ppm 12.07 (s, 1H), 8.80 (d, J=8.9 Hz, 1H), 8.27 (d, J=7.3 Hz, 1H), 8.24 (d, J=8.5 Hz, 1H), 7.90 (t, J=7.9 Hz, 1H), 7.48 (d, J=8.9 Hz, 1H), 7.28-7.22 (m, 3H), 7.21-7.16 (m, 1H), 7.11-7.06 (m, 2H), 7.06-7.01 (m, 1H), 6.63 (d, J=8.3 Hz, 1H), 4.15 (t, J=7.9 Hz, 1H), 3.73 (t, J=8.7 Hz, 1H), 3.21-3.14 (m, 1H), 3.11 (s, 3H), 3.05 (dd, J=13.4, 7.0 Hz, 1H), 2.71 (s, 3H), 2.22 (s, 3H), 1.85 (t, J=11.8 Hz, 1H). MS(APCI+) m/z 517 (M+H)⁺.

### Example 34

### (2S)—N-(2-aminoquinoline-5-sulfonyl)-2-(2-methoxy-5-methylphenyl)-4,4-dimethyloxolane-2-carboxamide

### Example 34A

### methyl 2-(3-bromo-2,2-dimethylpropoxy)-2-(2-methoxy-5-methylphenyl)acetate

A mixture of Example 3B (1.0970 g, 4.98 mmol), 3-bromo-2,2-dimethylpropan-1-ol (1.2935 g, 7.74 mmol), and dichloromethane (10 mL) was subjected to blue light (420 nM, 50% intensity) in a Penn M2 photoreactor for 4 hours. The reaction was concentrated and purified by flash chromatography (0-50% ethyl acetate/hexanes, 40 g silica gel cartridge) to afford the title compound (1.558 g, 4.34 mmol, 87% yield). ¹H NMR (500 MHz, CDCl₃) δ ppm 7.20 (d, J=2.3 Hz, 1H), 7.11-7.07 (m, 1H), 6.80 (d, J=8.4 Hz, 1H), 5.26 (s, 1H), 3.82 (s, 3H), 3.70 (s, 3H), 3.46 (d, J=9.8 Hz, 1H), 3.42 (d, J=6.8 Hz, 1H), 3.40 (d, J=5.9 Hz, 1H), 3.28 (d, J=8.9 Hz, 1H), 2.29 (d, J=0.7 Hz, 3H), 1.06 (s, 3H), 1.02 (s, 3H).

### Example 34B

### methyl 2-(2-methoxy-5-methylphenyl)-4,4-dimethyltetrahydrofuran-2-carboxylate

To a mixture of Example 34A (1.558 g, 4.34 mmol) in tetrahydrofuran (43.4 mL) under nitrogen at −75° C. was added sodium bis(trimethylsilyl)amide (1 M in tetrahydrofuran, 6.07 mL, 6.07 mmol) dropwise over 5 minutes. The reaction was stirred at −75° C. for 30 minutes and then allowed to warm to ambient temperature. After 3 hours, the reaction was adsorbed directly onto silica gel and purified by flash column chromatography (0-50% ethyl acetate/hexanes, 80 g silica gel cartridge) to afford the title compound (0.486 g, 1.746 mmol, 40.3% yield). ¹H NMR (500 MHz, CDCl₃) δ ppm 7.47 (dd, J=2.3, 0.7 Hz, 1H), 7.11-7.00 (m, 1H), 6.74 (d, J=8.2 Hz, 1H), 3.75-3.68 (m, 5H), 3.64 (s, 3H), 3.04 (d, J=13.2 Hz, 1H), 2.32 (d, J=0.8 Hz, 3H), 1.75 (dd, J=13.2, 0.7 Hz, 1H), 1.17 (s, 3H), 1.02 (s, 3H).

### Example 34C

### 2-(2-methoxy-5-methylphenyl)-4,4-dimethyltetrahydrofuran-2-carboxylic Acid

To a solution of Example 34B (0.465 g, 1.671 mmol) in tetrahydrofuran (3 mL) and methanol (3 mL) was added 3 N sodium hydroxide (2.78 mL, 8.35 mmol). The solution was stirred at 45° C. for 3 hours and then at ambient temperature overnight. The mixture was concentrated under reduced pressure and acidified with 2.0 M citric acid (5 mL). The aqueous layer was decanted, and the residue washed with water. The resulting precipitate was filtered, washed with water, and washed with hexanes to afford the title compound (0.356 g, 1.347 mmol, 81% yield). ¹H NMR (600 MHz, CDCl₃) δ ppm 7.42 (dd, J=2.2, 0.8 Hz, 1H), 7.10-7.07 (m, 1H), 6.79 (d, J=8.3 Hz, 1H), 3.79 (s, 3H), 3.75 (d, J=8.3 Hz, 1H), 3.73-3.70 (m, 1H), 2.81 (d, J=13.4 Hz, 1H), 2.31 (d, J=0.7 Hz, 3H), 2.07 (d, J=13.4 Hz, 1H), 1.16 (s, 3H), 1.05 (s, 3H). MS (APCI+) m/z 265 (M+H)⁺.

### Example 34D

### (R)-4,4-dimethyl-2-oxotetrahydrofuran-3-yl (S)-2-(2-methoxy-5-methylphenyl)-4,4-dimethyltetrahydrofuran-2-carboxylate

A solution of 34C (316 mg, 1.196 mmol), 1-(3-dimethylaminopropyl)-3-ethylcarbodiimide hydrochloride (458 mg, 2.391 mmol), and 4-(dimethylamino)pyridine (4-dimethylaminopyridine) (183 mg, 1.494 mmol) in dry dichloromethane (3 mL) was stirred at ambient temperature for 30 minutes. D-(−)-Pantolactone (190 mg, 1.460 mmol) was added, and the reaction was stirred at ambient temperature for 3 hours. The reaction was quenched with 2.0 M citric acid (2.5 mL), and the organic layer was purified by flash chromatography (0-50% of tert-butyl methyl ether/hexanes, 40 g silica gel cartridge) to afford the title compound (0.200 g, 0.531 mmol, 44.4% yield). ¹H NMR (500 MHz, CDCl₃) δ ppm 7.43 (dd, J=2.3, 0.8 Hz, 1H), 7.06-7.01 (m, 1H), 6.73 (d, J=8.2 Hz, 1H), 5.25 (s, 1H), 3.93 (s, 2H), 3.81-3.73 (m, 5H), 2.96 (d, J=13.5 Hz, 1H), 2.31 (d, J=0.8 Hz, 3H), 1.91 (dd, J=13.4, 0.7 Hz, 1H), 1.19 (s, 3H), 1.15 (s, 3H), 1.05 (s, 3H), 0.93 (s, 3H). MS (APCI+) m/z 377 (M+H)⁺.

### Example 34E

### (R)-4,4-dimethyl-2-oxotetrahydrofuran-3-yl (R)-2-(2-methoxy-5-methylphenyl)-4,4-dimethyltetrahydrofuran-2-carboxylate

Example 34E was isolated as the second peak in the purification of Example 34D (0.178 mg, 0.473 mmol, 39.6% yield). ¹H NMR (600 MHz, CDCl₃) δ ppm 7.53 (dd, J=2.3, 0.8 Hz, 1H), 7.06-7.02 (m, 1H), 6.71 (d, J=8.2 Hz, 1H), 5.28 (s, 1H), 3.94 (s, 2H), 3.78-3.74 (m, 2H), 3.74 (s, 3H), 3.04 (d, J=13.4 Hz, 1H), 2.31 (d, J=0.7 Hz, 3H), 1.85 (dd, J=13.5, 0.7 Hz, 1H), 1.18 (s, 3H), 1.08 (s, 3H), 1.02 (s, 3H), 0.76 (s, 3H).

### Example 34F

### (S)-2-(2-methoxy-5-methylphenyl)-4,4-dimethyltetrahydrofuran-2-carboxylic Acid

A mixture of Example 34D (178 mg, 0.473 mmol) and lithium hydroxide (76 mg, 3.17 mmol) in dioxane (1.5 mL) and water (0.500 mL) was stirred at 80° C. overnight. The reaction was concentrated under reduced pressure and then acidified with 2.0 M citric acid (2 mL). The aqueous layer was put through an aqueous/organic separator tube with dichloromethane. The organic layer was concentrated to afford the title compound (147 mg, 0.556 mmol, 118% yield). ¹H NMR (400 MHz, CDCl₃) δ ppm 7.42 (d, J=2.2 Hz, 1H), 7.12-7.06 (m, 1H), 6.80 (d, J=8.3 Hz, 1H), 3.81 (s, 3H), 3.77 (d, J=8.3 Hz, 1H), 3.72 (d, J=8.4 Hz, 1H), 2.80 (d, J=13.4 Hz, 1H), 2.31 (s, 3H), 2.12 (d, J=13.4 Hz, 1H), 1.17 (s, 3H), 1.06 (s, 3H). MS (APCI+) m/z 265 (M+H)⁺.

### Example 34G

### (S)—N-((2-chloroquinolin-5-yl)sulfonyl)-2-(2-methoxy-5-methylphenyl)-4,4-dimethyltetrahydrofuran-2-carboxamide

To a solution of Example 34F (79.3 mg, 0.30 mmol) in dichloromethane (0.2 mL) was added a solution of 1-ethyl-3-[3-(dimethylamino)propyl]-carbodiimide hydrochloride (69 mg, 0.36 mmol) and 4-(dimethylamino)pyridine (73.3 mg, 0.6 mmol) in dichloromethane (0.6 mL). A slurry of 2-chloroquinoline-5-sulfonamide (80.1 mg, 0.33 mmol) in dichloromethane (0.5 mL) was added, and the reaction was stirred overnight at ambient temperature. The reaction mixture was concentrated under a stream of nitrogen. The residue was reconstituted in dimethyl sulfoxide/methanol and purified via reverse-phase HPLC (Phenomenex® Luna® C8(2) 5 μm 100 Å AXIA column (50 mm×30 mm) column, gradient of acetonitrile (A) and 0.1% trifluoroacetic acid in water (B), 40 mL/minutes (0-0.5 minutes 15% A, 0.5-8.0 minutes linear gradient 15-100% A, 8.0-9.0 minutes 100% A, 9.0-9.1 minutes linear gradient 100-15% A, 9.1-10 minutes 15% A)) to afford the title compound (121 mg, 67% yield). ¹H NMR (400 MHz, dimethyl sulfoxide) δ ppm 11.93 (s, 1H), 8.77 (dd, J=9.0, 0.8 Hz, 1H), 8.30 (dd, J=7.5, 1.3 Hz, 1H), 8.22 (dt, J=8.5, 1.0 Hz, 1H), 7.96 (dd, J=8.5, 7.4 Hz, 1H), 7.54 (d, J=9.1 Hz, 1H), 7.24 (d, J=2.3 Hz, 1H), 6.98-6.91 (m, 1H), 6.46 (d, J=8.3 Hz, 1H), 3.49 (d, J=8.2 Hz, 1H), 3.20 (d, J=8.3 Hz, 1H), 3.02 (s, 3H), 2.61 (d, J=13.3 Hz, 1H), 2.18 (s, 3H), 1.45 (d, J=13.2 Hz, 1H), 0.84 (s, 3H), 0.73 (s, 3H). MS (APCI+) m/z 489.2 (M+H)⁺.

### Example 34H

### (2S)—N-(2-aminoquinoline-5-sulfonyl)-2-(2-methoxy-5-methylphenyl)-4,4-dimethyloxolane-2-carboxamide

To a mixture of Example 34G (121 mg, 0.25 mmol) and 1,4-dioxane (0.25 mL) was added (methanesulfonato-κO)[2′-(methylamino)-2-biphenylyl]palladium-dicyclohexyl(2′,6′-dimethoxy-2-biphenylyl)phosphine (1:1) (SPhos Pd G4) (9.9 mg, 0.125 mmol) in dioxane (0.124 mL). Lithium hexamethyldisilazide (0.548 mL, 1 M in tetrahydrofuran) was added, and the reaction was heated for 2 hours at 80° C. The reaction was cooled to ambient temperature, quenched with acetic acid (0.1 mL), and purified via reverse-phase HPLC (Phenomenex® Luna® C8(2) 5 μm 100 Å AXIA column (50 mm×30 mm), gradient of acetonitrile (A) and 0.1% trifluoroacetic acid in water (B), 40 mL/minutes (0-0.5 minutes 15% A, 0.5-8.0 minutes linear gradient 15-100% A, 8.0-9.0 minutes 100% A, 9.0-9.1 minutes linear gradient 100-15% A, 9.1-10 minutes 15% A)) to afford the title compound (65.2 mg, 45% yield). ¹H NMR (400 MHz, dimethyl sulfoxide) δ ppm 8.59 (d, J=9.8 Hz, 1H), 8.49 (s, 2H), 8.02 (dd, J=6.9, 1.8 Hz, 1H), 7.93-7.81 (m, 2H), 7.32 (d, J=2.3 Hz, 1H), 7.01 (dd, J=8.3, 2.3 Hz, 1H), 6.92 (d, J=9.7 Hz, 1H), 6.60 (d, J=8.3 Hz, 1H), 3.56 (d, J=8.2 Hz, 1H), 3.29 (d, J=8.3 Hz, 1H), 3.20 (s, 3H), 2.69 (d, J=13.3 Hz, 1H), 2.25 (s, 3H), 1.51 (d, J=13.3 Hz, 1H), 0.90 (s, 3H), 0.83 (s, 3H). MS (APCI+) m/z 470.2 (M+H)⁺.

### Example 35

### (2R)—N-(2-aminoquinoline-5-sulfonyl)-2-(2-methoxy-5-methylphenyl)-4,4-dimethyloxolane-2-carboxamide

### Example 35A

### (R)-2-(2-methoxy-5-methylphenyl)-4,4-dimethyltetrahydrofuran-2-carboxylic Acid

A mixture of Example 34E (200 mg, 0.531 mmol) and lithium hydroxide (82 mg, 3.42 mmol) in dioxane (1.5 mL) and water (0.500 mL) was stirred at 80° C. overnight. The reaction was concentrated under reduced pressure, and the crude reaction was acidified with 2.0 M citric acid (2 mL). The aqueous layer was put through an aqueous/organic separator tube with dichloromethane. The organic layer was concentrated to afford the title compound (0.145 g, 0.549 mmol, 103% yield). ¹H NMR (400 MHz, CDCl₃) δ ppm 7.42 (d, J=2.2 Hz, 1H), 7.12-7.06 (m, 1H), 6.80 (d, J=8.3 Hz, 1H), 3.81 (s, 3H), 3.77 (d, J=8.3 Hz, 1H), 3.72 (d, J=8.4 Hz, 1H), 2.80 (d, J=13.4 Hz, 1H), 2.31 (s, 3H), 2.12 (d, J=13.4 Hz, 1H), 1.17 (s, 3H), 1.06 (s, 3H). MS (APCI+) m/z 265 (M+H)⁺.

### Example 35B

### (R)—N-((2-chloroquinolin-5-yl)sulfonyl)-2-(2-methoxy-5-methylphenyl)-4,4-dimethyltetrahydrofuran-2-carboxamide

To a solution of Example 35A (79.3 mg, 0.30 mmol) in dichloromethane (0.2 mL) was added a solution of 1-ethyl-3-[3-(dimethylamino)propyl]-carbodiimide hydrochloride (69 mg, 0.36 mmol) and 4-(dimethylamino)pyridine (73.3 mg, 0.6 mmol) in dichloromethane (0.6 mL). A slurry of 2-chloroquinoline-5-sulfonamide (80.1 mg, 0.33 mmol) in dichloromethane (0.5 mL) was added, and the reaction was stirred overnight at ambient temperature. The reaction mixture was concentrated under a stream of nitrogen. The residue was reconstituted in dimethyl sulfoxide/methanol and purified via reverse-phase HPLC (Phenomenex® Luna® C8(2) 5 μm 100 Å AXIA column (50 mm×30 mm) column using a gradient of acetonitrile (A) and 0.1% trifluoroacetic acid in water (B), 40 mL/minutes (0-0.5 minutes 15% A, 0.5-8.0 minutes linear gradient 15-100% A, 8.0-9.0 minutes 100% A, 9.0-9.1 minutes linear gradient 100-15% A, 9.1-10 minutes 15% A)) to afford the title compound (138 mg, 76% yield). ¹H NMR (400 MHz, dimethyl sulfoxide) δ ppm 11.93 (s, 1H), 8.77 (d, J=9.2 Hz, 1H), 8.30 (d, J=7.4 Hz, 1H), 8.22 (d, J=8.4 Hz, 1H), 7.95 (dd, J=8.5, 7.4 Hz, 1H), 7.54 (d, J=9.1 Hz, 1H), 7.24 (d, J=2.3 Hz, 1H), 6.94 (dd, J=8.2, 2.2 Hz, 1H), 6.46 (d, J=8.3 Hz, 1H), 3.49 (d, J=8.3 Hz, 1H), 3.20 (d, J=8.2 Hz, 1H), 3.02 (s, 3H), 2.61 (d, J=13.3 Hz, 1H), 2.18 (s, 3H), 1.44 (d, J=13.3 Hz, 1H), 0.84 (s, 3H), 0.73 (s, 3H). MS (APCI+) m/z 489.2 (M+H)⁺.

### Example 35C

### (2R)—N-(2-aminoquinoline-5-sulfonyl)-2-(2-methoxy-5-methylphenyl)-4,4-dimethyloxolane-2-carboxamide

To a mixture of Example 35B (138 mg, 0.28 mmol) and 1,4-dioxane (0.28 mL) was added (methanesulfonato-κO)[2′-(methylamino)-2-biphenylyl]palladium-dicyclohexyl(2′,6′-dimethoxy-2-biphenylyl)phosphine (1:1) (SPhos Pd G4) (10.3 mg, 0.14 mmol) in dioxane (0.14 mL). Lithium hexamethyldisilazide (0.620 mL, 1 M in tetrahydrofuran) was added, and the reaction was heated for 2 hours at 80° C. The reaction was cooled to ambient temperature, quenched with acetic acid (0.1 mL) and purified via reverse-phase HPLC (Phenomenex® Luna® C8(2) 5 μm 100 Å AXIA column (50 mm×30 mm), gradient of acetonitrile (A) and 0.1% trifluoroacetic acid in water (B), 40 mL/minutes (0-0.5 minutes 15% A, 0.5-8.0 minutes linear gradient 15-100% A, 8.0-9.0 minutes 100% A, 9.0-9.1 minutes linear gradient 100-15% A, 9.1-10 minutes 15% A)) to afford the title compound (65.2 mg, 45% yield). ¹H NMR (400 MHz, dimethyl sulfoxide) δ ppm 8.58 (d, J=9.6 Hz, 1H), 8.01 (d, J=7.2 Hz, 1H), 7.91-7.80 (m, 2H), 7.32 (d, J=2.3 Hz, 1H), 7.01 (dd, J=8.4, 2.2 Hz, 1H), 6.90 (d, J=9.8 Hz, 1H), 6.60 (d, J=8.2 Hz, 1H), 3.56 (d, J=8.2 Hz, 1H), 3.29 (d, J=8.2 Hz, 1H), 3.20 (s, 3H), 2.68 (d, J=13.3 Hz, 1H), 2.25 (s, 3H), 1.52 (d, J=13.3 Hz, 1H), 0.89 (s, 3H), 0.83 (s, 3H). MS (APCI+) m/z 470.2 (M+H)⁺.

### Example 36

### (2S,4S)-2-(2-methoxy-5-methylphenyl)-N-(2-methylquinoline-5-sulfonyl)-4-phenyloxolane-2-carboxamide

### Example 36A

### (2S,4S)-methyl 4-hydroxy-2-(2-methoxy-5-methylphenyl)-4-phenyltetrahydrofuran-2-carboxylate

To a solution of Example 31A (0.202 g, 0.764 mmol) and lanthanum(III) chloride bis(lithium chloride) complex (1.274 mL, 0.764 mmol, 0.6 M in tetrahydrofuran) in tetrahydrofuran (3.82 mL) at cooled −78° C. was added phenylmagnesium bromide, (0.994 mL, 0.994 mmol, 1 M in tetrahydrofuran) dropwise, and the reaction was allowed to warm to 0° C. The reaction was diluted with dichloromethane, quenched with saturated aqueous ammonium chloride (2 mL), diluted with water, and extracted with dichloromethane (3×25 mL). The combined organic layers were concentrated, and the crude product was purified by flash chromatography (0-50% tert-butyl methyl ether/hexanes, 20 g silica gel cartridge) to afford the title compound (0.256 g, 0.748 mmol, 98% yield). ¹H NMR (500 MHz, CDCl₃) δ ppm 7.51-7.46 (m, 2H), 7.45 (dd, J=2.3, 0.7 Hz, 1H), 7.34-7.29 (m, 2H), 7.26-7.22 (m, 1H), 7.13-7.07 (m, 1H), 6.78 (d, J=8.2 Hz, 1H), 4.89 (s, 1H), 4.31 (dd, J=9.1, 1.7 Hz, 1H), 4.26 (d, J=9.1 Hz, 1H), 3.74 (s, 3H), 3.73 (s, 3H), 3.44 (dd, J=14.7, 1.7 Hz, 1H), 2.44 (d, J=14.7 Hz, 1H), 2.35 (s, 3H). MS(APCI+) m/z 325 (M−OH)⁺.

### Example 36B

### (2S,4S)-methyl 2-(2-methoxy-5-methylphenyl)-4-phenyltetrahydrofuran-2-carboxylate

To a solution of Example 36A (0.250 g, 0.730 mmol) and triethylsilane (0.233 mL, 1.460 mmol) in dichloromethane (4 mL) and cooled by an ice bath was added 2,2,2-trifluoroacetic acid (0.619 mL, 8.03 mmol), and the reaction was allowed to warm to ambient temperature and stirred for 16 hours. The reaction was concentrated and treated with saturated aqueous sodium bicarbonate. The organic layer taken up in dichloromethane and purified by flash chromatography (0-40% tert-butyl methyl ether/hexanes, 20 g silica gel cartridge) to afford the title compound (95 mg, 0.291 mmol, 39.9% yield) as a the first eluting component. ¹H NMR (400 MHz, CDCl₃) δ ppm 7.44 (d, J=2.2 Hz, 1H), 7.35-7.27 (m, 4H), 7.25-7.18 (m, 1H), 7.10 (ddd, J=8.2, 2.4, 0.8 Hz, 1H), 6.78 (d, J=8.2 Hz, 1H), 4.49-4.41 (m, 1H), 3.93 (dd, J=10.8, 8.3 Hz, 1H), 3.75 (s, 3H), 3.71 (s, 3H), 3.38 (tt, J=10.7, 7.4 Hz, 1H), 3.26 (dd, J=12.7, 11.6 Hz, 1H), 2.48-2.41 (m, 1H), 2.36 (d, J=0.7 Hz, 3H). MS(APCI+) m/z 327 (M+H)⁺.

### Example 36C

### (2S,4S)-2-(2-methoxy-5-methylphenyl)-4-phenyltetrahydrofuran-2-carboxylic Acid

To a solution of Example 36B (67 mg, 0.205 mmol) in tetrahydrofuran (0.5 mL) and methanol (0.5 mL), was added 3 N aqueous sodium hydroxide (0.205 mL, 1.026 mmol). The solution was stirred at 45° C. for 3 hours, concentrated, and acidified with 2.0 M aqueous citric acid (0.5 mL). The aqueous layer was decanted, and the residue washed with water. The resulting precipitate was filtered, washed with water, and washed with hexanes to afford the title compound (62 mg, 0.198 mmol, 97% yield). ¹H NMR (500 MHz, CDCl₃) δ ppm 7.43-7.39 (m, 1H), 7.38-7.29 (m, 4H), 7.27-7.22 (m, 1H), 7.15-7.10 (m, 1H), 6.82 (d, J=8.2 Hz, 1H), 4.43-4.37 (m, 1H), 3.96 (t, J=9.5 Hz, 1H), 3.80 (s, 3H), 3.52 (dq, J=18.4, 9.2, 8.5 Hz, 1H), 3.00-2.90 (m, 1H), 2.90-2.81 (m, 1H), 2.36 (s, 3H). MS(APCI+) m/z 313 (M+H)⁺.

### Example 36D

### (2S,4S)-2-(2-methoxy-5-methylphenyl)-N-(2-methylquinoline-5-sulfonyl)-4-phenyloxolane-2-carboxamide

To a solution of Example 36C (60 mg, 0.192 mmol), 1-(3-dimethylaminopropyl)-3-ethylcarbodiimide hydrochloride (73.6 mg, 0.384 mmol), and 4-dimethylaminopyridine (29.3 mg, 0.240 mmol) in anhydrous dichloromethane (1 mL) was added 2-methylquinoline-5-sulfonamide (42.7 mg, 0.192 mmol). The reaction was stirred at ambient temperature for 16 hours, quenched with 2.0 M aqueous citric acid (0.5 mL), and extracted with dichloromethane (2×10 mL) on an 8 mL Isolute phase separator. The organic layer was concentrated under reduced pressure, and the crude material purified by flash chromatography (0-100% (3:1 ethyl acetate/ethanol) in hexanes, RediSep® Rf Gold 10 g column) to afford the title compound (45 mg, 0.087 mmol, 45.3% yield). ¹H NMR (600 MHz, dimethyl sulfoxide-d₆) δ ppm 12.04 (s, 1H), 8.83 (d, J=8.9 Hz, 1H), 8.25 (dd, J=24.3, 7.9 Hz, 2H), 7.89 (t, J=7.9 Hz, 1H), 7.48 (d, J=8.8 Hz, 1H), 7.29 (d, J=2.2 Hz, 1H), 7.26-7.16 (m, 3H), 7.13-7.03 (m, 3H), 6.65 (d, J=8.3 Hz, 1H), 4.24 (t, J=8.0 Hz, 1H), 3.49 (dd, J=10.3, 8.4 Hz, 1H), 3.30-3.22 (m, 1H), 3.07 (s, 3H), 2.68 (s, 3H), 2.56-2.51 (m, 1H), 2.30 (dd, J=13.5, 8.6 Hz, 1H), 2.27 (s, 3H). MS(APCI+) m/z 517 (M+H)⁺.

### Example 37

### (2S,4R)-2-(2-methoxy-5-methylphenyl)-N-(2-methylquinoline-5-sulfonyl)-4-phenyloxolane-2-carboxamide

### Example 37A

### (2S,4R)-methyl 2-(2-methoxy-5-methylphenyl)-4-phenyltetrahydrofuran-2-carboxylate

To a solution of Example 36A (0.250 g, 0.730 mmol) and triethylsilane (0.233 mL, 1.460 mmol) in dichloromethane (4 mL) and cooled by an ice bath was added 2,2,2-trifluoroacetic acid (0.619 mL, 8.03 mmol). The reaction was allowed to warm to ambient temperature and stirred for 16 hours. The reaction was concentrated and treated with saturated aqueous sodium bicarbonate. The organic layer taken up in dichloromethane and purified by flash chromatography (0-40% tert-butyl methyl ether/hexanes, 20 g silica gel cartridge) to afford the title compound (36 mg, 0.11 mmol, 15% yield) as the second eluting component. ¹H NMR (400 MHz, CDCl₃) δ ppm 7.49 (d, J=2.2 Hz, 1H), 7.31-7.27 (m, 1H), 7.24-7.17 (m, 4H), 7.08 (dt, J=8.1, 1.4 Hz, 1H), 6.76 (d, J=8.2 Hz, 1H), 4.45 (t, J=7.7 Hz, 1H), 3.97 (dd, J=9.6, 8.0 Hz, 1H), 3.84 (ddd, J=17.5, 10.4, 7.5 Hz, 1H), 3.73 (s, 3H), 3.70 (d, J=0.6 Hz, 3H), 3.51 (dd, J=13.0, 7.5 Hz, 1H), 2.34 (s, 3H), 1.98 (dd, J=13.1, 10.8 Hz, 1H). MS(APCI+) m/z 327 (M+H)⁺.

### Example 37B

### (2S,4R)-2-(2-methoxy-5-methylphenyl)-4-phenyltetrahydrofuran-2-carboxylic Acid

To a solution of Example 37A (24 mg, 0.074 mmol) in tetrahydrofuran (0.5 mL) and methanol (0.5 mL), was added 3 N aqueous sodium hydroxide (0.074 mL, 0.368 mmol), and the solution was stirred at 45° C. for 3 hours. The reaction mixture was reduced in volume and acidified with 2.0 M aqueous citric acid (0.5 mL). The aqueous layer was decanted and the residue washed with water. The resulting precipitate was filtered, washed with water, and washed with hexanes to afford the title compound (22 mg, 0.070 mmol, 96% yield). ¹H NMR (500 MHz, CDCl₃) δ ppm 7.37 (d, J=2.2 Hz, 1H), 7.31-7.26 (m, 2H), 7.21-7.17 (m, 2H), 7.15-7.11 (m, 1H), 7.10-7.05 (m, 1H), 6.78 (d, J=8.3 Hz, 1H), 4.44 (t, J=8.2 Hz, 1H), 3.95-3.86 (m, 1H), 3.75 (s, 3H), 3.38-3.30 (m, 1H), 2.41-2.36 (m, 1H), 2.28 (s, 3H), 2.27-2.23 (m, 1H). MS(APCI+) m/z 313 (M+H)⁺.

### Example 37C

### (2S,4R)-2-(2-methoxy-5-methylphenyl)-N-(2-methylquinoline-5-sulfonyl)-4-phenyloxolane-2-carboxamide

To a solution of Example 37B (22 mg, 0.070 mmol), 1-(3-dimethylaminopropyl)-3-ethylcarbodiimide hydrochloride (27.0 mg, 0.141 mmol), and 4-dimethylaminopyridine (10.76 mg, 0.088 mmol) in anhydrous dichloromethane (0.5 mL) was added 2-methylquinoline-5-sulfonamide (15.65 mg, 0.070 mmol). The mixture was stirred at ambient temperature for 16 hours. The reaction was quenched with 1.0 M aqueous citric acid (0.3 mL), extracted with dichloromethane (2×10 mL) on an 8 mL Isolute phase separator. The organic layer was concentrated under reduced pressure, and the crude material was purified by flash chromatography (0-100% (3:1 ethyl acetate/ethanol) in hexanes, on a RediSep® Rf Gold 10 g column) to afford the title compound (26 mg, 0.050 mmol, 71.5% yield). ¹H NMR (400 MHz, dimethyl sulfoxide-d₆) δ ppm 12.06 (s, 1H), 8.78 (d, J=8.9 Hz, 1H), 8.31-8.17 (m, 2H), 7.89 (t, J=7.9 Hz, 1H), 7.47 (d, J=8.9 Hz, 1H), 7.28-7.21 (m, 3H), 7.21-7.14 (m, 1H), 7.11-6.98 (m, 3H), 6.62 (d, J=8.3 Hz, 1H), 4.14 (t, J=7.9 Hz, 1H), 3.72 (t, J=8.7 Hz, 1H), 3.21-3.10 (m, 1H), 3.09 (s, 3H), 3.06-2.96 (m, 1H), 2.69 (s, 3H), 2.20 (s, 3H), 1.84 (dd, J=14.4, 9.0 Hz, 1H). MS(APCI+) m/z 517 (M+H)⁺.

### Example 38

### (2R,4S)-2-(2-methoxy-5-methylphenyl)-4-(2-methoxyphenyl)-N-(2-methylquinoline-5-sulfonyl)oxolane-2-carboxamide

### Example 38A

### (2R,4R)-methyl 4-hydroxy-2-(2-methoxy-5-methylphenyl)-4-(2-methoxyphenyl)tetrahydrofuran-2-carboxylate

To a solution of Example 28A (0.276 g, 1.044 mmol) and lanthanum(III) chloride bis(lithium chloride) complex (1.74 mL, 1.044 mmol, 0.6 M in tetrahydrofuran) in tetrahydrofuran (5.2 mL) at −78° C. was added (2-methoxyphenyl)magnesium bromide (1.358 mL, 1.358 mmol, 1.0 M in tetrahydrofuran) dropwise, and the reaction was allowed to warm to 0° C. The reaction was diluted with dichloromethane, quenched with saturated aqueous ammonium chloride (2 mL), diluted with water, and extracted with dichloromethane (3×25 mL). The combined organic layers were concentrated, and the crude product was purified by flash chromatography (0-50% tert-butyl methyl ether/hexanes, a 20 g silica gel cartridge) to afford the title compound (0.380 g, 1.020 mmol, 98% yield). ¹H NMR (600 MHz, CDCl₃) δ ppm 7.55 (dd, J=7.7, 1.7 Hz, 1H), 7.44 (dd, J=2.3, 0.7 Hz, 1H), 7.24 (ddd, J=8.2, 7.4, 1.7 Hz, 1H), 7.08-7.05 (m, 1H), 6.94 (td, J=7.6, 1.1 Hz, 1H), 6.84 (dd, J=8.2, 1.1 Hz, 1H), 6.76 (d, J=8.2 Hz, 1H), 4.60 (s, 1H), 4.50 (d, J=8.9 Hz, 1H), 4.36 (dd, J=8.9, 1.4 Hz, 1H), 3.75 (s, 3H), 3.74 (s, 3H), 3.71 (s, 3H), 3.38 (dd, J=14.4, 1.4 Hz, 1H), 2.73 (d, J=14.3 Hz, 1H), 2.53 (dd, J=14.0, 1.9 Hz, 0H), 2.33 (d, J=0.7 Hz, 3H). MS(APCI+) m/z 355 (M−OH)⁺.

### Example 38B

### (2R,4S)-methyl 2-(2-methoxy-5-methylphenyl)-4-(2-methoxyphenyl)tetrahydrofuran-2-carboxylate

To a solution of Example 38A (0.380 g, 1.020 mmol) and triethylsilane (0.326 mL, 2.041 mmol) in dichloromethane (4 mL) cooled by an ice bath was added 2,2,2-trifluoroacetic acid (0.865 mL, 11.22 mmol), keeping the internal temperature below 3° C. The reaction was allowed to warm to ambient temperature, and saturated aqueous sodium bicarbonate was added. The organic layer taken up in dichloromethane, passed through an aqueous/organic separator tube, and concentrated under reduced pressure. The crude material was purified by flash chromatography (0-30% tert-butyl methyl ether/hexanes, 40 g silica gel cartridge) to afford the title compound (73 mg, 0.205 mmol, 20.07% yield) as the second eluting isomer. ¹H NMR (600 MHz, CDCl₃) δ ppm 7.47 (dd, J=2.1, 0.6 Hz, 1H), 7.17 (ddd, J=8.1, 7.4, 1.7 Hz, 1H), 7.15-7.12 (m, 1H), 7.09-7.04 (m, 1H), 6.89-6.82 (m, 2H), 6.76 (d, J=8.2 Hz, 1H), 4.47 (t, J=7.8 Hz, 1H), 4.16-4.08 (m, 1H), 3.91 (dd, J=9.2, 7.9 Hz, 1H), 3.81 (s, 3H), 3.74 (s, 3H), 3.70 (s, 3H), 3.41 (dd, J=12.9, 7.5 Hz, 1H), 2.33 (d, J=0.8 Hz, 3H), 2.06 (dd, J=12.9, 10.8 Hz, 1H). MS(APCI+) m/z 357 (M+H)⁺.

### Example 38C

### (2R,4S)-2-(2-methoxy-5-methylphenyl)-4-(2-methoxyphenyl)tetrahydrofuran-2-carboxylic Acid

A mixture of Example 38B (73 mg, 0.205 mmol) and lithium hydroxide (34.3 mg, 1.434 mmol) in dioxane (1 mL) and water (0.3 mL) was stirred at 80° C. After 1 hour, the reaction mixture was reduced in volume and acidified with 2.0 M aqueous citric acid (1 mL). The aqueous layer was passed through an aqueous/organic separator tube with dichloromethane, and the organic layer was concentrated under reduced pressure to afford the title compound (65 mg, 0.190 mmol, 93% yield). ¹H NMR (400 MHz, CDCl₃) δ ppm 7.40 (dd, J=2.3, 0.7 Hz, 1H), 7.22 (ddd, J=8.2, 7.4, 1.7 Hz, 1H), 7.17 (dd, J=7.6, 1.7 Hz, 1H), 7.14-7.09 (m, 1H), 6.91 (dd, J=7.5, 1.2 Hz, 1H), 6.88-6.85 (m, 1H), 6.83 (d, J=8.3 Hz, 1H), 4.55-4.49 (m, 1H), 4.06-3.90 (m, 2H), 3.83 (s, 3H), 3.82 (s, 3H), 3.28-3.20 (m, 1H), 2.54-2.46 (m, 1H), 2.31 (d, J=0.7 Hz, 3H). MS(APCI+) m/z 343 (M+H)⁺.

### Example 38D

### (2R,4S)-2-(2-methoxy-5-methylphenyl)-4-(2-methoxyphenyl)-N-(2-methylquinoline-5-sulfonyl)oxolane-2-carboxamide

A solution of Example 38C (65 mg, 0.190 mmol), 1-(3-dimethylaminopropyl)-3-ethylcarbodiimide hydrochloride (72.8 mg, 0.380 mmol), and 4-dimethylaminopyridine (29.0 mg, 0.237 mmol) in anhydrous dichloromethane (1 mL) was stirred at ambient temperature for 30 minutes, and then 2-methylquinoline-5-sulfonamide (42.2 mg, 0.190 mmol) was added. The reaction was warmed to 38° C. and after 30 minutes became homogeneous. After 90 minutes, the reaction was quenched with 2.0 M aqueous citric acid (0.5 mL) and extracted with dichloromethane (2×10 mL) on an 8 mL Isolute phase separator. The solution was adsorbed onto silica gel and purified by flash chromatography (0-100% (3:1 ethyl acetate/ethanol) in dichloromethane, 10 g silica gel cartridge) to afford the title compound (73 mg, 0.134 mmol, 70.3% yield). ¹H NMR (400 MHz, dimethyl sulfoxide-d₆) δ ppm 12.04 (s, 1H), 8.82-8.72 (m, 1H), 8.26 (t, J=8.8 Hz, 2H), 7.90 (dd, J=8.4, 7.4 Hz, 1H), 7.46 (d, J=8.9 Hz, 1H), 7.25 (dd, J=2.3, 0.7 Hz, 1H), 7.16 (ddd, J=8.2, 7.3, 1.7 Hz, 1H), 7.04 (ddd, J=8.2, 3.0, 1.2 Hz, 2H), 6.91 (dd, J=8.3, 1.1 Hz, 1H), 6.82 (td, J=7.5, 1.1 Hz, 1H), 6.61 (d, J=8.3 Hz, 1H), 4.11-4.01 (m, 1H), 3.69 (s, 3H), 3.48-3.38 (m, 2H), 3.07 (s, 3H), 2.95 (dd, J=12.6, 7.3 Hz, 1H), 2.69 (s, 3H), 2.21 (s, 3H), 1.86 (dd, J=12.7, 11.0 Hz, 1H). MS(APCI+) m/z 547 (M+H)⁺.

### Example 39

### (2R,4R)-2-(2-methoxy-5-methylphenyl)-4-(2-methoxyphenyl)-N-(2-methylquinoline-5-sulfonyl)oxolane-2-carboxamide

### Example 39A

### (2R,4R)-methyl 2-(2-methoxy-5-methylphenyl)-4-(2-methoxyphenyl)tetrahydrofuran-2-carboxylate

To a solution of Example 38A (0.380 g, 1.020 mmol) and triethylsilane (0.326 mL, 2.041 mmol) in dichloromethane (4 mL) and cooled by an ice bath was added 2,2,2-trifluoroacetic acid (0.865 mL, 11.22 mmol), keeping the internal temperature below 3° C. The reaction was allowed to warm to ambient temperature and saturated aqueous sodium bicarbonate was added. The organic layer taken up in dichloromethane, passed through an aqueous/organic separator tube, and the organic layer was concentrated under reduced pressure. The crude material was purified by flash chromatography (0-30% tert-butyl methyl ether/hexanes, using a 40 g silica gel cartridge) to afford the title compound (213 mg, 0.598 mmol, 58.6% yield) as the first eluting isomer. ¹H NMR (400 MHz, CDCl₃) δ ppm 7.47 (d, J=2.3 Hz, 1H), 7.41 (dd, J=7.6, 1.7 Hz, 1H), 7.20 (ddd, J=8.1, 7.5, 1.7 Hz, 1H), 7.09 (ddd, J=8.2, 2.3, 0.9 Hz, 1H), 6.94 (td, J=7.5, 1.2 Hz, 1H), 6.84 (dd, J=8.2, 1.2 Hz, 1H), 6.77 (d, J=8.2 Hz, 1H), 4.52-4.46 (m, 1H), 3.89-3.80 (m, 1H), 3.79 (s, 3H), 3.75 (s, 3H), 3.74-3.70 (m, 1H), 3.69 (s, 3H), 3.27 (dd, J=12.8, 11.5 Hz, 1H), 2.41 (ddd, J=12.9, 7.7, 0.8 Hz, 1H), 2.36 (s, 3H). MS(APCI+) m/z 357 (M+H)⁺.

### Example 39B

### (2R,4R)-2-(2-methoxy-5-methylphenyl)-4-(2-methoxyphenyl)tetrahydrofuran-2-carboxylic Acid

A mixture of Example 39A (0.213 g, 0.598 mmol) and lithium hydroxide (0.100 g, 4.18 mmol) in dioxane (1.5 mL) and water (0.500 mL) was stirred at 80° C. After 2 hours, the reaction mixture was reduced in volume and acidified with 2.0 M aqueous citric acid (2 mL). The reaction was passed through an aqueous/organic separator tube with dichloromethane. The organic layer was concentrated to afford the title compound (0.188 g, 0.549 mmol, 92% yield). ¹H NMR (600 MHz, CDCl₃) δ ppm 9.74 (s, 1H), 7.43 (dd, J=2.2, 0.8 Hz, 1H), 7.31 (ddd, J=7.5, 1.7, 0.6 Hz, 1H), 7.25 (ddd, J=8.2, 7.4, 1.7 Hz, 1H), 7.14 (ddq, J=8.3, 2.3, 0.8 Hz, 1H), 6.96 (td, J=7.5, 1.1 Hz, 1H), 6.89 (dd, J=8.2, 1.1 Hz, 1H), 6.85 (d, J=8.3 Hz, 1H), 4.48 (t, J=7.8 Hz, 1H), 4.00 (dd, J=10.1, 8.1 Hz, 1H), 3.90-3.84 (m, 1H), 3.84 (s, 3H), 3.83 (s, 3H), 3.72 (s, 3H), 2.95 (dd, J=13.2, 11.2 Hz, 1H), 2.84 (dd, J=13.2, 7.7 Hz, 1H). MS(APCI+) m/z 343 (M+H)⁺.

### Example 39C

### (2R,4R)-2-(2-methoxy-5-methylphenyl)-4-(2-methoxyphenyl)-N-(2-methylquinoline-5-sulfonyl)oxolane-2-carboxamide

A solution of Example 39B (86 mg, 0.251 mmol), 1-(3-dimethylaminopropyl)-3-ethylcarbodiimide hydrochloride (96 mg, 0.502 mmol), and 4-dimethylaminopyridine (38.4 mg, 0.314 mmol) in anhydrous dichloromethane (1 mL) was stirred at ambient temperature for 30 minutes, and then 2-methylquinoline-5-sulfonamide (55.8 mg, 0.251 mmol) was added. The reaction was warmed to 38° C. and after 30 minutes, became homogeneous. After 90 minutes, the reaction was quenched with 2.0 M aqueous citric acid (0.5 mL) and extracted with dichloromethane (2×10 mL) on an 8 mL Isolute phase separator. The solution was adsorbed onto silica gel and purified by flash chromatography (0-100% (3:1 ethyl acetate/ethanol) in hexanes and then with 0-100% 3:1 (ethyl acetate/ethanol) in dichloromethane, 10 g silica gel cartridge) to afford the title compound (99 mg, 0.181 mmol, 72.1% yield). ¹H NMR (400 MHz, dimethyl sulfoxide-d₆) δ ppm 8.86 (d, J=8.9 Hz, 1H), 8.24 (dd, J=7.5, 1.3 Hz, 1H), 8.19 (d, J=8.4 Hz, 1H), 7.83 (dd, J=8.4, 7.4 Hz, 1H), 7.42 (d, J=8.9 Hz, 1H), 7.24 (d, J=2.3 Hz, 1H), 7.16 (td, J=7.8, 1.7 Hz, 1H), 7.05-6.95 (m, 2H), 6.90 (dd, J=8.3, 1.1 Hz, 1H), 6.80 (td, J=7.4, 1.2 Hz, 1H), 6.65 (d, J=8.3 Hz, 1H), 4.25-4.13 (m, 1H), 3.70 (s, 3H), 3.64-3.51 (m, 2H), 3.19 (s, 3H), 2.67 (s, 3H), 2.64-2.54 (m, 1H), 2.38-2.29 (m, 1H), 2.24 (s, 3H). MS(APCI+) m/z 547 (M+H)⁺.

### Example 40

### (2R,4R)—N-(2-aminoquinoline-5-sulfonyl)-2-(2-methoxy-5-methylphenyl)-4-(2-methoxyphenyl)oxolane-2-carboxamide

### Example 40A

### (2R,4R)—N-((2-chloroquinolin-5-yl)sulfonyl)-2-(2-methoxy-5-methylphenyl)-4-(2-methoxyphenyl)tetrahydrofuran-2-carboxamide

A solution of Example 39B (96 mg, 0.280 mmol), 1-(3-dimethylaminopropyl)-3-ethylcarbodiimide hydrochloride (108 mg, 0.561 mmol), and 4-dimethylaminopyridine (42.8 mg, 0.350 mmol) in anhydrous dichloromethane (1 mL) was stirred at ambient temperature for 30 minutes, and then 2-chloroquinoline-5-sulfonamide (68.0 mg, 0.280 mmol) was added. The reaction was warmed to 38° C. and after 30 minutes became homogeneous. After 90 minutes, the reaction was quenched with 2.0 M aqueous citric acid (0.5 mL) and extracted with dichloromethane (2×10 mL) on an 8 mL Isolute phase separator. The solution was adsorbed onto silica gel and purified by flash chromatography (0-100% ethyl acetate in dichloromethane, 10 g silica gel cartridge) to afford the title compound (84 mg, 0.148 mmol, 52.8% yield). ¹H NMR (500 MHz, dimethyl sulfoxide-d₆) δ ppm 12.12 (s, 1H), 8.99 (dd, J=9.1, 0.8 Hz, 1H), 8.38 (dd, J=7.5, 1.2 Hz, 1H), 8.29 (dt, J=8.4, 1.1 Hz, 1H), 8.01 (dd, J=8.5, 7.4 Hz, 1H), 7.70 (d, J=9.1 Hz, 1H), 7.25 (dd, J=2.2, 0.8 Hz, 1H), 7.19 (ddd, J=8.2, 7.4, 1.7 Hz, 1H), 7.05 (ddd, J=8.2, 2.3, 0.8 Hz, 1H), 6.99 (dd, J=7.7, 1.7 Hz, 1H), 6.92 (dd, J=8.3, 1.2 Hz, 1H), 6.81 (td, J=7.5, 1.1 Hz, 1H), 6.63 (d, J=8.3 Hz, 1H), 4.27-4.17 (m, 1H), 3.70 (s, 3H), 3.61-3.47 (m, 2H), 3.09 (s, 3H), 2.63-2.55 (m, 1H), 2.32-2.19 (m, 4H). MS(APCI+) m/z 567 (M+H)⁺.

### Example 40B

### (2R,4R)—N-(2-aminoquinoline-5-sulfonyl)-2-(2-methoxy-5-methylphenyl)-4-(2-methoxyphenyl)oxolane-2-carboxamide

A mixture Example 40A (84 mg, 0.148 mmol) and Pd SPHOS G4 (5.88 mg, 7.41 μmol) in dioxane (2 mL) was purged with N2 gas. Lithium bis(trimethylsilyl)amide (0.444 mL, 0.444 mmol) was added at ambient temperature, and the reaction heated to 80° C. for 1 hour. The reaction was cooled to ambient temperature and quenched with acetic acid (100 μL). The reaction mixture was concentrated under reduced pressure, and the crude material diluted with 1:1 dimethyl sulfoxide/methanol and purified by reverse-phase preparative HPLC (Phenomenex® Luna® C8(2) 5 μm 100 Å AXIA™ column (30 mm×150 mm), gradient of acetonitrile (A) and 0.1% trifluoroacetic acid in water (B), 50 mL/minute (0-0.5 minutes 10% A, 0.5-7.0 minutes linear gradient 10-95% A, 7.0-10.0 minutes 95% A, 10.0-12.0 minutes linear gradient 95-10% A)) to afford the desired product (68 mg). The product was triturated with methanol and ethyl acetate, and the soluble portion treated with decolorizing charcoal and filtered through diatomaceous earth. The solvent was removed under reduced pressure to afford the title compound (24 mg, 0.044 mmol, 29.6% yield). ¹H NMR (400 MHz, dimethyl sulfoxide-d₆) δ ppm 8.73 (dd, J=9.6, 0.8 Hz, 1H), 7.97 (dd, J=7.4, 1.3 Hz, 1H), 7.82 (dt, J=8.4, 1.1 Hz, 1H), 7.73 (dd, J=8.4, 7.5 Hz, 1H), 7.25 (d, J=2.3 Hz, 1H), 7.15 (ddd, J=8.2, 7.4, 1.7 Hz, 1H), 7.07-7.00 (m, 2H), 6.96 (d, J=9.6 Hz, 1H), 6.90 (dd, J=8.2, 1.1 Hz, 1H), 6.81 (td, J=7.5, 1.2 Hz, 1H), 6.69 (d, J=8.3 Hz, 1H), 4.26-4.15 (m, 1H), 3.70 (s, 3H), 3.62-3.56 (m, 2H), 3.30 (s, 3H), 3.17 (s, 2H), 2.70-2.58 (m, 1H), 2.34 (dd, J=13.0, 7.1 Hz, 1H), 2.25 (s, 3H). MS(APCI+) m/z 548 (M+H)⁺.

### Example 41

### (2R,4R)—N-(2-aminoquinoline-5-sulfonyl)-2-(2-methoxy-5-methylphenyl)-4-phenyloxolane-2-carboxamide

### Example 41A

### (2R,4R)-methyl 2-(2-methoxy-5-methylphenyl)-4-phenyltetrahydrofuran-2-carboxylate

To a solution of Example 32A (0.296 g, 0.865 mmol) and triethylsilane (0.276 mL, 1.729 mmol) in dichloromethane (4 mL) at about −7° C. was added 2,2,2-trifluoroacetic acid (0.733 mL, 9.51 mmol), maintaining the internal temperature at −7° C. The reaction was allowed to warm to ambient temperature. After about an hour, the reaction was treated carefully with saturated aqueous sodium bicarbonate. The organic layer taken up in dichloromethane, passed through an aqueous/organic separator tube, and concentrated under reduced pressure. The crude material was purified by flash chromatography (0-30% tert-butyl methyl ether/hexanes, 40 g silica gel cartridge) to afford the title compound (0.137 g, 0.420 mmol, 48.6% yield) as the first eluting isomer. ¹H NMR (400 MHz, CDCl₃) δ ppm 7.44 (d, J=2.2 Hz, 1H), 7.35-7.27 (m, 4H), 7.25-7.18 (m, 1H), 7.10 (ddd, J=8.2, 2.4, 0.8 Hz, 1H), 6.78 (d, J=8.2 Hz, 1H), 4.49-4.41 (m, 1H), 3.93 (dd, J=10.8, 8.3 Hz, 1H), 3.75 (s, 3H), 3.71 (s, 3H), 3.38 (tt, J=10.7, 7.4 Hz, 1H), 3.26 (dd, J=12.7, 11.6 Hz, 1H), 2.48-2.41 (m, 1H), 2.36 (d, J=0.7 Hz, 3H). MS(APCI+) m/z 327 (M+H)⁺.

### Example 41B

### (2R,4R)-2-(2-methoxy-5-methylphenyl)-4-phenyltetrahydrofuran-2-carboxylic Acid

A mixture of Example 41A (0.120 g, 0.368 mmol) and lithium hydroxide (78 mg, 3.26 mmol) in dioxane (1.5 mL) and water (0.500 mL) was stirred at 80° C. After 2 hours, the reaction mixture was reduced in volume and acidified with 2.0 M aqueous citric acid (1.5 mL). The reaction was passed through an aqueous/organic separator tube with dichloromethane. The organic layer was concentrated to afford the title compound (0.110 g, 0.352 mmol, 96% yield). ¹H NMR (500 MHz, CDCl₃) δ ppm 7.42 (dd, J=2.2, 0.8 Hz, 1H), 7.34-7.28 (m, 4H), 7.25-7.21 (m, 1H), 7.12 (ddd, J=8.2, 2.2, 0.8 Hz, 1H), 6.81 (d, J=8.3 Hz, 1H), 4.46 (td, J=7.8, 7.3, 0.8 Hz, 1H), 3.98 (dd, J=10.6, 8.4 Hz, 1H), 3.79 (s, 3H), 3.47 (tt, J=10.9, 7.7 Hz, 1H), 3.04 (dd, J=13.3, 11.4 Hz, 1H), 2.70 (ddd, J=13.3, 7.8, 0.8 Hz, 1H), 2.35 (d, J=0.7 Hz, 3H). MS(APCI+) m/z 313 (M+H)⁺.

### Example 41C

### (2R,4R)—N-((2-chloroquinolin-5-yl)sulfonyl)-2-(2-methoxy-5-methylphenyl)-4-phenyltetrahydrofuran-2-carboxamide

A solution of Example 41B (98 mg, 0.314 mmol), 1-(3-dimethylaminopropyl)-3-ethylcarbodiimide hydrochloride (120 mg, 0.627 mmol), and 4-dimethylaminopyridine (47.9 mg, 0.392 mmol) in anhydrous dichloromethane (1 mL) was stirred at ambient temperature for 30 minutes. 2-Chloroquinoline-5-sulfonamide (76 mg, 0.314 mmol) was added, and the reaction was warmed to 38° C. and after 30 minutes became homogeneous. After 90 minutes, the reaction was quenched with 2.0 M aqueous citric acid (0.5 mL) and extracted with dichloromethane (2×10 mL) on an 8 mL Isolute phase separator. The solution was adsorbed onto silica gel and purified by flash chromatography (0-100% ethyl acetate in dichloromethane, 10 g silica gel cartridge) to afford the title compound (0.133 g, 0.248 mmol, 79% yield). ¹H NMR (400 MHz, dimethyl sulfoxide-d₆) δ ppm 12.17 (s, 1H), 9.04-8.94 (m, 1H), 8.39 (dd, J=7.4, 1.3 Hz, 1H), 8.29 (dt, J=8.5, 1.1 Hz, 1H), 8.02 (dd, J=8.5, 7.4 Hz, 1H), 7.71 (d, J=9.1 Hz, 1H), 7.30 (dd, J=2.3, 0.7 Hz, 1H), 7.28-7.16 (m, 3H), 7.14-7.02 (m, 3H), 6.64 (d, J=8.3 Hz, 1H), 4.26 (t, J=7.9 Hz, 1H), 3.48 (dd, J=10.3, 8.3 Hz, 1H), 3.32-3.23 (m, 1H), 3.10 (s, 3H), 2.62-2.52 (m, 1H), 2.36-2.23 (m, 4H). MS(APCI+) m/z 537 (M+H)⁺.

### Example 41D

### (2R,4R)—N-(2-aminoquinoline-5-sulfonyl)-2-(2-methoxy-5-methylphenyl)-4-phenyloxolane-2-carboxamide

A mixture of Example 41C (133 mg, 0.248 mmol) and Pd SPHOS G4 (9.84 mg, 0.012 mmol) in dioxane (3 mL) was purged with N2 gas. Lithium bis(trimethylsilyl)amide (0.743 mL, 0.743 mmol) was added at ambient temperature, and the reaction heated to 80° C. for 1 hour. The reaction was cooled to ambient temperature and quenched with acetic acid (100 μL). The reaction mixture was concentrated under reduced pressure. The residue was diluted with 1:1 dimethyl sulfoxide/methanol and purified by reverse-phase preparative HPLC (Phenomenex® Luna® C8(2) 5 μm 100 Å AXIA™ column (30 mm×150 mm); gradient of acetonitrile (A) and 0.1% trifluoroacetic acid in water (B), 50 mL/minute (0-0.5 minutes 10% A, 0.5-7.0 minutes linear gradient 10-95% A, 7.0-10.0 minutes 95% A, 10.0-12.0 minutes linear gradient 95-10% A)) to afford the desired product (42 mg). The product was dissolved in ethyl acetate and treated with decolorizing charcoal, filtered through diatomaceous earth, and the solvent removed under reduced pressure to afford the title compound as a trifluoroacetic acid salt (42 mg, 0.066 mmol, 26.8% yield). ¹H NMR (600 MHz, dimethyl sulfoxide-d₆) δ ppm 12.01 (s, 1H), 8.78 (d, J=9.7 Hz, 1H), 8.49 (s, 2H), 8.07 (dd, J=7.1, 1.6 Hz, 1H), 7.95-7.86 (m, 2H), 7.32 (d, J=2.3 Hz, 1H), 7.28-7.23 (m, 2H), 7.22-7.18 (m, 1H), 7.16-7.12 (m, 2H), 7.08 (dd, J=8.3, 2.2 Hz, 1H), 7.03 (d, J=9.7 Hz, 1H), 6.71 (d, J=8.3 Hz, 1H), 4.29 (t, J=8.0 Hz, 1H), 3.52 (dd, J=10.3, 8.4 Hz, 1H), 3.35-3.31 (m, 1H), 3.26 (s, 3H), 2.62 (dd, J=13.2, 10.6 Hz, 1H), 2.32 (dd, J=13.2, 8.2 Hz, 1H), 2.29 (s, 3H). MS(APCI+) m/z 518 (M+H)⁺.

### Example 42

### (2R,4R)-2-(2-methoxy-5-methylphenyl)-4-(3-methoxyphenyl)-N-(2-methylquinoline-5-sulfonyl)oxolane-2-carboxamide

### Example 42A

### (2R,4R)-methyl 4-hydroxy-2-(2-methoxy-5-methylphenyl)-4-(3-methoxyphenyl)tetrahydrofuran-2-carboxylate

To a solution of Example 28A (0.270 g, 1.022 mmol) and lanthanum(III) chloride bis(lithium chloride) complex (1.703 mL, 1.022 mmol, 0.6 M in tetrahydrofuran) in tetrahydrofuran (5.11 mL) at −78° C. was added (3-methoxyphenyl)magnesium bromide (1.328 mL, 1.328 mmol, 1 M in tetrahydrofuran) dropwise and the reaction was allowed to warm to 0° C. over 2 hours. The reaction was diluted with dichloromethane, quenched with saturated aqueous ammonium chloride (2 mL), diluted with water, and extracted with dichloromethane (3×25 mL). The combined organic layers were concentrated, and the crude product was purified by flash chromatography (0-50% tert-butyl methyl ether/hexanes, 20 g silica gel cartridge) to afford the title compound (0.268 g, 0.720 mmol, 70.4% yield). ¹H NMR (400 MHz, CDCl₃) δ ppm 7.45 (d, J=2.3 Hz, 1H), 7.24 (t, J=8.0 Hz, 1H), 7.16-7.09 (m, 2H), 7.02 (ddd, J=7.7, 1.7, 0.9 Hz, 1H), 6.86-6.74 (m, 2H), 4.90 (s, 1H), 4.30 (dd, J=9.1, 1.6 Hz, 1H), 4.26 (d, J=9.1 Hz, 1H), 3.82 (s, 3H), 3.75 (s, 3H), 3.74 (s, 3H), 3.44 (dd, J=14.8, 1.6 Hz, 1H), 2.44 (d, J=14.7 Hz, 1H), 2.36 (s, 3H). MS(APCI+) m/z 355 (M−OH)⁺.

### Example 42B

### (2R,4R)-methyl 2-(2-methoxy-5-methylphenyl)-4-(3-methoxyphenyl)tetrahydrofuran-2-carboxylate

To a solution of Example 42A (0.268 g, 0.720 mmol) and triethylsilane (0.230 mL, 1.439 mmol) in dichloromethane (4 mL) and cooled in an ice/acetone bath was added 2,2,2-trifluoroacetic acid (0.610 mL, 7.92 mmol), keeping the internal temperature below 3° C. The reaction was allowed to warm to ambient temperature and stirred for 15 hours. The reaction mixture was concentrated under reduced pressure, and the residue was purified by flash chromatography (10-30% tert-butyl methyl ether/hexanes, 20 g silica gel cartridge) to afford a 3:1 mixture of diastereomers. The diastereomers were separated by chiral preparative supercritical fluid chromatography (ChiralPak AD-H column (30×250 mm, 5 micron), 15 mg/mL in methanol, 110 g/minutes CO₂, RT 2.45 minutes) to afford the title compound (115 mg, 0.323 mmol, 44.8% yield). ¹H NMR (400 MHz, CDCl₃) δ ppm 7.43 (d, J=2.3 Hz, 1H), 7.22 (t, J=7.9 Hz, 1H), 7.10 (ddd, J=8.1, 2.4, 0.8 Hz, 1H), 6.94-6.90 (m, 1H), 6.87 (t, J=2.1 Hz, 1H), 6.81-6.74 (m, 2H), 4.45 (td, J=7.6, 7.1, 0.9 Hz, 1H), 3.93 (dd, J=10.7, 8.2 Hz, 1H), 3.80 (s, 3H), 3.75 (s, 3H), 3.70 (s, 3H), 3.35 (tt, J=10.7, 7.3 Hz, 1H), 3.25 (dd, J=12.6, 11.6 Hz, 1H), 2.49-2.40 (m, 1H), 2.36 (s, 3H). MS(APCI+) m/z 357 (M+H)⁺.

### Example 42C

### (2R,4R)-2-(2-methoxy-5-methylphenyl)-4-(3-methoxyphenyl)tetrahydrofuran-2-carboxylic Acid

A mixture of Example 42B (125 mg, 0.351 mmol) and lithium hydroxide (50.4 mg, 2.104 mmol) in dioxane (1.5 mL) and water (0.500 mL) was stirred at 80° C. After 2 hours, the reaction was cooled, reduced in volume, and acidified with 2.0 M aqueous citric acid (2 mL). The aqueous layer was passed through an aqueous/organic separator tube with dichloromethane. The organic layer was concentrated to afford the title compound (0.115 g, 0.336 mmol, 96% yield). ¹H NMR (500 MHz, CDCl₃) δ ppm 7.40 (d, J=2.2 Hz, 1H), 7.25-7.21 (m, 1H), 7.14-7.09 (m, 1H), 6.90-6.87 (m, 1H), 6.83 (t, J=2.1 Hz, 1H), 6.82-6.80 (m, 1H), 6.78 (ddd, J=8.3, 2.7, 0.9 Hz, 1H), 4.42 (t, J=7.9 Hz, 1H), 3.96 (dd, J=10.4, 8.4 Hz, 1H), 3.80 (s, 3H), 3.79 (s, 3H), 3.46 (tt, J=10.7, 7.7 Hz, 1H), 2.98 (dd, J=13.3, 11.2 Hz, 1H), 2.76 (dd, J=13.3, 7.8 Hz, 1H), 2.34 (s, 3H). MS(APCI+) m/z 343 (M+H)⁺.

### Example 42D

### (2R,4R)-2-(2-methoxy-5-methylphenyl)-4-(3-methoxyphenyl)-N-(2-methylquinoline-5-sulfonyl)oxolane-2-carboxamide

A solution of Example 42C (0.115 g, 0.336 mmol), 1-(3-dimethylaminopropyl)-3-ethylcarbodiimide hydrochloride (0.129 g, 0.672 mmol), and 4-dimethylaminopyridine (0.051 g, 0.420 mmol) in anhydrous dichloromethane (2 mL) was stirred at ambient temperature for 30 minutes. 2-Methylquinoline-5-sulfonamide (78 mg, 0.351 mmol) was added, and the reaction was stirred at ambient temperature for 16 hours. The reaction was quenched with 2.0 M aqueous citric acid (1.0 mL) and extracted with dichloromethane (4×4 mL) on an 8 mL Isolute phase separator. The organic layer was concentrated under reduced pressure. The residue was triturated with methanol and filtered to afford the title compound (138 mg, 0.252 mmol, 75% yield). ¹H NMR (400 MHz, dimethyl sulfoxide-d₆) δ ppm 11.44 (s, 1H), 8.83 (dd, J=8.9, 0.9 Hz, 1H), 8.24 (dd, J=7.5, 1.2 Hz, 1H), 8.18 (dt, J=8.4, 1.1 Hz, 1H), 7.82 (dd, J=8.4, 7.4 Hz, 1H), 7.40 (d, J=8.9 Hz, 1H), 7.26 (d, J=2.2 Hz, 1H), 7.10 (t, J=7.9 Hz, 1H), 7.02 (ddd, J=8.2, 2.3, 0.9 Hz, 1H), 6.73 (ddd, J=8.2, 2.6, 0.9 Hz, 1H), 6.69 (t, J=2.2 Hz, 1H), 6.66-6.61 (m, 2H), 4.21 (t, J=8.0 Hz, 1H), 3.69 (s, 3H), 3.52 (dd, J=9.9, 8.4 Hz, 1H), 3.30 (tt, J=9.8, 7.9 Hz, 1H), 3.16 (s, 3H), 2.66 (s, 3H), 2.58 (dd, J=13.2, 10.2 Hz, 1H), 2.34 (dd, J=13.2, 8.4 Hz, 1H), 2.24 (s, 3H). MS(APCI+) m/z 547 (M+H)⁺.

### Example 43

### (2R,4S)-2-(2-methoxy-5-methylphenyl)-4-(3-methoxyphenyl)-N-(2-methylquinoline-5-sulfonyl)oxolane-2-carboxamide

### Example 43A

### (2R,4S)-methyl 2-(2-methoxy-5-methylphenyl)-4-(3-methoxyphenyl)tetrahydrofuran-2-carboxylate

To a solution of Example 42A (0.268 g, 0.720 mmol) and triethylsilane (0.230 mL, 1.439 mmol) in dichloromethane (4 mL) and cooled by an ice/acetone bath was added 2,2,2-trifluoroacetic acid (0.610 mL, 7.92 mmol), keeping the internal temperature below 3° C. The reaction was allowed to warm to ambient temperature, stirred for 15 hours, and concentrated under reduced pressure. The residue was purified by flash chromatography (10-30% tert-butyl methyl ether/hexanes, 20 g silica gel cartridge) to afford a 3:1 mixture of diastereomers. The diastereomers were separated by chiral preparative supercritical fluid chromatography (ChiralPak AD-H column (30×250 mm, 5 micron), 15 mg/mL in methanol, 110 g/minutes CO₂, RT 2.95 minutes) to afford the title compound (32 mg, 0.090 mmol, 12.48% yield). ¹H NMR (400 MHz, CDCl₃) δ ppm 7.48 (d, J=2.2 Hz, 1H), 7.19 (td, J=7.7, 0.7 Hz, 1H), 7.11-7.03 (m, 1H), 6.82-6.78 (m, 1H), 6.78-6.71 (m, 3H), 4.43 (t, J=7.8 Hz, 1H), 3.97 (dd, J=9.4, 8.1 Hz, 1H), 3.84-3.77 (m, 1H), 3.76 (s, 3H), 3.73 (s, 3H), 3.69 (s, 3H), 3.54-3.46 (m, 1H), 2.33 (d, J=0.7 Hz, 3H), 1.98 (dd, J=13.1, 10.6 Hz, 1H). MS(APCI+) m/z 357 (M+H)⁺.

### Example 43B

### (2R,4S)-2-(2-methoxy-5-methylphenyl)-4-(3-methoxyphenyl)tetrahydrofuran-2-carboxylic Acid

A mixture of Example 43A (32 mg, 0.090 mmol) and lithium hydroxide (36 mg, 1.503 mmol) in dioxane (1.5 mL) and water (0.500 mL) was stirred at 80° C. After 2 hours, the reaction mixture was reduced in volume and acidified with 2.0 M aqueous citric acid (1 mL). The aqueous layer was passed through an aqueous/organic separator tube with dichloromethane. The organic layer was concentrated under reduced pressure to afford the title compound (28 mg, 0.082 mmol, 91% yield). ¹H NMR (600 MHz, CDCl₃) δ ppm 7.44-7.42 (m, 1H), 7.23-7.19 (m, 1H), 7.12 (ddd, J=8.2, 2.3, 0.8 Hz, 1H), 6.84-6.80 (m, 2H), 6.78-6.74 (m, 2H), 4.48 (t, J=8.0 Hz, 1H), 3.99 (dd, J=9.7, 8.3 Hz, 1H), 3.81 (s, 3H), 3.77 (s, 3H), 3.74-3.67 (m, 1H), 3.37 (dd, J=13.0, 7.3 Hz, 1H), 2.36-2.28 (m, 4H). MS(APCI+) m/z 343 (M+H)⁺.

### Example 43C

### (2R,4S)-2-(2-methoxy-5-methylphenyl)-4-(3-methoxyphenyl)-N-(2-methylquinoline-5-sulfonyl)oxolane-2-carboxamide

A solution of Example 43B (28 mg, 0.085 mmol), 1-(3-dimethylaminopropyl)-3-ethylcarbodiimide hydrochloride (32.5 mg, 0.169 mmol), and 4-dimethylaminopyridine (12.93 mg, 0.106 mmol) in anhydrous dichloromethane (0.5 mL) was stirred at ambient temperature for 30 minutes. 2-Methylquinoline-5-sulfonamide (21 mg, 0.094 mmol) was added, and the reaction was stirred at ambient temperature for 16 hours. The reaction was quenched with 2.0 M citric acid (1.0 mL), extracted with dichloromethane (4×4 mL) on an 8 mL Isolute phase separator. and the organic layer was concentrated under reduced pressure. The residue was triturated with methanol and filtered to afford the title compound (35 mg, 0.064 mmol, 76% yield). ¹H NMR (400 MHz, dimethyl sulfoxide-d₆) δ ppm 11.44 (s, 1H), 8.82 (dd, J=8.9, 0.9 Hz, 1H), 8.25 (dd, J=7.4, 1.2 Hz, 1H), 8.20 (dt, J=8.4, 1.1 Hz, 1H), 7.84 (dd, J=8.4, 7.4 Hz, 1H), 7.43 (d, J=8.9 Hz, 1H), 7.26 (d, J=2.2 Hz, 1H), 7.13 (t, J=7.9 Hz, 1H), 7.01 (ddd, J=8.2, 2.3, 0.8 Hz, 1H), 6.72 (ddd, J=8.3, 2.6, 1.0 Hz, 1H), 6.65-6.58 (m, 3H), 4.11 (t, J=7.9 Hz, 1H), 3.75 (t, J=8.5 Hz, 1H), 3.68 (s, 3H), 3.22 (s, 3H), 3.20-3.11 (m, 1H), 3.06 (dd, J=12.8, 7.6 Hz, 1H), 2.68 (s, 3H), 2.20 (s, 3H), 1.90 (dd, J=12.8, 10.1 Hz, 1H). MS(APCI+) m/z 547 (M+H)⁺.

### Example 44

### (2R,4R)-2-(2-methoxy-5-methylphenyl)-4-(4-methoxyphenyl)-N-(2-methylquinoline-5-sulfonyl)oxolane-2-carboxamide

### Example 44A

### (2R,4R)-methyl 4-hydroxy-2-(2-methoxy-5-methylphenyl)-4-(4-methoxyphenyl)tetrahydrofuran-2-carboxylate

To a solution of Example 28A (0.250 g, 0.946 mmol) and lanthanum(III) chloride bis(lithium chloride) complex (1.577 mL, 0.946 mmol, 0.6 M in tetrahydrofuran) in tetrahydrofuran (4.73 mL) at −68° C. was added (4-methoxyphenyl)magnesium bromide (2.460 mL, 1.230 mmol, 0.5 M in tetrahydrofuran) dropwise, and the reaction was allowed to warm to 0° C. over 3 hours. The reaction was diluted with dichloromethane, quenched with saturated aqueous ammonium chloride (2 mL), diluted with water, and extracted with dichloromethane (3×25 mL). The combined organic layers were concentrated, and the residue was purified by flash chromatography (0-50% tert-butyl methyl ether/hexanes, 20 g silica gel cartridge) to afford the title compound (0.327 g, 0.878 mmol, 93% yield). ¹H NMR (500 MHz, CDCl₃) δ ppm 7.47-7.45 (m, 1H), 7.44-7.39 (m, 2H), 7.13 (ddt, J=8.2, 2.3, 0.7 Hz, 1H), 6.90-6.85 (m, 2H), 6.80 (d, J=8.2 Hz, 1H), 4.86 (s, 1H), 4.31 (dd, J=9.0, 1.8 Hz, 1H), 4.24 (d, J=9.0 Hz, 1H), 3.81 (s, 3H), 3.76 (d, J=0.4 Hz, 6H), 3.44 (dd, J=14.7, 1.8 Hz, 1H), 2.47-2.41 (m, 1H), 2.37 (d, J=0.7 Hz, 3H). MS(APCI+) m/z 355 (M−OH)⁺.

### Example 44B

### (2R)-methyl 2-(2-methoxy-5-methylphenyl)-4-(4-methoxyphenyl)tetrahydrofuran-2-carboxylate

To a solution of Example 44A (0.327 g, 0.878 mmol) and triethylsilane (0.280 mL, 1.756 mmol) in dichloromethane (4 mL) and cooled in an ice bath was added 2,2,2-trifluoroacetic acid (0.744 mL, 9.66 mmol), keeping the internal temperature below 3° C. The reaction was allowed to warm to ambient temperature and stirred overnight. The reaction mixture was concentrated under reduced pressure, and the residue was purified by flash chromatography (0-50% tert-butyl methyl ether/hexanes, 40 g silica gel cartridge) to afford a 65:35 mixture of diastereomers of the title compound (0.142 g, 0.398 mmol, 45.4% yield). ¹H NMR (600 MHz, CDCl₃) δ ppm 7.49-7.42 (m, 1H), 7.25-7.22 (m, 1H), 7.15-7.11 (m, 1H), 7.11-7.05 (m, 1H), 6.87-6.83 (m, 1H), 6.83-6.79 (m, 1H), 6.78-6.74 (m, 1H), 4.44-4.39 (m, 1H), 3.94-3.85 (m, 1H), 3.80-3.76 (m, 3H), 3.75-3.72 (m, 3H), 3.71-3.67 (m, 3H), 3.47 (dd, J=13.0, 7.5 Hz, 0.35H), 3.33 (tt, J=11.1, 7.5 Hz, 0.65H), 3.21 (dd, J=12.9, 11.7 Hz, 0.65H), 2.44-2.39 (m, 0.65H), 2.37-2.33 (m, 3H), 1.94 (dd, J=13.1, 11.0 Hz, 0.35H). MS(APCI+) m/z 357 (M+H)⁺.

### Example 44C

### (2R)-2-(2-methoxy-5-methylphenyl)-4-(4-methoxyphenyl)tetrahydrofuran-2-carboxylic acid

A mixture of Example 44B (0.142 g, 0.398 mmol) and lithium hydroxide (73 mg, 3.05 mmol) in methanol (0.5 mL) and water (0.500 mL) was stirred at ambient temperature for 16 hours. The reaction was acidified with 2.0 M aqueous citric acid (2.0 mL) and passed through an aqueous/organic separator tube with dichloromethane. The organic layer was concentrted to afford a diastereomeric mixture of the title compound (0.130 g, 0.380 mmol, 95% yield). MS(APCI+) m/z 342 (M+H)⁺.

### Example 44D

### (2R,4R)-2-(2-methoxy-5-methylphenyl)-4-(4-methoxyphenyl)-N-(2-methylquinoline-5-sulfonyl)oxolane-2-carboxamide

A solution of Example 44C (110 mg, 0.321 mmol), 1-(3-dimethylaminopropyl)-3-ethylcarbodiimide hydrochloride (123 mg, 0.643 mmol), and 4-dimethylaminopyridine (49.1 mg, 0.402 mmol) in anhydrous dichloromethane (3 mL) was stirred at ambient temperature for 30 minutes. 2-Methylquinoline-5-sulfonamide (75 mg, 0.337 mmol) was added, and the reaction was stirred at 38° C. After 2 hours, the reaction was quenched with 2.0 M aqueous citric acid (1.0 mL) and extracted with dichloromethane (2×10 mL) on a 30 mL Isolute phase separator. The organic layer was concentrated under reduced pressure, and the residue was purified by flash chromatography (0-100% (1:3 ethanol/ethyl acetate) in dichloromethane, 20 g silica gel cartridge) to afford a mixture of diastereomers (140 mg). The diastereomers were separated by chiral preparative supercritical fluid chromatography (ChiralPak IC column (21×250 mm, 5 micron), 10 mg/mL in methanol, 45 g/minutes CO₂, RT 6.4 minutes) to afford the title compound (49 mg, 0.090 mmol, 27.9% yield). ¹H NMR (500 MHz, CDCl₃) δ ppm 8.72 (s, 1H), 8.53 (s, 1H), 8.49 (d, J=7.4 Hz, 1H), 8.33 (s, 1H), 7.82 (t, J=7.7 Hz, 1H), 7.34 (d, J=8.9 Hz, 1H), 7.30 (dd, J=2.2, 0.7 Hz, 1H), 7.08 (ddd, J=8.2, 2.3, 0.8 Hz, 1H), 7.05 (d, J=8.6 Hz, 2H), 6.83-6.75 (m, 2H), 6.52 (d, J=8.2 Hz, 1H), 4.27 (t, J=8.0 Hz, 1H), 3.77 (s, 3H), 3.65 (dd, J=10.5, 8.5 Hz, 1H), 3.32 (tt, J=10.7, 7.8 Hz, 1H), 3.10 (s, 3H), 2.86-2.69 (m, 4H), 2.39 (dd, J=13.5, 8.0 Hz, 1H), 2.33 (d, J=0.7 Hz, 3H). MS(APCI+) m/z 547 (M+H)⁺.

### Example 45

### (2R,4S)-2-(2-methoxy-5-methylphenyl)-4-(4-methoxyphenyl)-N-(2-methylquinoline-5-sulfonyl)oxolane-2-carboxamide

A solution of Example 44C (110 mg, 0.321 mmol), 1-(3-dimethylaminopropyl)-3-ethylcarbodiimide hydrochloride (123 mg, 0.643 mmol), and 4-dimethylaminopyridine (49.1 mg, 0.402 mmol) in anhydrous dichloromethane (3 mL) was stirred at ambient temperature for 30 minutes. 2-Methylquinoline-5-sulfonamide (75 mg, 0.337 mmol) was added, and the reaction was stirred at 38° C. After 2 hours, the reaction was quenched with 2.0 M aqueous citric acid (1.0 mL) and extracted with dichloromethane (2×10 mL) on a 30 mL Isolute phase separator. The organic layer was concentrated under reduced pressure, and the residue was purified by flash chromatography (0-100% of (1:3 ethanol/ethyl acetate) in dichloromethane, 20 g silica gel cartridge) to afford a mixture of diastereomers (140 mg). The diastereomers were separated by chiral preparative supercritical fluid chromatography (ChiralPak IC column (21×250 mm, 5 micron), 10 mg/mL in methanol, 45 g/minutes CO₂, RT 7.5 minutes) to afford the title compound (22 mg, 0.040 mmol, 12.53% yield). ¹H NMR (400 MHz, CDCl₃) δ ppm 8.73 (s, 1H), 8.47 (d, J=7.5 Hz, 1H), 8.33 (s, 1H), 7.81 (t, J=8.0 Hz, 1H), 7.32 (d, J=9.0 Hz, 1H), 7.24 (d, J=2.2 Hz, 1H), 7.07-7.01 (m, 1H), 6.98 (d, J=8.7 Hz, 2H), 6.81-6.75 (m, 2H), 6.52 (d, J=8.3 Hz, 1H), 4.23 (t, J=8.0 Hz, 1H), 3.83 (dd, J=9.6, 8.2 Hz, 1H), 3.75 (s, 3H), 3.33 (p, J=8.9, 8.4 Hz, 1H), 3.22 (d, J=3.1 Hz, 3H), 3.20-3.14 (m, 1H), 2.79 (s, 4H), 2.26 (s, 3H), 1.98 (dd, J=13.0, 11.1 Hz, 1H). MS(APCI+) m/z 547 (M+H)⁺.

### Determination of Biological Activity

### Trans-Epithelial Current Clamp (TECC) Assay Using Primary Human Bronchial Epithelial Cells

A cell-based assay was developed using the primary human bronchial epithelial cells (hBE cells) with F508del/F508del CFTR and other mutations.

### Human Bronchial Epithelial Cells

Primary human bronchial epithelial (hBE) cells from CFTR patients with homozygous F508del/F508del mutation were expanded from 1×10⁶ to 250×10⁶ cells (Neuberger, T et al., 2011, Methods Mol Biol 741:39-54). For this purpose, cells isolated from CF patients with the homozygous mutation, procured from the CF center tissue procurement and cell culture core at the Marsico Lung Institute at UNC (Randell), the Cystic Fibrosis translational research center at McGill University (University), and Rosalind Franklin University Medical School (RFUMS) were seeded onto 24 well Corning (Cat #3378) filter plates that were coated with 3T3 conditioned media and grown at an air-liquid interface for 35 days using an Ultroser® G supplemented differentiation media. All the primary human bronchial epithelial cells were collected in accordance with institutional review board approval protocols. Apical surface mucus was removed 72 hours before the experiment by incubating the apical surface of the cells for 30 minutes with 3 mM dithiothreitol (DTT) prepared in Dulbecco's phosphate buffered saline (DPBS) with Ca²⁺ and Mg²⁺. This was followed with aspiration of the mucus from the apical surface along with DPBS. The apical surface was re-washed with phosphate buffered saline (PBS) incubated for 30 minutes followed with aspiration. These hBE cells were then incubated with the desired concentrations of the test C2 corrector compounds along with co-corrector, 4-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]benzoic acid and potentiator, (5-{3-amino-5-[4-(trifluoromethoxy)benzene-1-sulfonyl]pyridin-2-yl}-1,3,4-oxadiazol-2-yl)methanol, for 18-24 hours at 37° C., 5% CO₂. The desired concentrations of the correctors and potentiator compounds were prepared from the 10 mM stocks in differentiation media and were always applied on the basolateral side of the epithelial cells. We also used an assay format where a fixed concentration of potentiator was added chronically along with the corrector compounds. This chronic treatment with the potentiator helped eliminate any interaction that might happen with CFTR modulators (correctors and potentiators) and thereby determined the true efficacy of the modulator combinations reflective of clinical relevance.

### TECC (Transepithelial Current Clamp) Assay

The assay uses a Transepithelial Current Clamp (TECC) (Vu, C B et al., 2017; J Med Chem 60:458-473) instrument that can measure the functionality of the mutated channel by measuring the equivalent CFTR current (I_{EQ}) generated by the polarized primary epithelial cells. We used a TECC-24 with a 24-channel electrode manifold allowing for the simultaneous measurement of the transepithelial voltage, V_{T}, and transepithelial resistance, R_{T}, under current clamp conditions, from 24 filters using a 24 well Costar filter plate. The design of the filters in the 24 well filter plates was exactly the same as the design of an individual Transwell filter used in the classical Ussing Chamber with a surface area of 0.33 cm². Each measured V_{T} values were corrected for the electrode offset potential measured using buffer alone in a separate plate, and each measured R_{T} values were then corrected for the combined solution series and empty filter resistances. The corrected V_{T} and R_{T} values were then used to calculate the equivalent current, I_{EQ} using Ohm's law (I_{EQ}=V_{T}/R_{T}). The area under the curve (AUC) for the time period between the forskolin peak I_{EQ} response and at the time of bumetanide addition was also calculated using a one-third trapezoid method, in addition to calculating the I_{EQ}. The assay was run in a 24-well format and all 24-wells were measured at the same time point giving a higher throughput for this assay. On the day of measuring the corrector activity on the TECC, the cells were switched into a bicarbonate and serum free F-12 Coon's medium and allowed to equilibrate for 30 minutes for hBE cells in a CO₂ free incubator. At the time of measurement, the apical and basolateral sides of the filter were bathed with the F-12 Coon's modification media (with 20 mM 4-(2-hydroxyethyl)-1-piperazineethanesulfonic acid (HEPES), pH 7.4 (using 1 M tris(hydroxymethyl)aminomethane (Tris)), and the measurements were made at 36.5° C. Current responses before and after the sequential addition of benzamil (apical 6 μM addition; for inhibiting epithelial ENaC channel), forskolin (apical and basolateral 10 μM addition; for activating the CFTR channel), and bumetanide (basolateral 20 μM addition; for inhibiting the Na:2Cl:K co-transporter, an indirect measure of inhibiting the chloride secretion driven by CFTR channel) were measured.

All plates contained negative controls (dimethyl sulfoxide, DMSO) that sets the null response; and positive controls 4-[(2R,4R)-4-({[1-(2,2-difluoro-1,3-benzodioxol-5-yl)cyclopropyl]carbonyl}amino)-7-(difluoromethoxy)-3,4-dihydro-2H-chromen-2-yl]benzoic acid (0.15 μM) coupled with the control potentiator (5-{3-amino-5-[4-(trifluoromethoxy)benzene-1-sulfonyl]pyridin-2-yl}-1,3,4-oxadiazol-2-yl)methanol (0.45 μM) sets the 100% response to measure the correction of the mutated CFTR channel. The maximum percent activity (Emax) was reported relative to the positive control value.

The % activity measured at each of the 6 test concentrations of the test compound was normalized to the on-plate positive control using the following formula:

% activity=[(test compound response−DMSO response)/(positive control response−DMSO response)]*100

The I_{EQ} and AUC at different test concentrations were fit and an EC₅₀ was calculated using the general sigmoidal curve with variable Hill slope equation included in the Prism v5 software.

TABLE 2
Human Bronchial Epithelial Cell TECC Assay Data

| Ex | EC₅₀ (μM) | Emax (% Dual) |
|---|---|---|
| 1 | 1.71 | 226 |
| 2 | 0.0825 | 318 |
| 3 | 0.0504 | 154 |
| 4 | 0.503 | 167 |
| 5 | 0.0078 | 242 |
| 6 | 0.0195 | 235 |
| 7 | 0.0117 | 283 |
| 8 | 0.0032 | 339 |
| 9 | 0.0337 | 256 |
| 10 | >0.3 | 129 |
| 11 | 0.0171 | 158 |
| 12 | 0.068 | 315 |
| 13 | >0.3 | 119 |
| 14 | 0.244 | 161 |
| 15 | 0.123 | 178 |
| 16 | >0.3 | 110 |
| 17 | 0.0115 | 229 |
| 18 | >0.8 | 152 |
| 19 | 0.033 | 253 |
| 20 | 0.0351 | 201 |
| 21 | 0.0118 | 199 |
| 22 | 0.589 | 200 |
| 23 | 0.02 | 241 |
| 24 | >0.8 | 167 |
| 25 | 0.0306 | 279 |
| 26 | 0.171 | 130 |
| 27 | 0.0046 | 212 |
| 28 | 1.1 | 182 |
| 29 | 0.164 | 230 |
| 30 | 0.331 | 197 |
| 31 | >5 | 117 |
| 32 | 0.0208 | 263 |
| 33 | 0.0201 | 264 |
| 34 | 0.0346 | 284 |
| 35 | 0.443 | 248 |
| 36 | 0.031 | 255 |
| 37 | 0.0383 | 305 |
| 38 | 0.0141 | 244 |
| 39 | 0.0261 | 239 |
| 40 | 0.0026 | 208 |
| 41 | 0.0194 | 274 |
| 42 | 0.0111 | 237 |
| 43 | 0.0599 | 216 |
| 44 | 0.0116 | 335 |
| 45 | 0.0363 | 229 |

### Cell Surface Expression-Horse Radish Peroxidase (CSE-HRP) Assay

A cellular assay for measuring the F508delCFTR cell surface expression after correction with test compounds either without or with a co-corrector (2 μM of 3-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid), was developed in human lung derived epithelial cell line (CFBE41o-) (Veit G et al, (2012) Mol Biol Cell. 23(21): 4188-4202). The development was achieved by expressing the F508delCFTR mutation along with a horseradish peroxidase (HRP) in the fourth exofacial loop, and then measuring the HRP activity using luminescence readout from these cells, CFBE41o-F508delCFTR-HIRP, that were incubated overnight with the test corrector compounds, either without or with the co-corrector. For this primary assay, the CFBE41o-F508delCFTR-HRP cells were plated in 384-well plates (Greiner Bio-one; Cat 781080) at 4,000 cells/well along with 0.5 μg/mL doxycycline to induce the F508delCFTR-HRP expression and further incubated at 37° C., 500 CO₂ for 68-72 hours. The test compounds were then added either without or with a co-corrector at the required concentrations and further incubated for 18-24 hours at 33° C. The highest concentration tested was 20 μM or 30 μM (GI-1 to GIII-36) with an 8-point concentration response curve using a 3-fold dilution in both the test compound without or with the co-corrector. Three replicate plates were run to determine one EC₅₀. All plates contained negative controls (dimethyl sulfoxide, DMSO) and positive control (2 μM or 3 μM (GI-1 to GIII-36) of 3-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid) as well as on-plate concentration response of the positive control. Post incubation, the plates were washed 5× times with Dulbecco's phosphate buffered saline (DPBS), followed by the addition of the HRP substrate, luminol (50 μL), and measuring the HRP activity using luminescence readout on EnVision® Multilabel Plate Reader (Perkin Elmer; product number 2104-0010). The raw counts from the experiment were analyzed using Accelrys® Assay Explorer v3.3.

Z′ greater than 0.5 was used as passing quality control criteria for the plates. The Z′ is defined as:

1−[3*SD_{Positive Control}+3*SD_{Negative Control}/Absolute(Mean_{Positive Control}−Mean_{Negative Control})]

wherein “SD” is standard deviation.

The % activity measured at each of the 8 test concentrations of the test compound added either without or with a co-corrector (2 μM or 3 μM (GI-1 to GIII-36) of 3-[(2R,4R)-4-({[1-(2,2-difluoro-1,3-benzodioxol-5-yl)cyclopropyl]carbonyl}amino)-7-methoxy-3,4-dihydro-2H-chromen-2-yl]benzoic acid) was normalized to the on-plate positive control using the following formulae:

% activity (Test compound without co-corrector)=[(test compound without co-corrector response−DMSO response)/(positive control response−DMSO response)]*100

% activity (Test compound with co-corrector)=[(test compound with co-corrector response−DMSO response)/(positive control response−DMSO response)]*100

The maximum % activity achieved for the test compound either without or with a co-corrector at any tested concentration is presented in Table 3 along with the respective EC₅₀'s calculated using the following general sigmoidal curve with variable Hill slope equation (described as Model 42 in the Accelrys® Assay Explorer v3.3 software):

y=(a−d)/(1+(x/c)\hat{ }B)+d

General sigmoidal curve with concentration, response, top, bottom, EC₅₀ and Hill slope. This model describes a sigmoidal curve with an adjustable baseline, a. The equation can be used to fit curves where response is either increasing or decreasing with respect to the independent variable, “x”.

“x” is a concentration of drug under test.

“y” is the response.

“a” is the maximum response, and “d” is the minimum response

“c” is the inflection point (EC₅₀) for the curve. That is, “y” is halfway between the lower and upper asymptotes when x=c.

“b” is the slope-factor or Hill coefficient. The sign of b is positive when the response increases with increasing dose and is negative when the response decreases with increasing dose (inhibition).

TABLE 3
Cell Surface Expression-Horse Radish Peroxidase Assay Data

| | | Maximum % | | Maximum % |
|---|---|---|---|---|
| | EC₅₀ (without | activity (without | EC₅₀ (with | activity (with |
| | co-corrector) | co-corrector) | co-corrector) | co-corrector) |
| Ex | (μM) | (%) | (μM) | (%) |
| 1 | 4.94 | 233 | 3.78 | 546 |
| 2 | 1.54 | 602 | 0.78 | 1120 |
| 3 | 7.18 | 320 | — | — |
| 5 | 2.27 | 1320 | 1.21 | 2250 |
| 6 | 1.36 | 712 | 1.00 | 1620 |
| 7 | 0.53 | 509 | 0.33 | 1020 |
| 8 | 0.59 | 702 | 0.44 | 1630 |
| 9 | 1.65 | 865 | 0.84 | 1560 |
| 10 | 3.93 | 303 | 2.70 | 711 |
| 11 | 1.11 | 542 | 1.19 | 1520 |
| 12 | 0.70 | 735 | 0.68 | 1830 |
| 13 | 6.49 | 122 | 4.99 | 399 |
| 14 | 3.49 | 334 | 2.54 | 871 |
| 15 | 1.51 | 307 | 1.01 | 708 |
| 16 | 4.80 | 147 | 2.55 | 444 |
| 17 | 2.66 | 931 | 1.56 | 1830 |
| 19 | 0.88 | 339 | 0.72 | 850 |
| 20 | 1.23 | 366 | 0.68 | 862 |
| 21 | 1.54 | 857 | 1.29 | 1610 |
| 22 | 1.95 | 235 | 1.14 | 673 |
| 23 | 1.07 | 726 | 0.48 | 1413 |
| 24 | 3.63 | 141 | 2.52 | 449 |
| 25 | 1.45 | 476 | 0.58 | 954 |
| 26 | 3.97 | 282 | 3.27 | 828 |
| 27 | 1.47 | 709 | 0.65 | 1400 |
| 28 | 4.11 | 180 | 3.47 | 513 |
| 29 | 1.86 | 213 | 1.21 | 587 |
| 30 | 2.63 | 301 | — | — |
| 31 | 5.16 | 100 | — | — |
| 33 | 2.30 | 756 | 1.63 | 1455 |
| 36 | 1.59 | 443 | 0.81 | 1070 |
| 37 | 3.63 | 217 | 3.21 | 573 |
| 38 | 0.98 | 679 | 0.33 | 1333 |
| 39 | 0.47 | 470 | 0.20 | 1038 |
| 40 | 0.44 | 705 | 0.16 | 1362 |
| 41 | 0.76 | 872 | 0.27 | 1570 |
| 42 | 1.12 | 751 | 0.57 | 1510 |
| 44 | 0.60 | 512 | 0.30 | 1126 |
| 45 | 0.61 | 386 | 0.44 | 911 |

The data provided in the present application demonstrate that the compounds of the invention demonstrate activity in vitro and may be useful in vivo in the treatment of cystic fibrosis.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, Formulations and/or methods of use of the invention, may be made without departing from the spirit and scope thereof. All publications, patents, and patent applications cited herein are hereby incorporated by reference in their entirety for all purposes.

We claim:

1. A compound, or pharmaceutically acceptable salt thereof, of formula (I) 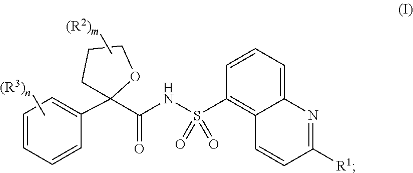
where
R¹ is selected from the group consisting of —NH₂, C₁-C₄ alkyl, and C₃-C₇ cycloalkyl;
R² is selected from the group consisting of C₁-C₄ alkyl, —OR^{2a}, and phenyl; wherein the R² phenyl is optionally substituted with one or more R⁴; wherein optionally two R² groups combine to form a C₃-C₆ cycloalkyl or 3-7 membered heterocyclyl;
R^{2a} is selected from the group consisting of C₁-C₄ alkyl, phenyl; wherein the phenyl is optionally substituted with one or more R⁵;
R³ is selected from the group consisting of C₁-C₄ alkyl and —OR^{3a};
R^{3a} is selected from the group consisting of C₁-C₄ alkyl, C₂-C₆ alkoxyalkyl, phenyl, and 5-6 membered heteroaryl; wherein the phenyl and 5-6 membered heteroaryl are optionally substituted with one or more R⁶;
R⁴ is —OR^{4a};
R^{4a} is C₁-C₄ alkyl;
R⁵ is —OR^{5a};
R^{5a} is C₁-C₄ alkyl;
R⁶ is —OR^{6a};
R^{6a} is selected from the group consisting of C₁-C₄ alkyl and C₁-C₄ haloalkyl;
m is 0, 1, 2, or 3; and
n is 0, 1, or 2.

2. The compound of claim 1, or pharmaceutically acceptable salt thereof, wherein R¹ is C₁-C₄ alkyl.

3. The compound of claim 2, or pharmaceutically acceptable salt thereof, wherein R^{3a} is C₁-C₄ alkyl; and n is 2.

4. The compound of claim 3, or pharmaceutically acceptable salt thereof, wherein m is 1 or 2.

5. The compound of claim 4, or pharmaceutically acceptable salt thereof, wherein R² is selected from the group consisting of C₁-C₄ alkyl and —OR^{2a}.

6. The compound of claim 4, or pharmaceutically acceptable salt thereof, wherein R² is phenyl optionally substituted with one or more R⁴.

7. The compound of claim 1, or a pharmaceutically acceptable salt thereof, of formula (II) 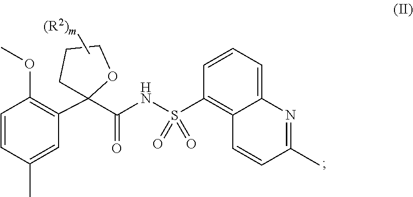
where
R² is selected from the group consisting of C₁-C₄ alkyl, —OR^{2a}, and phenyl; wherein the R² phenyl is optionally substituted with one or more R⁴; wherein optionally two R² groups combine to form a C₃-C₆ cycloalkyl or 3-7 membered heterocyclyl;
R^{2a} is selected from the group consisting of C₁-C₄ alkyl, phenyl; wherein the phenyl is optionally substituted with one or two R⁵;
R⁵ is —OR^{5a};
R^{5a} is C₁-C₄ alkyl; and
m is 0, 1, 2, or 3.

8. The compound of claim 7, or a pharmaceutically acceptable salt thereof, where m is 1; and R² is phenyl optionally substituted with one or more R⁴.

9. The compound of claim 1, or a pharmaceutically acceptable salt thereof, of formula (III) 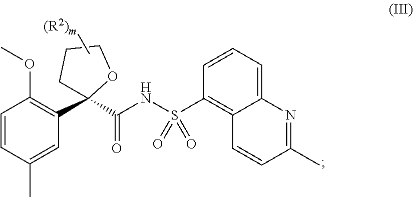
where
R² is selected from the group consisting of C₁-C₄ alkyl, —OR^{2a}, and phenyl; wherein the R² phenyl is optionally substituted with one or more R⁴; wherein optionally two R² groups combine to form a C₃-C₆ cycloalkyl or 3-7 membered heterocyclyl;
R^{2a} is selected from the group consisting of C₁-C₄ alkyl, phenyl; wherein the phenyl is optionally substituted with one or more R⁵;
R⁵ is —OR^{5a};
R^{5a} is C₁-C₄ alkyl; and
m is 0, 1, 2, or 3.

10. The compound of claim 9, or a pharmaceutically acceptable salt thereof, where m is 1; and R² is phenyl optionally substituted with one or more R⁴.

11. The compound of claim 1, or pharmaceutically acceptable salt thereof, which is

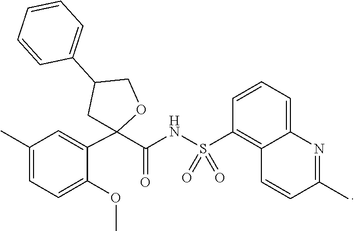

12. The compound of claim 2, which is

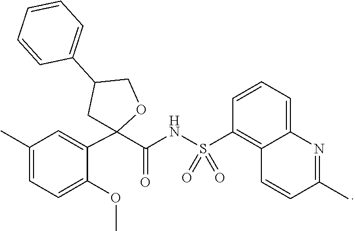

13. The compound of claim 1, or pharmaceutically acceptable salt thereof, which is

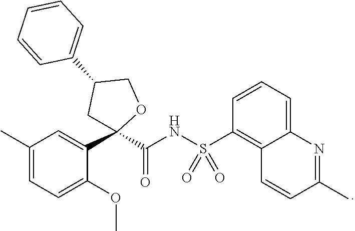

14. The compound of claim 1 which is

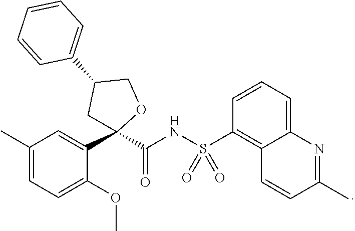

15. The compound of claim 1, or a pharmaceutically acceptable salt thereof, selected from the group consisting of
(2S)-2-(2-ethoxy-5-methylphenyl)-N-(2-methylquinoline-5-sulfonyl)oxolane-2-carboxamide;
(2R)-2-(2-ethoxy-5-methylphenyl)-N-(2-methylquinoline-5-sulfonyl)oxolane-2-carboxamide;
(2S)-2-(2-methoxy-5-methylphenyl)-4,4-dimethyl-N-(2-methylquinoline-5-sulfonyl)oxolane-2-carboxamide;
7-(2-methoxy-5-methylphenyl)-N-(2-methylquinoline-5-sulfonyl)-2,6-dioxaspiro[3.4]octane-7-carboxamide;
(2R)-2-(2-methoxy-5-methylphenyl)-4,4-dimethyl-N-(2-methylquinoline-5-sulfonyl)oxolane-2-carboxamide;
(2R)—N-(2-cyclopropylquinoline-5-sulfonyl)-2-(2-ethoxy-5-methylphenyl)oxolane-2-carboxamide;
(2S,5S)-2-(2-methoxy-5-methylphenyl)-N-(2-methylquinoline-5-sulfonyl)-5-phenyloxolane-2-carboxamide;
(2R,5S)-2-(2-methoxy-5-methylphenyl)-N-(2-methylquinoline-5-sulfonyl)-5-phenyloxolane-2-carboxamide;
(2R)-2-(2-ethoxy-5-ethylphenyl)-N-(2-methylquinoline-5-sulfonyl)oxolane-2-carboxamide;
(2S)-2-(2-ethoxy-5-ethylphenyl)-N-(2-methylquinoline-5-sulfonyl)oxolane-2-carboxamide;
(2S,5R)-2-(2-methoxy-5-methylphenyl)-N-(2-methylquinoline-5-sulfonyl)-5-phenyloxolane-2-carboxamide;
(2R,5R)-2-(2-methoxy-5-methylphenyl)-N-(2-methylquinoline-5-sulfonyl)-5-phenyloxolane-2-carboxamide;
(2S)-2-{5-ethyl-2-[(2S)-2-methoxypropoxy]phenyl}-N-(2-methylquinoline-5-sulfonyl)oxolane-2-carboxamide;
(2R)-2-{5-ethyl-2-[(2S)-2-methoxypropoxy]phenyl}-N-(2-methylquinoline-5-sulfonyl)oxolane-2-carboxamide;
(6R)-6-(2-methoxy-5-methylphenyl)-N-(2-methylquinoline-5-sulfonyl)-5-oxaspiro[2.4]heptane-6-carboxamide;
(6S)-6-(2-methoxy-5-methylphenyl)-N-(2-methylquinoline-5-sulfonyl)-5-oxaspiro[2.4]heptane-6-carboxamide;
2-(5-ethyl-2-phenoxyphenyl)-N-(2-methylquinoline-5-sulfonyl)oxolane-2-carboxamide;
2-{5-ethyl-2-[(2-methoxypyridin-3-yl)oxy]phenyl}-N-(2-methylquinoline-5-sulfonyl)oxolane-2-carboxamide;
(2R,4R)-2-(2-methoxy-5-methylphenyl)-4-methyl-N-(2-methylquinoline-5-sulfonyl)oxolane-2-carboxamide;
(2R,4S)-2-(2-methoxy-5-methylphenyl)-4-methyl-N-(2-methylquinoline-5-sulfonyl)oxolane-2-carboxamide;
2-(2-{[2-(difluoromethoxy)pyridin-3-yl]oxy}-5-ethylphenyl)-N-(2-methylquinoline-5-sulfonyl)oxolane-2-carboxamide;
(2S)-2-(5-ethyl-2-phenoxyphenyl)-N-(2-methylquinoline-5-sulfonyl)oxolane-2-carboxamide;
(2R)-2-(5-ethyl-2-phenoxyphenyl)-N-(2-methylquinoline-5-sulfonyl)oxolane-2-carboxamide;
(2S)-2-{5-ethyl-2-[(2-methoxypyridin-3-yl)oxy]phenyl}-N-(2-methylquinoline-5-sulfonyl)oxolane-2-carboxamide;
(2R)-2-{5-ethyl-2-[(2-methoxypyridin-3-yl)oxy]phenyl}-N-(2-methylquinoline-5-sulfonyl)oxolane-2-carboxamide;
(2S)-2-(2-{[2-(difluoromethoxy)pyridin-3-yl]oxy}-5-ethylphenyl)-N-(2-methylquinoline-5-sulfonyl)oxolane-2-carboxamide;
(2R)-2-(2-{[2-(difluoromethoxy)pyridin-3-yl]oxy}-5-ethylphenyl)-N-(2-methylquinoline-5-sulfonyl)oxolane-2-carboxamide;
(2R,4S)-4-methoxy-2-(2-methoxy-5-methylphenyl)-N-(2-methylquinoline-5-sulfonyl)oxolane-2-carboxamide;
(2R,4R)-4-methoxy-2-(2-methoxy-5-methylphenyl)-N-(2-methylquinoline-5-sulfonyl)oxolane-2-carboxamide;
(2R,4S)-4-methoxy-2-(2-methoxy-5-methylphenyl)-4-methyl-N-(2-methylquinoline-5-sulfonyl)oxolane-2-carboxamide;
(2S,4R)-4-methoxy-2-(2-methoxy-5-methylphenyl)-4-methyl-N-(2-methylquinoline-5-sulfonyl)oxolane-2-carboxamide;
(2R,4S)-2-(2-methoxy-5-methylphenyl)-N-(2-methylquinoline-5-sulfonyl)-4-phenyloxolane-2-carboxamide;
(2R,4R)-2-(2-methoxy-5-methylphenyl)-N-(2-methylquinoline-5-sulfonyl)-4-phenyloxolane-2-carboxamide;
(2S)—N-(2-aminoquinoline-5-sulfonyl)-2-(2-methoxy-5-methylphenyl)-4,4-dimethyloxolane-2-carboxamide;
(2R)—N-(2-aminoquinoline-5-sulfonyl)-2-(2-methoxy-5-methylphenyl)-4,4-dimethyloxolane-2-carboxamide;
(2S,4S)-2-(2-methoxy-5-methylphenyl)-N-(2-methylquinoline-5-sulfonyl)-4-phenyloxolane-2-carboxamide;
(2S,4R)-2-(2-methoxy-5-methylphenyl)-N-(2-methylquinoline-5-sulfonyl)-4-phenyloxolane-2-carboxamide;
(2R,4S)-2-(2-methoxy-5-methylphenyl)-4-(2-methoxyphenyl)-N-(2-methylquinoline-5-sulfonyl)oxolane-2-carboxamide;
(2R,4R)-2-(2-methoxy-5-methylphenyl)-4-(2-methoxyphenyl)-N-(2-methylquinoline-5-sulfonyl)oxolane-2-carboxamide;
(2R,4R)—N-(2-aminoquinoline-5-sulfonyl)-2-(2-methoxy-5-methylphenyl)-4-(2-methoxyphenyl)oxolane-2-carboxamide;
(2R,4R)—N-(2-aminoquinoline-5-sulfonyl)-2-(2-methoxy-5-methylphenyl)-4-phenyloxolane-2-carboxamide;
(2R,4R)-2-(2-methoxy-5-methylphenyl)-4-(3-methoxyphenyl)-N-(2-methylquinoline-5-sulfonyl)oxolane-2-carboxamide;
(2R,4S)-2-(2-methoxy-5-methylphenyl)-4-(3-methoxyphenyl)-N-(2-methylquinoline-5-sulfonyl)oxolane-2-carboxamide;
(2R,4R)-2-(2-methoxy-5-methylphenyl)-4-(4-methoxyphenyl)-N-(2-methylquinoline-5-sulfonyl)oxolane-2-carboxamide; and
(2R,4S)-2-(2-methoxy-5-methylphenyl)-4-(4-methoxyphenyl)-N-(2-methylquinoline-5-sulfonyl)oxolane-2-carboxamide.

16. The compound of claim 15, or a pharmaceutically acceptable salt thereof which is (2R,4R)-2-(2-methoxy-5-methylphenyl)-N-(2-methylquinoline-5-sulfonyl)-4-phenyloxolane-2-carboxamide.

17. The compound of claim 16 which is a pharmaceutically acceptable salt of (2R,4R)-2-(2-methoxy-5-methylphenyl)-N-(2-methylquinoline-5-sulfonyl)-4-phenyloxolane-2-carboxamide.

18. The compound of claim 16 which is (2R,4R)-2-(2-methoxy-5-methylphenyl)-N-(2-methylquinoline-5-sulfonyl)-4-phenyloxolane-2-carboxamide.

19. A pharmaceutical composition comprising a therapeutically effective amount of a compound of Formula (I) according to claim 1, or a pharmaceutically acceptable salt thereof, in combination with a pharmaceutically acceptable carrier.

20. A pharmaceutical composition comprising a compound of claim 1, or a pharmaceutically acceptable salt thereof, one or more potentiator, and one or more additional correctors.

21. A method for treating cystic fibrosis in a subject comprising administering a therapeutically effective amount of a compound of claim 1, or a pharmaceutically acceptable salt thereof, to a subject in need thereof.

22. The method of claim 21, further comprising administering a therapeutically effective amount of one or more potentiator; and administering a therapeutically effective amount of one or more additional correctors.
